SEQUENCE LISTING (30 sequences)

SEQ ID NO: 1 - RNA, 19 nt, synthetic construct
  source 1..19: mol_type = other RNA
cgtgcgggcccgggcgtgc

SEQ ID NO: 2 - RNA, 19 nt, synthetic construct
  source 1..19: mol_type = other RNA
cgtgccgctaccgctgcca

SEQ ID NO: 3 - RNA, 19 nt, synthetic construct
  source 1..19: mol_type = other RNA
cgtgctgacgccaccagac

SEQ ID NO: 4 - RNA, 19 nt, synthetic construct
  source 1..19: mol_type = other RNA
cgtgcccatcagccctggg

SEQ ID NO: 5 - RNA, 19 nt, synthetic construct
  source 1..19: mol_type = other RNA
ccgtgcccatcaccgtgcc

SEQ ID NO: 6 - RNA, 19 nt, synthetic construct
  source 1..19: mol_type = other RNA
cgtgcagccgagcgaggcc

SEQ ID NO: 7 - RNA, 19 nt, synthetic construct
  source 1..19: mol_type = other RNA
gcgtgcggccagtgaggac

SEQ ID NO: 8 - RNA, 19 nt, synthetic construct
  source 1..19: mol_type = other RNA
cgtgcaggccccgcccccg

SEQ ID NO: 9 - RNA, 19 nt, synthetic construct
  source 1..19: mol_type = other RNA
cgtgcagcccctgctctca

SEQ ID NO: 10 - RNA, 19 nt, synthetic construct
  source 1..19: mol_type = other RNA
cgtgcttggggagctagag

SEQ ID NO: 11 - RNA, 23 nt, synthetic construct
  source 1..23: mol_type = other RNA
gggcacggtggttcccatcaggg

SEQ ID NO: 12 - RNA, 23 nt, synthetic construct
  source 1..23: mol_type = other RNA
agaggggtccaggctgggcacgg

SEQ ID NO: 13 - RNA, 23 nt, synthetic construct
  source 1..23: mol_type = other RNA
ctcaacagaagaccaccaaatgg

SEQ ID NO: 14 - RNA, 23 nt, synthetic construct
  source 1..23: mol_type = other RNA
tctcccaccattgtctaggctgg

SEQ ID NO: 15 - RNA, 22 nt, synthetic construct
  source 1..22: mol_type = other RNA
cactctaaagggaggcactttg

SEQ ID NO: 16 - RNA, 25 nt, synthetic construct
  source 1..25: mol_type = other RNA
aacagaaagcgcttccctttggagg

SEQ ID NO: 17 - RNA, 22 nt, synthetic construct
  source 1..22: mol_type = other RNA
gcctctaaaaggaagcactttc

SEQ ID NO: 18 - RNA, 24 nt, synthetic construct
  source 1..24: mol_type = other RNA
acagaaagtgcttccctcaagagg

SEQ ID NO: 19 - RNA, 19 nt, synthetic construct
  source 1..19: mol_type = other RNA
cgtgcgggcccgggcgtgc

SEQ ID NO: 20 - RNA, 19 nt, synthetic construct
  source 1..19: mol_type = other RNA
cgtgcggggggccccgtgc

SEQ ID NO: 21 - RNA, 20 nt, synthetic construct
  source 1..20: mol_type = other RNA
gggcacggtggttcccatca

SEQ ID NO: 22 - RNA, 20 nt, synthetic construct
  source 1..20: mol_type = other RNA
agaggggtccaggctgggca

SEQ ID NO: 23 - RNA, 26 nt, synthetic construct
  source 1..26: mol_type = other RNA
taaaatggactatcatatgcttaccg

SEQ ID NO: 24 - RNA, 25 nt, synthetic construct
  source 1..25: mol_type = other RNA
aacaaaaaagcaccgactcggtgcc

SEQ ID NO: 25 - RNA, 24 nt, synthetic construct
  source 1..24: mol_type = other RNA
tttcgcccgctgcctggaagtatc

SEQ ID NO: 26 - RNA, 24 nt, synthetic construct
  source 1..24: mol_type = other RNA
ccgattctcccacctcagcctccc

SEQ ID NO: 27 - RNA, 24 nt, synthetic construct
  source 1..24: mol_type = other RNA
ttctggcggttctcttcagccggg

SEQ ID NO: 28 - RNA, 23 nt, synthetic construct
  source 1..23: mol_type = other RNA
cgggacggcccttctcctccggg

SEQ ID NO: 29 - RNA, 23 nt, synthetic construct
  source 1..23: mol_type = other RNA
gggcgcgacccacgctctgggcc

SEQ ID NO: 30 - RNA, 24 nt, synthetic construct
  source 1..24: mol_type = other RNA
ggcactagcgttgacgcagagccc

(19) United States
(12) Patent Application Publication
(10) Pub. No.: US 2026/0358329 A1
(43) Pub. Date: Nov. 12, 2026

(54) COMPOSITIONS AND METHODS TO DETECT AND INHIBIT NUCLEAR INVASION OF CYTOPLASM OR MITOCHONDRIA

(71) Applicant: H. LEE MOFFITT CANCER CENTER AND RESEARCH INSTITUTE INC., Tampa, FL (US)

(72) Inventors: Goodwin Jinesh Gerald Solomon Peter, Tampa, FL (US); Andrew Brohl, Tampa, FL (US)

(21) Appl. No.: 19/123,795

(22) PCT Filed: Nov. 30, 2023
(86) PCT No.: PCT/US2023/081746

§ 371 Date: Apr. 24, 2025

Related U.S. Application Data
(60) Provisional application No. 63/478,699, filed on Jan. 6, 2023.
(60) Provisional application No. 63/583,417, filed on Sep. 18, 2023.

(51) Int. Cl. G01N 33/50; C12N 15/113; C12Q 1/48; G01N 33/58

(52) U.S. Cl. CPC G01N 33/502; C12N 15/113; C12Q 1/48; G01N 33/582; C12N 2310/141; G01N 2333/912

(57) ABSTRACT

As disclosed herein, the cytoplasmic mitochondria and proteins can invade into the nucleus of cancer cells in the context of constitutive viral response cluster activation or overexpression. While this can be very dynamic process with a rapid invasion versus retraction cycles resulting in relatively complete nuclear and cytoplasmic compartmentalization, compositions and methods are provided to detect nuclear invasion of cytoplasm (NiC) and cytoplasmic mitochondria (NiCM) by inhibiting kinases that regulate influx and efflux of cargoes in and out of NiC. An RNA probe was developed which labels mitochondria when conjugated to a fluorochrome and is a part of NiCM detection.

### CROSS-REFERENCE TO RELATED APPLICATIONS

This application claims benefit of U.S. Provisional Application No. 63/478,699, filed Jan. 6, 2023, and U.S. Provisional Application No. 63/583,417, filed Sep. 18, 2023, which are hereby incorporated herein by reference in their entireties.

### STATEMENT REGARDING FEDERALLY SPONSORED RESEARCH OR DEVELOPMENT

This invention was made with Government Support under Grant No. CA255933 awarded by the National Institutes of Health. The Government has certain rights in the invention.

### SEQUENCE LISTING

This application contains a sequence listing filed in ST.26 format entitled “320803_2940_Sequence_Listing” created on Nov. 21, 2023, and having 27,520 bytes. The content of the sequence listing is incorporated herein in its entirety.

### BACKGROUND

It is a universal fact that cells organize themselves into nucleus and cytoplasm as two prominent compartments to facilitate transcription primarily within nucleus and protein translation primarily within cytoplasm or inside mitochondria. Under certain physiologic conditions such as mitosis, the nuclear membrane disappear by a process called “nuclear envelope breakdown (NEBD)” to result in the mixing of both cytoplasm and nuclear content to facilitate cell division. However, upon completion of daughter cell segregation the nucleus and cytoplasm quickly reform by a process called “nuclear envelope reassembly” to separate cytoplasm from nucleus. To date, it is not known that the cytoplasm or its organelles such as mitochondria can invade into the nucleus and if it does, the functional context is not known.

### SUMMARY

As disclosed herein, the cytoplasmic mitochondria can invade into the nucleus of cancer cells in the context of constitutive chromosome-19 viral response micro-RNA (miRNA: C19MC) cluster activation or overexpression. While this can be very dynamic process with a rapid invasion versus retraction cycles resulting in relatively complete nuclear and cytoplasmic compartmentalization, a method was identified to detect the nuclear invasion of cytoplasm (NiC) and cytoplasmic mitochondria (NiCM).

Disclosed herein is a method for detecting nuclear invasion of cytoplasm (NiC) in cells, that involves contacting the cell with an effective amount of Aurora-B kinase inhibitor (e.g. 20 nM for 24 hours), and staining the nuclear DNA of the cell with a live nuclear dye (e.g. Hoechst-33342), wherein detection of unstained regions within the nucleus of the cell is an indication of NiC.

In some embodiments, the Aurora-B kinase inhibitor is AZD-1152-HQPA, ZM 447439, PF-03814735, MK-0457, CCT137690, AMG 900, MLN8237, SNS-314 (mesylate), GSK1070916, Danusertib, CYC-116, CCT129202, or PHA-680632.

In some embodiments, the nuclear dye is Hoechst-33342 for live cells. In some embodiments, the nuclear dye is DAPI, propidium iodide or any other nuclear DNA stain can be used in fixed and permeabilized cells.

In some embodiments, the method further involves contacting the cell with an effective amount (e.g. 1 nM) of human chorionic gonadotropin (hCG: A and B polypeptide combination a ligand that promotes steroidogenesis), an Aurora-B inhibitor, or a combination thereof.

For example, in some embodiments, all-trans-retinoic acid (ATRA, e.g. 1 μM), arachidonic acid, diacylglycerol, or Phorbol 12-myristate 13-acetate (PMA) can be used with hCG and/or Aurora-B inhibitor to promote nuclear invasion of cytoplasm.

In some embodiments, the cytoplasmic mitochondria invasion into nucleus is validated by contact the cell with a mitochondrial dye or probe after performing the afore mentioned methods, wherein detection of mitochondria within the DNA unstained regions within the nucleus is an indication of nuclear invasion of cytoplasmic mitochondria (NiCM). For example, in some embodiments, the mitochondrial dye is JC1, Rhodamine-123, mitotracker dye, or genetic agents such as fluorescent protein (GFP/RFP/CFP/tdTomato/mCherry and so on)-tagged mitochondria localization signals.

In some embodiments, the mitochondrial probe is an RNA probe having a detectable label and a sequence that partially overlaps with CIC (Capicua Transcriptional Repressor) RNA, that can be used to detect NiC. For example, in some embodiments, the mitochondrial RNA probe has the nucleic acid sequence

(SEQ ID NO: 1)
CGUGCGGGCCCGGGCGUGC,
(SEQ ID NO: 2)
CGUGCCGCUACCGCUGCCA,
(SEQ ID NO: 3)
CGUGCUGACGCCACCAGAC,
(SEQ ID NO: 4)
CGUGCCCAUCAGCCCUGGG,
(SEQ ID NO: 5)
CCGUGCCCAUCACCGUGCC,
(SEQ ID NO: 6)
CGUGCAGCCGAGCGAGGCC,
(SEQ ID NO: 7)
GCGUGCGGCCAGUGAGGAC,
(SEQ ID NO: 8)
CGUGCAGGCCCCGCCCCCG,
(SEQ ID NO: 9)
CGUGCAGCCCCUGCUCUCA,
or
(SEQ ID NO: 10)
CGUGCUUGGGGAGCUAGAG.

In some embodiments, the detectable label is a fluorochrome. In some embodiments, the cyanine dye, such as Cy5.

In some embodiments, the disclosed methods can be used with any cell where NiC is possible. For example, in some embodiments, the cell is a cancer cell.

Also disclosed method for detecting mitochondria within a cell that involves transfecting the cell with an effective amount of the RNA probe (e.g. 10 nM) disclosed herein, and imaging the cell for the detectable label after treating with Aurora-B inhibitor or its aforementioned combinations.

In some embodiments, the disclosed methods can be used to inhibit NiC.

In some embodiments, the disclosed methods can also be used to visualize cytoplasmic compartment of cells (mitochondria) without NiC especially when Aurora-B inhibitor is not used. In some embodiments, the disclosed methods can also be used to detect nuclear division. In some embodiments, the disclosed methods can also be used to track cells that generate genomic instability.

In some embodiments, the RNA probe can be used for live cell imaging using time lapse microscopy. In some embodiments, the RNA probe can be used to track mitochondria and mitochondrial dynamics such as mitophagy, mitochondrial fission and mitochondrial fusion.

In some embodiments, the RNA probe is attached to a nuclear transcribed RNA of interest in order to import the RNA of interest into mitochondria. For example, in some embodiments, the RNA of interest is used to induce mitochondrial apoptosis, e.g. for cancer therapy.

In some embodiments, the RNA probe is used to study trophoblast giant cell formation in vivo or their differentiation in vitro. For example, in fertility, miscarriage or eclampsia studies. In some embodiments, the RNA probe is used to study non-sense mediated RNA decay in vitro.

In some embodiments, the RNA probe in combination with Aurora-B inhibitor can be used to detect virus infected cells or cells with constitutive activation of antiviral C19MC miRNA cluster.

Also disclosed herein is a set of RNA probes comprising the nucleic acid sequence GGGCACGGTGGTTCCCATCAGGG (SEQ ID NO:11) and AGAGGGGTCCAGGCTGGGCACGG (SEQ ID NO:12). These are referred to herein as CRISPR-Chopper-guide gRNAs-1&2 (Chopper 1 and Chopper 2), respectively. These can be used to delete 13-repeats within C19MC upstream CpG island (CpG86). As a result, they can be used to restore anti-viral C19MC miRNA expression in certain cell systems, study NiC in cell systems, override SpCas9-mediated suppression of antiviral-C19MC, and in antiviral and cancer therapeutic contexts. Choppers-1&2 gRNAs have far-reaching applications to study, such as eclampsia, fertility, antiviral research, cancer metastasis, and immunotherapy.

Also disclosed herein is a set of RNA probes comprising the nucleic acid sequence CTCAACAGAAGACCACCAAATGG (SEQ ID NO: 13) and TCTCCCACCATTGTCTAGGCTGG (SEQ ID NO:14). These are referred to herein as ZNF331-C19MC Fusion. They can also regulate C19MC miRNAs but by a different mechanism than the Chopper gRNAs.

Therefore, also disclosed is a method for restoring anti-viral C19MC miRNA expression in a cell, comprising contact the cell with the set of guide RNA probes of claim 26 under conditions suitable for gene editing.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1L show C19MC miRNAs promote embryonic stemness and interfere with meiotic nuclear division through meiotic bridged chromosomes. FIG. 1A shows cumulative C19MC expression across various cancer types in TCGA dataset. Percentage overexpression beyond pan-normal cut-off (dotted line) is indicated on right. Cancer types in FIG. 1A were further subjected to C19MCHigh/Low GSEA differential gene expression in FIGS. 1B, 1F and 1J where a meiotic (excluding mitotic), ESC, DNA-damage response (DDR) enrichments were grouped respectively. FIG. 1C shows rare meiotic figures in MCF-7 cells where chromosomes condensed towards nuclear membrane. FIG. 1D shows a novel type of meiosis-III with nuclear cleavage furrow (NCF) and cytokinesis cleavage furrow (CCF) with ESCRT ring at different planes. FIG. 1E shows sickle nuclei count in C19MC miRNA overexpressed MCF-7 cells. Note the aberrant DNA accumulation at NCFs. FIGS. 1G and 1H show embryonic nuclear division visualized with Cy5-CSi000A probe. * vacuole. FIG. 1I shows C19MC-induced poor differentiation status is reversible by ATRA. FIG. 1K shows meiotic bridged chromosomes (MBCs) efficiently generated by miR-519D (FIG. 6L-6M). FIG. 1L shows anti-miR-519D reverses sickle nuclear phenotype in MCF-7-519D stable cells to show specificity.

FIGS. 2A to 2M show nucle(ol)ar invasion of cytoplasmic-materials (RPS6, nucleolin, REST, and mitochondria) leads to meiotic nuclear division (MND). FIG. 2A shows baseline sickle nuclei (ref arrow) and MND percentages in C19MCHigh cell line WM793 (NCF, blue arrows; DNA, Hoechst-33342 live-stained). FIG. 2B shows GSEA enrichments in 20 TCGA C19MCHigh cancer types showing Aurora-B-related gene-signatures. FIG. 2C shows Aurora-B inhibition results in nucle (ol) ar invasion of cytoplasmic-materials (NiC, green arrow). FIG. 2D shows emerin-labelled nuclear membrane showing NiC process (panel-A) to form a nuclear membrane bound NIC compartment (NM-NiC, panels-B) and emerin-strings (eStrings) that hold the sickle nuclear shape upon opening-up of the NiC compartment (panel-C). FIGS. 2E and 2F show localization of mitochondria using JC-1 staining (FIG. 2E) and using Cy5-CSi000A RNA probe (FIG. 2F) within NiC. A nucle (ol) ar invasive cytoplasmic bridge (NiCB) is indicated in panel-e. Please see FIGS. 8G and 9B for schematic details on NiCB. FIGS. 2G and 2H show meiotic promoter hCG promotes the size of NiC and multinucleation in WM793 cells. FIG. 2I shows meiotic promoter ATRA enhances the size of NiC promoted by hCG in WM793 cells. FIG. 2J shows HIV+IAV-signature inhibitors and CDK1 inhibitors impede NiC formation (see FIG. 7N to see why CDK1 inhibitor was used). FIG. 2K shows inhibition of C19MC miRs with anti-miR-519D impedes NiC and MND frequency. FIGS. 2L and 2M show immunofluorescence and ImageJ quantification of protein cargo traffic (RPS6, nucleolin, and REST/NRSF) into the NiC compartment (anti-C19MC-miRs used to check the C19MC dependency).

FIGS. 3A to 3H show CpG86-regulated C19MC miRNAs are required for NiC formation and, protein and mitochondria cargo traffic into NiC. FIG. 3A shows CRISPR strategy to knock-out repeats within C19MC upstream-CpG86. Arrows indicate the screening primer locations and directionality (F/R, forward/reverse). Bottom left inset: Chopper pooled WM793 cells (Chop. P) showing the CRISPR knock out of CpG86 repeats. Bottom right inset: qRT-PCR evaluation of C19MC miRNAs in CRISPR engineered cells. NGP: SpCas9 control pooled WM793 cells. FIG. 3B shows evaluation of SpCas9 at genomic, transcription and protein levels by PCR, RT-PCR and Western immunoblotting. FIG. 3C shows CRISPR-choppers-1 and 2 degrade SpCas9 through proteasomes as evaluated by Western immunoblotting with MG-132. FIG. 3D shows immunofluorescence quantification showing the suppression of SYCP2 levels by SpCas9 and its restoration by choppers. FIG. 3E shows SpCas9 suppresses NiC formation and CRISPR-choppers restores it, horizontal dotted line is a gate to calculate the percentage of large NiC positive cells. FIG. 3F to 3H shows immunofluorescence quantification of protein cargos nucleolin (FIG. 3F), RPS6 (FIG. 3G) and Cy5-CSi000A staining and quantification of organelle cargo mitochondria (panel-h) in CRISPR engineered cells to show the C19MC dependence of cargo traffic into the NiC compartment as evaluated under Aurora-B inhibited conditions. Vertical green dotted lines are gates used to calculate the percentage difference in traffic between cell types. nDNA, nuclear DNA; AU, arbitrary units scaled down; n=300 data points from replicates.

FIGS. 4A to 4I shows C19MC miRNAs and autophagy direct nuclear coverage of mitochondria and meiotic nuclear cleavage furrow ingression. FIG. 4A shows starvation-induced autophagy doubles the NiC positivity while impairing DNA synthesis in WM793 cells. AU, scaled down arbitrary units. Grey dotted line: G1 stage, and black dotted dine G2 stage of cell cycle. FIG. 4B shows autophagy inhibition augments the NiC size under fed conditions. Only NiC forming cells were measured. dotted line: discriminatory gate for percentage calculations. FIG. 4C shows autophagic flux within sub-cellular locations under Aurora-B inhibited condition: NiC is an autophagy immune zone as GFP-LC3 accumulate at NiC but degraded at cytoplasm. See FIG. 9A for cytoplasmic/perinuclear GFP-LC3. FIG. 4D shows autophagy drives NCF ingression from cytoplasmic side. Total MND is shown as bars and sub-types as data points. NiC is without any NCF ingression, NCFI is with NCF ingression, NC is with nuclear cleavage that opened up at least at one point, NiC+MND is dual NiC with NCF. FIG. 4E shows NiC inhibition promotes DNA synthesis to a triploid content (dotted line). FIG. 4F shows baseline DNA intensity levels between CRISPR engineered and not engineered cell types. Black dotted line, diploid level. FIG. 4G shows Cy5-CSI000A labeling and quantification of mitochondrial load/cell in CpG86-CRISPR-engineered cells. FIGS. 4H to 4I show baseline and NiC altering drug induced changes in nuclear coverage of mitochondria (NCOM: violin plots). Relative mitochondrial load/cell were displayed in aligned dot plots. CQ, chloroquine. See FIG. 9B for a schematic depicting how NCOM helps passive mitochondria and potentially mitochondrial ribosome traffic into the NiC compartment along with invading nuclear membrane.

FIG. 5 shows a C19MC-directed meiotic nuclear division pathway translated and validated based on TCGA C19MCHigh pan-cancer transcriptome. A rDNA ribosome biogenesis pathway that includes IAV+HIV infection gene signatures holds the control over nucle (ol) ar invasion of cytoplasmic (NiC) materials (cargos). Bacterial SpCas9 suppress this effect while CpG86-repeat deletion unleashes C19MC miRNAs to super-regulate the NiC. Autophagy further divides the NiC positive nuclei to generate daughter nuclei by promoting NCF from cytoplasmic side. Aurora-B functional status further determines the cytokinesis to generate daughter cells but is hampered by meiotic bridged chromosome to generate meiotic-bridged mini-nuclei (syncytium). Daughter cells engulf other daughter cells to bring target cell-nuclear DNA into cytoplasmic vacuoles where mitochondria can fuse with nDNA to generate embryonic nuclear division which can be visualized with CSi000A but not with Hoechst-33342. DDR, DNA-damage repair pathway; NiCB, Nucle (ol) ar-invasive cytoplasmic bridge; NCOM, Nuclear coverage of mitochondria. The ribosomes depicted are mitochondrial ribosomes as RPS6 could be from individual proteins or from ribosomes of various cyto-locations.

FIGS. 6A to 6N show C19MC, embryonic stemness, and chromosomal instability in the context of meiosis in cancer cells. FIG. 6A shows EnrichR analysis and R visualization of meiosis signature enriched in C19MC^{High} cancers (MSigDB) to find matching cell lines in NCI 60 cell lines panel. FIG. 6B shows TCGA histology slide of C19MC^{High} human breast cancer (BRCA-TCGA-D8-A1X5-01A) showing meiosis-III in vivo. The black boundary lines mark the “to-be” daughter cells, black arrows indicate the cytokinetic cleavage furrow (CCF), red arrows indicate the nuclear cleavage furrows (NCF). DNA, hematoxylin-stained DNA in purple. FIG. 6C shows meiosis-III daughter cells imaged after nocodazole arrest and isolation of meiosis-III cells from MCF-7 cells and release for 2 hours in complete MEM for ESCRT-cut progression to generate daughter cells (green arrow). NCF, nuclear cleavage furrow; DNA, Hoechst-33342 stained DNA. Note the condensation differences in sickle nucleus and the other nucleus of a single daughter cell. FIG. 6D shows GSEA enrichments in C19MC^{High/Low} tumors of 20 different cancer types showing metal response-related gene-signatures. FIG. 6E shows quantitative real-time PCR of C19MC miRNA upregulation in zinc resistant versus TPEN (zinc chelator) resistant MCF-7 cells generated for 20 days (left panel). Promotion of sickle nuclei under the zinc resistant condition versus parental control is shown in right panel. FIG. 6F shows immunofluorescence and quantification of synaptonemal complex protein-2 (SYCP2) positive cell percentage in C19MC miR stably overexpressed MCF-7 cells versus control pMir cells. Five fields from replicates were quantified. FIG. 6G shows live staining of mitochondria using 30 nM Cy5-labelled CSi000A RNA probe. Cells were stained with CSi000A for 24 hours and counter live-stained with Hoechst-33342 to visualize nuclear DNA. FIG. 6H shows sickle nucleated MCF-7-miR-519D stably transfected cell encompassing target cell for entosis/phagocytosis. Mitochondria and nuclear DNAs were live stained with Cy5-CSi000A and Hoechst-33342 respectively. FIG. 6I shows embryonic nuclear division (END) in MCF-7-miR-519D stably transfected live cells that usually happens within vacuoles (marked by an asterisk) after entosis/phagocytosis. Mitochondria and nuclear DNAs were live stained with Cy5-CSi000A and Hoechst-33342 respectively. Purple arrow, sickle nucleus; Blue arrows, nuclear cleavage furrow (NCF); Yellow arrows, embryonic nuclear cleavage furrow (eNCF); BF, bright field. FIG. 6J shows entosis (Top three panels) and embryonic nuclear division (END: bottom two panels) in vivo in human breast cancer (BRCA-TCGA-D8-A1X5-01A). Sickle nucleated breast cancer cells (Black arrows) engulfed target cells (Red arrows) by entosis/phagocytosis as it is localized within vacuoles (black asterisks in top three panels). Engulfed targets cells undergo embryonic nuclear division (eNCF: Yellow arrows) within vacuoles (Green asterisks in bottom two panels). FIG. 6K shows colony morphology count of C19MC miRNA stably transfected MCF-7 cells versus control pMir-transfected cells. FIG. 6L shows structural genomic instability in C19MC stably transfected MCF-7 cells. Note the chromosome accumulation (Yellow arrow heads in zoom panel) at the nuclear cleavage furrow (NCF: blue arrows) during meiotic nuclear division (MND) in left panel set. Note the meiotic bridged chromosomes (MBCs: arrow heads in zoom panel) with T-type bridges (Yellow arrow heads) and X-type bridges (Green arrow heads) are at high frequencies in 519D transfected cells (Middle panel set). Anaphasic bridges at cytokinesis stage (Purple arrow heads in zoom panel) as a result of separase defects (Centromeric cohesion retained) were frequently detected in C19MC miRNA transfected cells than in control pMir transfected cells (has no bridges, Black arrow heads) in right panel set. FIG. 6M shows structural genomic instability: Meiotic bridged chromosomes result in the formation of meiotic-bridged-mini-nuclei upon ESCRT failure to cut daughter cells off. Hypotonic isolated nuclei were Giemsa stained and imaged to show the bridges between nuclei and mini-nuclei in a bead-string appearance (Top four panels: Blue arrows, NCF; Yellow arrows, mini-nuclei; Black arrows, swollen sickle nuclei). Bottom panel: Cytogenetic analysis showing chromosome sequestering of meiotic bridged chromosomes (MBC) from *non-bridged (mitotic condensed) chromosomes (NBC) separately into daughter nuclei. FIG. 6N shows numerical genomic instability analysis by cytogenetics and Giemsa staining in C19MC miRNA stably transfected MCF-7 cells. n=25*countable chromosome spreads each. Note: a large proportion of cells were not countable due to meiotic bridged chromosomes.

FIGS. 7A to 7P show a cluster-wide C19MC hyper-expressing system reveals the regulation of NiC by hCG and rDNA gene-signatures. FIG. 7A shows GSEA enrichments in C19MC^{High/Low} tumors of 20 different cancer types showing various virus-related gene-signatures. FIG. 7B shows influenza-A virus (IAV) and HIV infection signature genes (MSigDB) sorted for specific and shared genes and examined for expression in CCLE cell line database 1020 cell lines. FIG. 7C shows EnrichR analysis and R visualization of influenza signature (MSigDB) enriched 100 genes in C19MC^{High} TGCTs to find matching cell lines in CCLE proteomics cell line panel. A few gene signatures were explicitly indicated for the virus name in this figure, for viral protein/gene involved in the original study referenced in MSigDB. FIG. 7D shows schematic of C19MC genomic location (Left panel) and its cluster-wide expression in WM793 cells in CCLE miRNASeq data (Right panel). The cumulative expression of all C19MC miRNAs in CCLE cell lines is shown in FIG. 7E. FIG. 7F shows quantitative real-time PCR evaluation of C19MC miRNA hyper-expression in WM793 cells (RNU6B normalized), in comparison to A375 and IPC298 cell lines. FIG. 7G shows meiotic nuclear division in WM793 cells. Green arrow heads, Nuclear-membrane anchored chromosomes (NAC); Blue arrows, Nuclear cleavage furrow (NCF); Yellow arrow head, Meiotic bridged chromosomes (MBCs). FIG. 7H shows tracking GSEA enrichments in C19MC^{High/Low} tumors of 20 different cancer types for ESCRT and anaphase-promoting complex (APC)-related gene-signatures (Top dot plot). A schematic showing the directionality of APC to ESCRT function in midbody abscission depending on Aurora-B functional status is shown in bottom panel. FIG. 7I shows single or dual nature of nucle (ol) ar invasive cytoplasm (NiC, green arrows) in WM793 cells inhibited for Aurora-B for 24 hours and live stained with Hoechst-33342. FIG. 7J shows arresting WM793 meiotic cells with aurora-B inhibitor after isolating cells using 16-hour nocodazole treatment-coupled mitotic shake-off to show NiC (Green arrows), and nuclear cleavage furrows (Blue arrows). FIG. 7K shows hypotonic nuclear isolation and Giemsa staining of Aurora-B inhibited MCF-7-519D stable cells to show that, NiC is a closed compartment with Giemsa positive DNA. Hoechst-33342 and CSi000A were stained after Giemsa staining to visualize and confirm NiC location and Hoechst-free DNA is packed in NiC. FIG. 7L shows immunofluorescence localization of RPS6, nucleolin and REST/NRSF within NiC compartment in Aurora-B inhibited WM793 cells. FIG. 7M shows bird's eye view volcano plot analysis of gene enrichments to show that CGA, and CGB (components of the pregnancy hormone and meiotic promoter human chorionic gonadotrophin, hCG: inset schematic) as candidate genes promoted in C19MC^{High} TCGA and THYM, two top C19MC expressing cancer types. FIG. 7N shows influenza-A virus (IAV) and HIV infection signature genes (red font) regulate the transcription, translation, nuclear import & export, and quality control (proteasomes) together constitutes the rDNA expression pathway and inhibitors of various arms (Orange-red gradient boxes) of these signatures are depicted. CDK1 (green font), a promoter of both rDNA transcription and rRNA translation and its inhibitor Ro3306 is also shown. FIGS. 7O to 7P shows vehicle control DMSO (used in most inhibitor experiments) or Cy5-CSi000A RNA does not affect NiC or MND patterns as indicated by non-significant (NS) p-values.

FIGS. 8A to 8E show CpG86 regulates C19MC, SpCas9 proteasomal degradation and cargo traffic into the NiC compartment. FIG. 8A shows CpG86 13-repeat region is a hypervariability region. 19 cell line genomic DNAs were amplified for 13-repeat regions (see FIG. 3A for primer locations) to evaluate variability in amplicon size. FIG. 8B shows GSEA enrichments of ubiquitin and proteasome-related gene signatures in C19MC^{High/Low} tumors of 20 different cancer types. FIG. 8C shows immunofluorescence localization and quantification of SpCas9 protein in WM793 NGP cells and in Chopper cells after incubation of cells with proteasome inhibitor for 24-hours. Note the predominant localization of SpCas9 in cytoplasm (in WM793-NGP cells), perinuclear region (in WM793-chopper cells) and rarely inside NIC (in WM793-chopper cells) on the left side panels. The right-side panel shows the quantification in terms of percentage of cells where the NGP and chopper cells are identified by black and red data points respectively. Rare nuclear localization is also noted. FIG. 8D shows immunofluorescence localization of β-Tubulin in Aurora-B inhibited WM793 cells showing NiC localization of β-Tubulin (in few clusters of cells while majority of the cells lack β-Tubulin). FIG. 8E shows protein (left) and organelle (right) traffic models of NiC formation. Protein cargos can use the nuclear import and export route to reach NiC despite there is a possibility that the nuclear invasive cytoplasmic bridge (NiCB) could aid this traffic. Organelle traffic through nuclear pore complex (NPCs) could be limited by size as no known report of organelle traffic through NPCs except for ribosomal subunits. Therefore, a MTOC dependent perinuclear push of organelles such as mitochondria through NiCB is proposed.

FIGS. 9A to 9D show autophagy and nuclear coverage of mitochondria direct MND in vitro. FIG. 9A shows transient overexpression of GFP-LC3-RFP plasmid in WM793 cells to show that, RFP lost expression in living cells but not in dying cells (Black arrows) whereas live cells express GFP-LC3 in perinuclear cytoplasmic region. Selective loss of RFP in live cells indicates the cleavage of RFP from GFP-LC3 potentially by ATG4 (Kaizuka, T. et al. Mol Cell. 2016 64(4):835-849). FIG. 9B shows nuclear coverage of mitochondria (NCOM) can passively slide the mitochondria tethered to the outer nuclear membrane as part of NiC formation through nuclear invasive cytoplasmic bridge (NiCB). Mitochondrial ribosomes (mtRibosomes) can have a piggy-back ride into the NiC compartment or alternatively, the mitochondria are loaded with more ribosomes at NiC as a pit-stop for re-fuel. MT, microtubule; Abi, Aurora-B inhibitor. The nuclear membrane traffic directionality is indicated by small black arrows in bottom panel. FIG. 9C show live mitochondria and nuclear DNA staining of MCF-7 cells using Cy5-CSi000A (24-hour) and Hoechst-33342 (20 minutes before imaging) in Aurora-B inhibited (24-hour) condition showing nuclear membrane margination of nuclear DNA and mitochondria in cells undergoing meiotic nuclear division (MND). FIG. 9D shows multipolar MND in Aurora-B inhibited WM793 cells showing RPS6 at NiC re-establish connections with main cytoplasm (immunofluorescence).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20 °C and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “detectable label” refers to any moiety that can be selectively detected in a screening assay. Examples include without limitation, radiolabels, (e.g., ³H, ¹⁴C, ³⁵S, ¹²⁵I, ¹³¹I), affinity tags (e.g. biotin/avidin or streptavidin, binding sites for antibodies, metal binding domains, epitope tags, FLASH binding domains, fluorescent or luminescent moieties (e.g. fluorescein and derivatives, GFP, rhodamine and derivatives, lanthanides etc.), and enzymatic moieties (e.g. horseradish peroxidase, β-galactosidase, β-lactamase, luciferase, alkaline phosphatase). Such detectable labels can be formed in situ, for example, through use of an unlabeled primary antibody which can be detected by a secondary antibody having an attached detectable label.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages).

Examples of fluorescent labels include, but are not limited to: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rhol I, ATTO Rhol2, ATTO Thiol 2, ATTO RholOI, ATTO 590, ATTO 594, ATTO Rhol3, ATTO 610, ATTO 620, ATTO Rhol4, ATTO 633, ATTO 647, ATTO 647N, ATTO 655, ATTO Oxal2, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, quantum dots, and a tethered fluorescent protein. Examples of AlexaFluor dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: C19MC Drives Nucleolar Invasion of Mitochondria and Meiotic Nuclear Division in Human Cancers

### Summary

Chromosome-19 miRNA cluster (C19MC) restricts viruses depending on the multinucleated-state of placental trophoblasts (Delorme-Axford, E. et al. Proc Natl Acad Sci USA. 2013 110(29):12048-53; Delorme-Axford, E., et al. Annu Rev Virol. 2014 1(1):133-46). However, the relationship of C19MC to multinucleation is unknown. Here we show that, C19MC is overexpressed in multiple cancer-subsets, with meiosis-related gene-signatures. We discover a novel meiosis-III with parallel progression of meiotic-nuclear division (MND) and cytokinesis where, C19MC-miRNAs promote meiotic-bridged-chromosomes (MBC) to delay the progression of MND and generate multinucleated cells. MND starts with the invagination of nuclear membrane to form nucle (ol) ar-invasive cytoplasmic compartment (NiC: includes cargos such as mitochondria, ribosomal protein-S6, REST, and nucleolin). Aurora-B regulates the efflux of cargos from NiC to prevent MND, whereas, influenza+HIV infection signatures (rDNA pathway: rDNA-transcription, rRNA-translation, nuclear export, proteasome), CDK1, and autophagy promote cargo influx into the NiC to promote MND when Aurora-B is inhibited. We further discovered a novel 13-repeat-genomic-switch at the C19MC-upstream-CpG86, which silences C19MC expression, and abolishes NiC-formation upon bacterial-SpCas9 introduction. CRISPR-deletion of this genomic-switch promotes proteasomal-degradation of SpCas9 to restore C19MC-miRNA expression and, NiC-formation upon Aurora-B inhibition. Therefore, C19MC-miRNAs promote multinucleation through MND, depending on Aurora-B functional-status, and is super-regulated by bacterial-SpCas9 at CpG86. This discovery has impacts in cancer-pathogen interactions and immunotherapy, vertical transmission of viruses in human/primate-placenta, antiviral-research and SpCas9-directed CRISPR-therapeutics.

Vertical transmission of viruses across placenta (the interface between fetus and mother) is restricted by the multinucleated placental syncytiotrophoblasts but not by the mononucleated cytotrophoblasts (Delorme-Axford, E., et al. Annu Rev Virol. 2014 1(1):133-46). C19MC is an antiviral response miRNA cluster with 46 highly similar but distinct miRNAs (Jinesh, G. G. & Brohl, A. S. Biol Rev Camb Philos Soc. 2020 95(1):244-266; Bortolin-Cavaillé, M. L., et al. Nucleic Acids Res. 2009 37(10):3464-73) which is expressed primarily in placenta and plasma of pregnant women (Bortolin-Cavaillé, M. L., et al. Nucleic Acids Res. 2009 37(10):3464-73). However, it is not known whether the cells acquire multinucleation status because of viral proteins or due to C19MC. In cancers, multinucleated as well as giant nucleated cells with chromosomal instability are considered tumorigenic stem cells (Jinesh, G. G. & Kamat, A. M. Cancer Res. 2017 77(22):6144-6156; Fujiwara, T. et al. Nature. 2005 437(7061):1043-7; Holland, A. J. & Cleveland, D. W. Nat Rev Mol Cell Biol. 2009 10(7):478-87). Interestingly, a few rare pediatric cancers such as undifferentiated embryonal sarcoma of the liver (UESL) (Setty, B. A. et al. PLOS Genet. 2020 16(4): e1008642) and embryonal tumors with multilayered rosettes of brain (ETMR) (Archer, T. C. & Pomeroy, S. L. Nat Genet. 2014 46(1):2-3) overexpress C19MC which is associated with chromosomal translocation-assisted fusion of C19MC with other genes such as ZIM2/PEG3 (Setty, B. A. et al. PLOS Genet. 2020 16(4): e1008642), TTYH1 (Archer, T. C. & Pomeroy, S. L. Nat Genet. 2014 46(1):2-3). However, the genetic switch that regulates C19MC expression remains elusive. While we previously investigated C19MC expression in triple negative breast cancer (Jinesh, G. G., et al. PLOS One. 2018 13(10): e0206008) and hepatocellular carcinoma (Jinesh, G. G. et al. Sci Rep. 2021 11(1):12673; Jinesh, G. G. et al. Sci Rep. 2020 10(1):12371), we comprehensively examined The Cancer Genome Atlas (TCGA) data in this study to get insights into the function and regulation of C19MC in cancers, and validate the signatures of C19MC^{High} cancers in vitro, which might in future translate to a novel regulatory pathway restricting viruses in cancer and vertical transmission of viruses in human/primate placenta.

### Methods

### Pan-Cancer Clinical Datasets and Data Processing

The results in this study are in part based upon the data generated by the TCGA (The Cancer Genome Atlas) Research Network. TCGA miRNA-seq, and RNA-seq, data for all available cancer types [Adrenocortical carcinoma (ACC), Bladder urothelial carcinoma (BLCA), Breast invasive carcinoma (BRCA), Cervical and endocervical cancers (CESC), Cholangiocarcinoma (CHOL), Colon adenocarcinoma (COAD), Rectum adenocarcinoma (READ), Diffuse Large B-cell Lymphoma (DLBC), Esophageal carcinoma (ESCA), Glioblastoma multiforme (GBM), Brain Lower Grade Glioma (LGG), Head and Neck squamous cell carcinoma (HNSC), Kidney Chromophobe (KICH), Kidney renal clear cell carcinoma (KIRC), Kidney renal papillary cell carcinoma (KIRP), Acute Myeloid Leukemia (LAML), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM)] were from the Broad Institute.

Setting up the C19MC^{High} and C19MC^{Low} groups and integration of miRNASeq and RNAseq data within each cancer types:

Based on the normal (matched, as per TCGA-IDs) samples in all cancer types (excluding a few cancer types which do not have normal samples) we set up a cumulative C19MC miRNA expression cut-off of 48.25 (reads per million) at 98 percentiles of pan-normal C19MC expression. Based on cumulative expression of all 46 C19MC miRNAs 13/33 cancer types (PCPG, MESO, COAD, GBM, KIRC, PRAD, READ, LGG, PAAD, LAML, KICH, ACC, and CHOL) were not subjected to further analyses due to no or low number of tumors with C19MC expression above the C19MC expression cut-off value. The remaining 20 cancer types were classified into C19MC^{High} and C19MC^{Low} groups with equal number of tumors/patients on each group based on the number of tumors available on both extremes of the C19MC expression spectrum.

Integration of miRNA-Seq and RNA-Seq Datasets of 20 Cancer Types:

Based on the C19MC^{High} and C19MC^{Low} group TCGA samples, the barcodes of miRNA-seq datasets were integrated to the RNA-seq datasets of the corresponding cancer type. The patients who do not have RNA-seq data matching to miRNA-seq data or vice versa were omitted and the group size were kept equal accordingly to have equal statistical power during downstream analyses.

### IBC Approval

Use of all CRISPR guides were approved by Institutional Biosafety Committee (IBC-University of South Florida: PROTO2022-041) and were used as plasmids instead of viruses.

C19MC^{High} Versus C19MC^{Low} Gene-Set Enrichment Analyses (GSEAs) and Visualizations in R

The data of C19MC^{High} and C19MC^{Low} groups of 20 cancer types that had C19MC expression values above the cut-off value were subjected to FPKM adjustment and group averages for all individual genes were calculated. The group averages were subjected to GSEA analyses for Hallmark, C1-positional, C2-curated, C3-motif, C5-Gene ontology, C6-Oncogenic and C7-Immunologic complete gene-set collections/modules for humans (MSigDB: version 7.2). The phenotypes analyzed were always C19MC^{High} versus C19MC^{Low} tumors and thus the normalized enrichment scores (NES) and FDRq values represent enrichments in C19MC^{High} tumors compared to corresponding C19MC^{Low} tumors. We generated 15060 enrichment results per cancer type and a total of 301,200 enrichments for 20 cancer types (excluding C1 positional set enrichments). All module enrichment results were combined for each cancer type to rank enrichments. Top ranked gene-sets (positive and negative NES) with lowest FDRq-value in C19MC^{High} tumors of each cancer type is tracked across all 20 cancer types irrespective of the enrichment status in remaining cancer types. Dot plots were generated in R using ggplot2 package (R version 4.1.3). NES and FDRq values categorized based on functional groups and enrichment across more cancer types for further downstream investigations. Currently, we excluded immune signatures except a few, as this study focuses on a cell biological phenomenon. FDRq values were converted to negative integers so as to reflect the dot size smaller for false discoveries. For all TCGA 20-cancer type GSEA enrichment dot plots, the NES and FDRq scales were kept constant (−3.5 to 3.5 for NES and 0 to 1 for FDRq), so that one can compare an enrichment with any other TCGA-20 cancer-dot plot in this study.

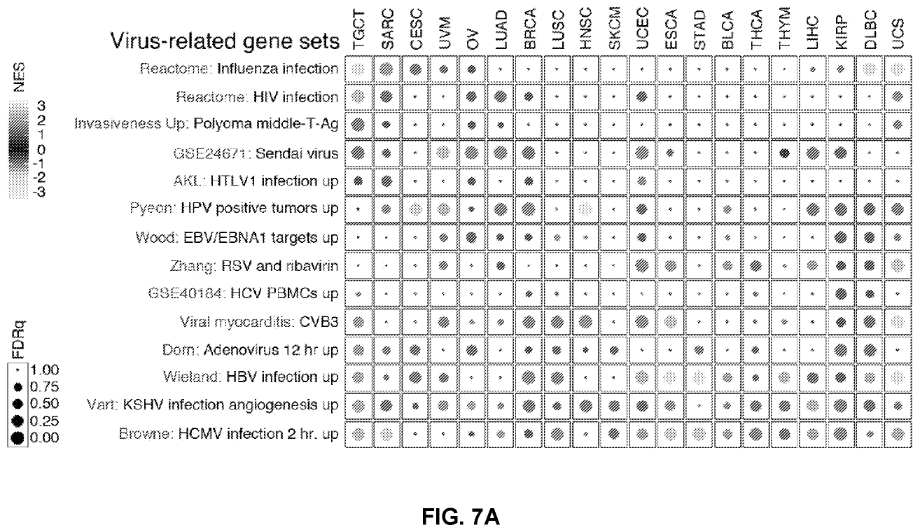

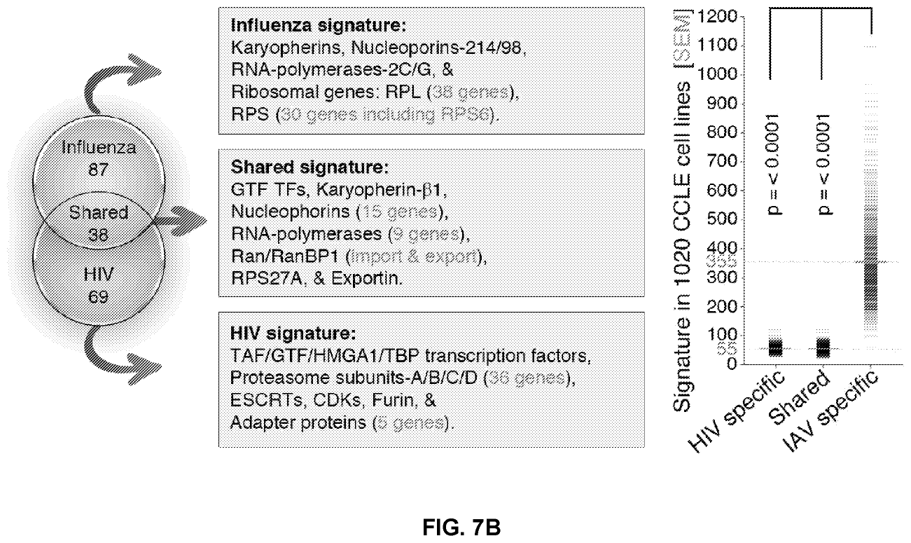

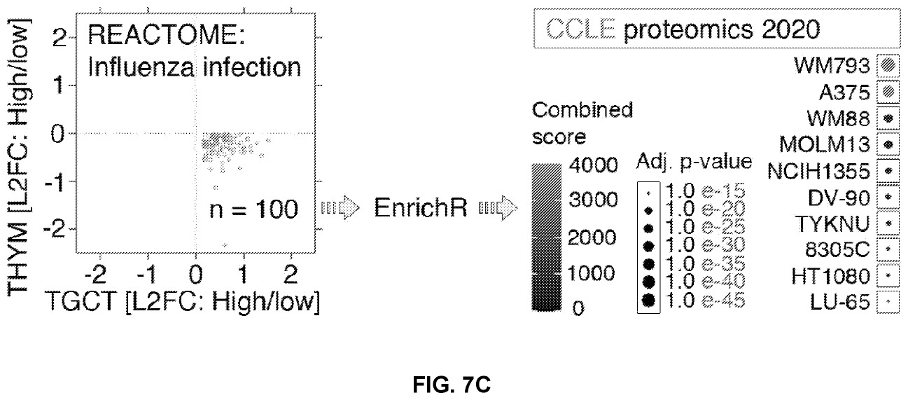

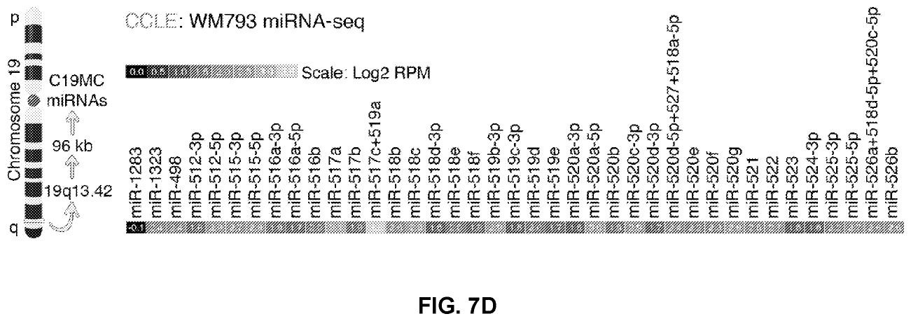

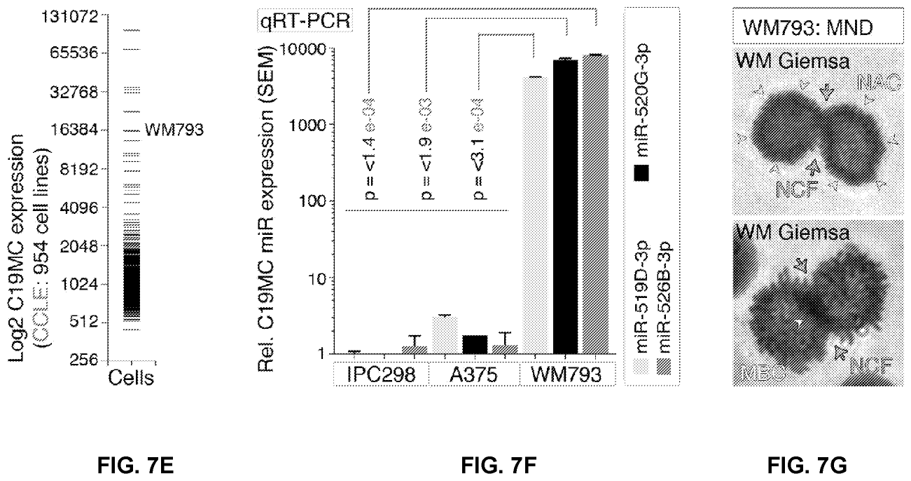

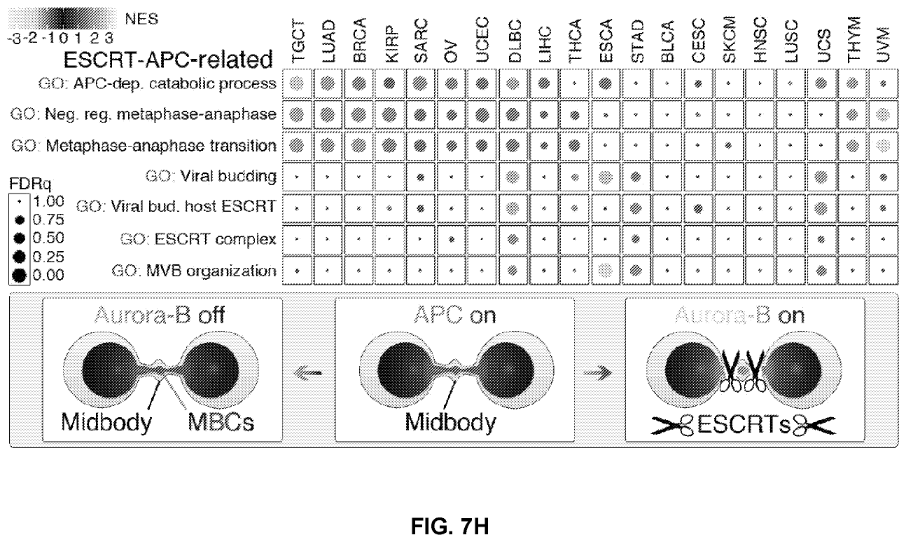

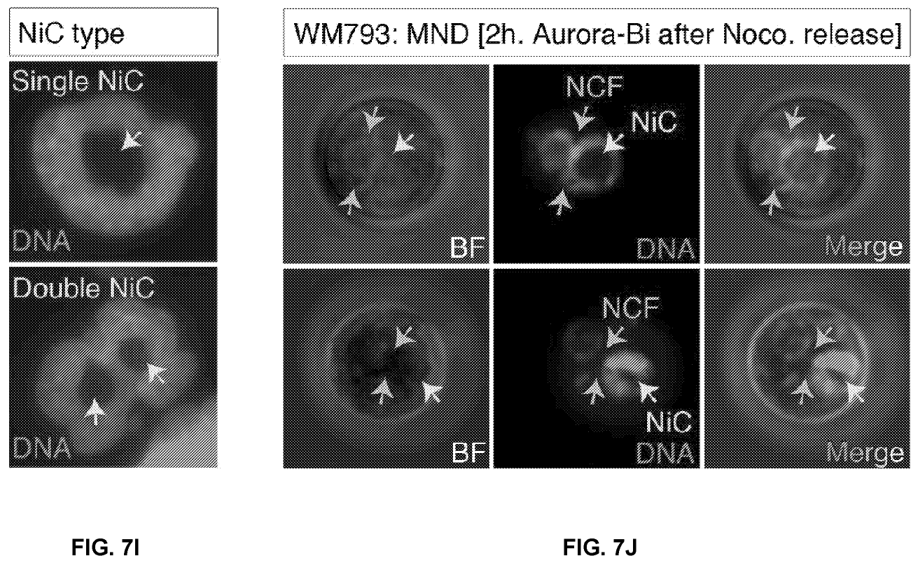

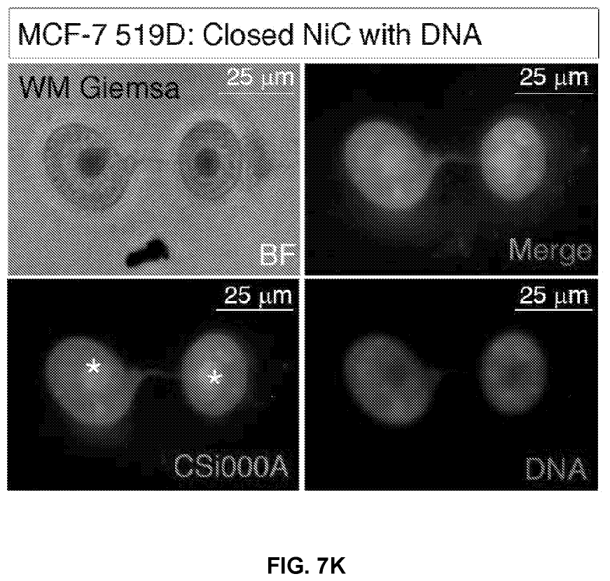

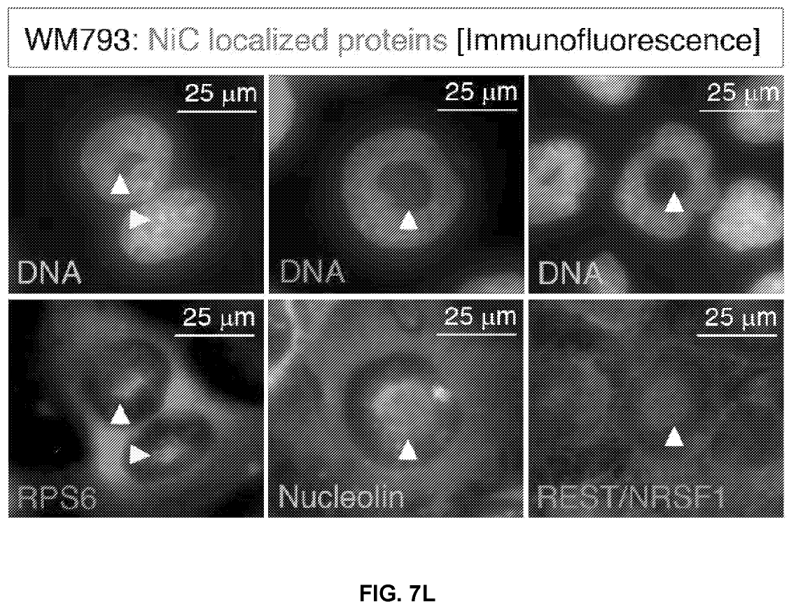

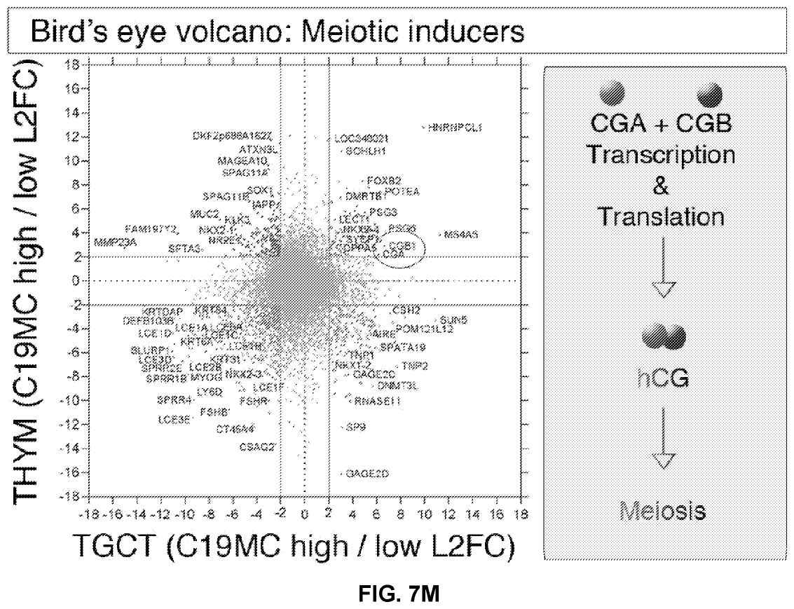

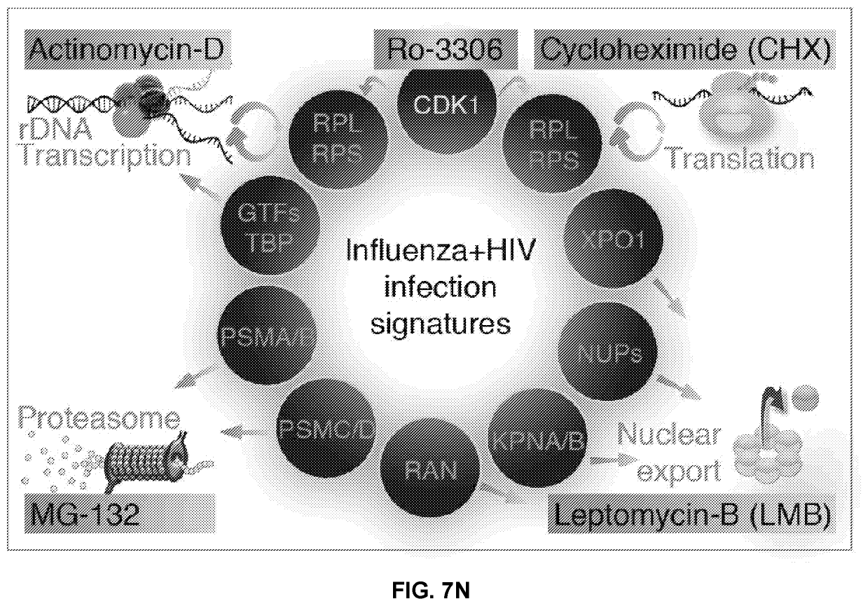

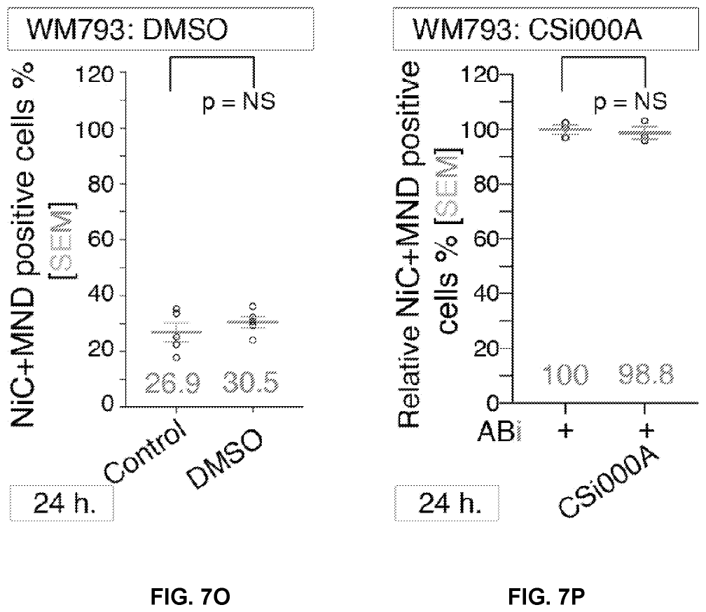

Certain signatures were tracked even though if it is not coherently enriched in C19MCHigh cancers as knowing its status was important to shed light on the biological context. For example, ESCRT versus APC signatures in FIG. 7H.
R code
>ggplot (DFname, aes(x=CancerTypeNES, y=Geneset))+
geom_point(aes(size=NegFDRq, color=NES))+
geom_point(shape=0, size=12)+
scale_color_gradientn(colours=c(“greenyellow”, “green2”, “black”, “red”, “yellow”),
limits=c(−3.5, 3.5),
breaks=c(−3.00, −2.00, −1.00, 0, 1.00, 2.00, 3.00))+
theme_classic( )
#Limits kept constant for all TCGA dot plots as it encompasses the data range of NES.

### CCLE 954 Cell Line Cumulative C19MC Expression Analysis

Cancer Cell Line Encyclopedia (CCLE) miRNASeq data were processed for cumulative expression of all 46 C19MC miRNAs (miR498, miR512-1, miR512-2, miR515-1, miR515-2, miR516A1, miR516A2, miR516B1, miR516B2, miR517A, miR517-B, miR517C, miR518A1, miR518A2, miR518B, miR518C, miR518D, miR518E, miR518F, miR519A1, miR519A2, miR519B, miR519C, miR519D, miR519E, miR520A, miR520B, miR520C, miR520D, miR520E, miR520F, miR520G, miR520H, miR521-1, miR521-2, miR522, miR523, miR524, miR525, miR526A1, miR526A2, miR526B, miR527, miR1283-1, miR1283-2, and miR1323) (Wickham, H. ggplot2: Elegant Graphics for Data Analysis. Springer New York, NY, 1-213 (2009)) and plotted in GraphPad Prism software to get an insight into the C19MC^{High} cell lines. Of note, WM793 is one of the top C19MC^{High} cell line (FIG. 7B).

### CCLE 1020 Cell Line IAV, HIV, and Shared Signature Analysis

Influenza-A virus (IAV)-infection signature and HIV-infection signatures were obtained from MSigDB. Both signatures were fed into GeneVenn to get unique (IAV-specific and HIV-specific) and shared genes of these signatures. Cancer Cell Line Encyclopedia (CCLE) RNAseq data were processed for IAV-specific, HIV-specific and shared gene set cumulative averages and plotted in GraphPad Prism software (FIG. 7B).

dbGAP Data Access and IGV Visualization of C19MC Transcriptional Landscape

Cancer Cell Line Encyclopedia (CCLE) RNAseq C19MC^{High} cell line data and NIH database of Genotypes and Phenotypes (dbGAP: The results published here are in part based upon data generated by The Cancer Genome Atlas managed by the NCI and NHGRI. TGCT C19MC^{High} patient RNAseq data BAM files were visualized for C19MC transcriptional landscape at C19MC locus using integrated Genomics Viewer (Jinesh, G. G., et al. PLoS One. 2018 18; 13(10):e0206008). While different transcriptional starts were noted, we focused on the start site at CpG86 location that matched WM793 cells. The zoom in feature was used to locate the precise start site, which matched to the CpG86 repeat sequence region. The CpG island ID: 86 was as per UCSC Genome Browser nomenclature (HG19).

### EnrichR Analyses

Critical gene signatures enriched in TCGA datasets such as meiosis and influenza signatures were filtered for enriched genes in C19MC^{High} tumors such as TGCT and fed into EnrichR (Nucleic Acids Res. 2016 44(W1):W90-7; Chen, E. Y. et al. BMC Bioinformatics. 2013 15:14:128; Xie, Z. et al. Curr Protoc. 2021 1(3):e90) to find matching cell lines either in NCI-60 cell line panel or in proteomics based CCLE database. Enrichments results such as adjusted p-value and combined score were plotted in R using ggplot2 package (R version 4.1.3).
R code
>ggplot(DFname, aes(x=Group, y=CellLine))+
geom_point(aes(size=AdjPvalue, color=CombinedScore))+
   geom_point(shape=0, size=10)+
scale_color gradientn(colours=c(“black”, “red”),
limits=c(0, 250))+
theme_classico
#Limits will change according to the data range.
   Bird's eye view volcano plot: meiotic-regulatory gene expression in C19MC^{High}groups of TGCT vs. THYM

To gain knowledge on the genes that are coregulated and differentially regulated in C19MC^{High} vs. C19MC^{Low} tumors of two prominent C19MC^{High} cancer types (TGCT vs. THYM) the RNAseq C19MC^{High} and C19MC^{Low} dataset genes were subjected to average and subsequent log 2 fold change (L2FC) calculations. The log 2 fold change data were subjected to XY volcano plotting in GraphPad Prism, with lines demarking the log 2 fold change level of 2 on each axis. The quadrant that represents genes that are upregulated in C19MC^{High} THYM as well as in C19MC^{High} TGCT were examined for meiotic regulatory genes. CGA and CGB1 corresponding to heterodimers of the meiotic-regulatory pregnancy hormone human chorionic gonadotrophin (hCG) (Tiwari, M. & Chaube, S. K. Dev Growth Differ. 2017 59(7):603-614) was marked in FIG. 7M.

### Cell Lines: Authentication, Quality Control and Maintenance

Human cancer cell lines were obtained, cultured at 37 °C with 5% CO₂ in MEM (Sigma: M4655)/RPMI 1640 (Hyclone #SH30255FS) supplemented with L-Glutamine (Gibco Life Technologies #A2916801), vitamins (Gibco Life Technologies #11120052), sodium pyruvate (Gibco Life Technologies #11360070), non-essential amino acids (NEAA: Gibco Life Technologies #11140050), penicillin-streptomycin (Gibco Life Technologies #15140122) with 10% FBS (Sigma #F0926) as described in the Table 1. The cells were DNA fingerprinted and periodically tested for Mycoplasma as described previously (Jinesh, G. G. et al. Sci Rep. 2020 10(1):12371; Jinesh, G. G. et al. Sci Rep. 2021 11(1):12673).

### Chemical Reagents/Inhibitors and Usage

A list of chemical agents used in this study are provided in the Table.2.

### Protein Hormone, Enzymes, Kits, Antibodies, Transfection Reagents and Usage

A list of protein and antibody reagents used in this study are provided in Table 3.

### Genetic Reagents (Anti-miRNAs, RNA-Probes)

A list of anti-miRs and RNA-probes used in this study are provided in Table 4.

### Genetic Reagents (Plasmid DNAs) and Stable Cell Lines Generated: No Viruses

A list of plasmids used in this study are provided in the Table 5. Of note, usage of viruses was averted throughout the study as the objective is to study the regulation and function of anti-viral response cluster C19MC in the absence of exogenous viruses.

### CRISPR Guide RNA Cloning and Sanger Sequencing

The C19MC upstream CpG86 repeat knock-out CRISPR guides (Choppers) were cloned as described (Ran, F. A. et al. Nat Protoc. 2013 8(11):2281-2308). Briefly, the Addgene plasmid pSpCas9(BB)-2A-Puro (PX459) V2.0 was digested with BbsI restriction enzyme to release the non-targeting part of the sgRNA, linear vector is gel purified, quantified, and custom synthesized and annealed oligo duplexes of chopper-1 and chopper-2 with suggested overhangs were ligated using Rapid ligation kit but using overnight incubation at 4 °C. The ligated plasmids were transformed into DH5a competent cells with SOC-medium recovery and plating as per standard procedures. Individual colonies were picked, screened by colony PCRs with parallel inoculation for glycerol stock preparation in Ampicillin-LB-broth. Positive colonies were subjected to plasmid-miniprep and Sanger sequencing (Genewiz/Azenta, USA) using individual “Colony PCR” primers (Table 6). Plasmids were confirmed for incorporation of oligonucleotide inserts and then subjected to plasmid MIDI-prep and quantified before transfections.

### CRISPR Genetic Engineering of CpG86

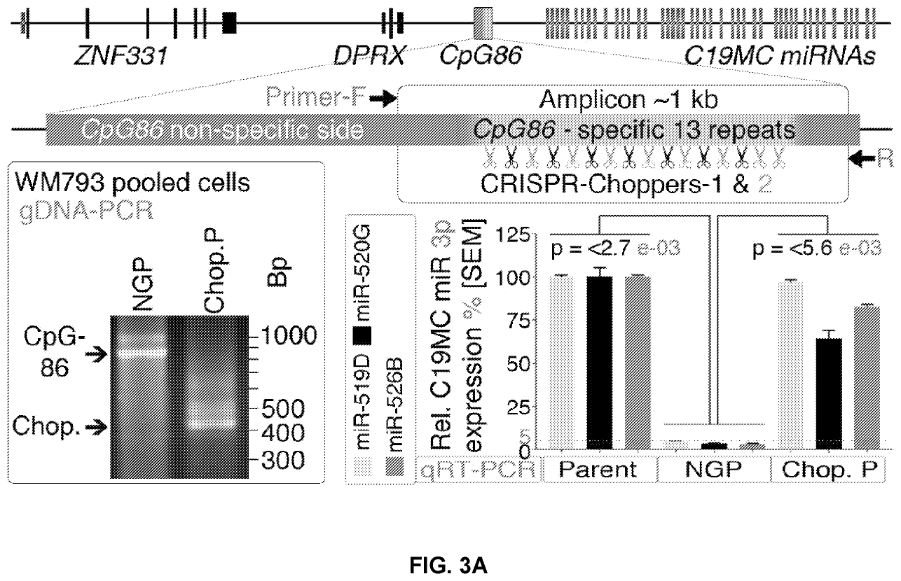

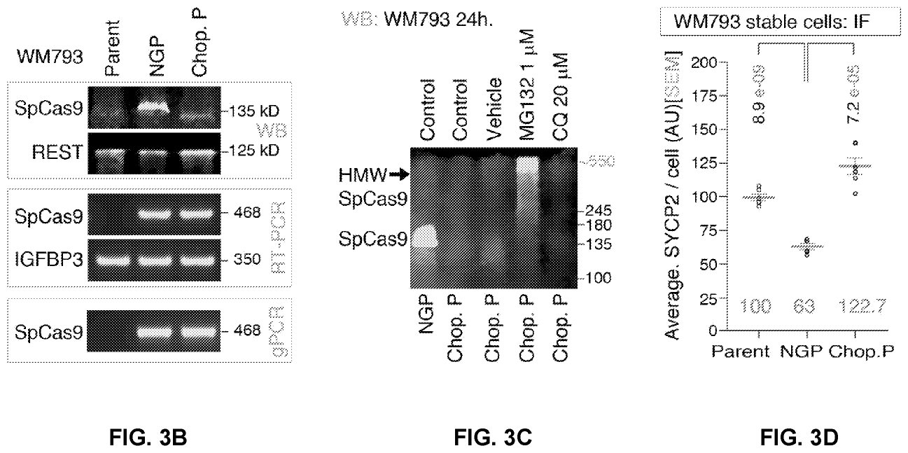

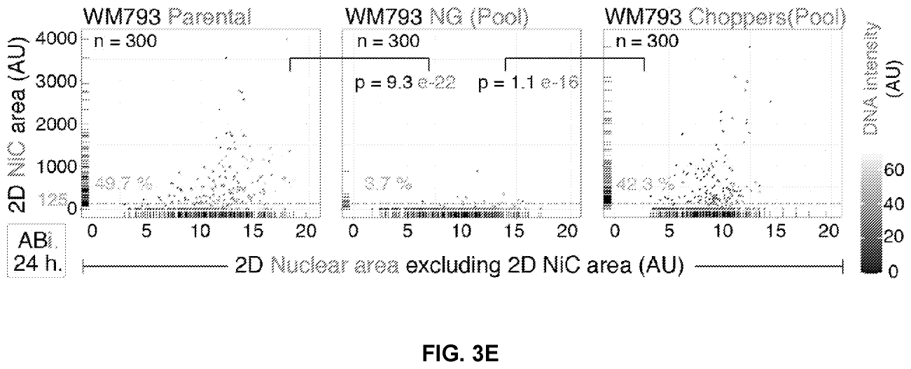

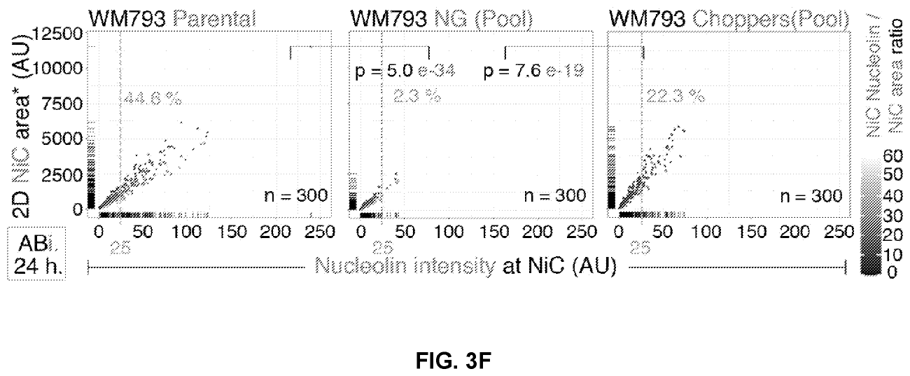

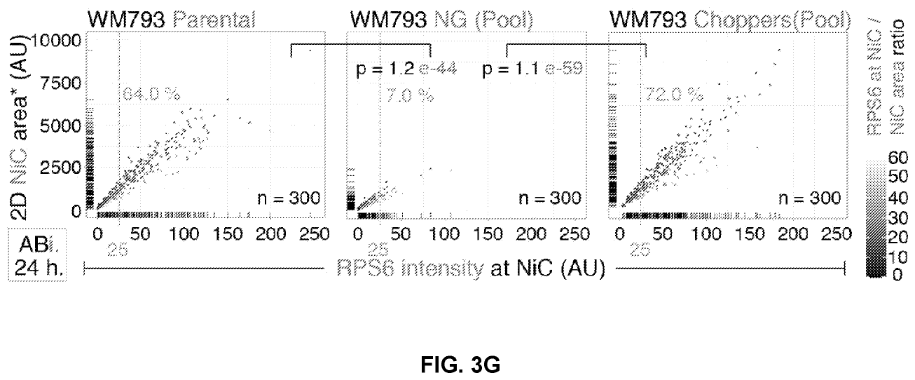

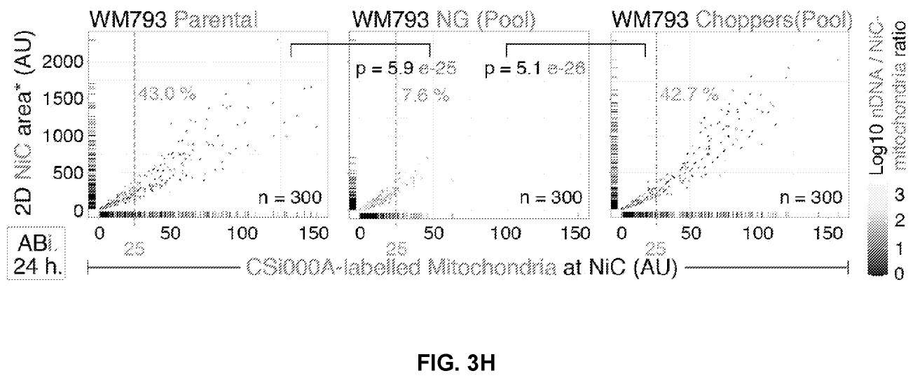

WM793 cells which has transcription start from the CpG86 13-repeat region through C19MC similar to C19MC^{High} TGCTs. Therefore, WM793 cells were transfected with pSpCas9(BB)-2A-Puro (PX459) V2.0 (has in-built SpCas9) or with CRISPR-choppers 1 and 2 constructs (have in-built SpCas9 plus chopper-guides targeting the 13-repeat regions of CpG86 for knock-out; FIG. 3A and Table 5) using Lipofectamine-2000 as per manufacturer's protocol with minor modifications (See transient transfection section below). Parental WM793 cells were maintained in parallel to have it as a control. Transfected cells were stably selected using gradual increase of puromycin from 0.5 ρg/ml to 4 μg/ml. Upon reaching confluency in transfected cells, the cells were trypsinized and plated for genomic DNA isolation and freezing. Genomic DNA was isolated from parental cells, NGP (Nontargeting-Guide-sgRNA pooled stable cells), and Choppers (Chopper-1 and 2 co-transfected stable pooled cells) and quantified using Nanodrop. 150 ng of genomic DNAs were subjected to CRISPR-screening PCR using “Chop86” primers (Table 6) and the laddering of amplicons in choppers with size <999 were confirmed to have undergone repeat deletions and fusion of flanking ends, as the primers bind outside the 13-repeat region.

### Sanger Sequencing of CRISPRed Genomic Regions:

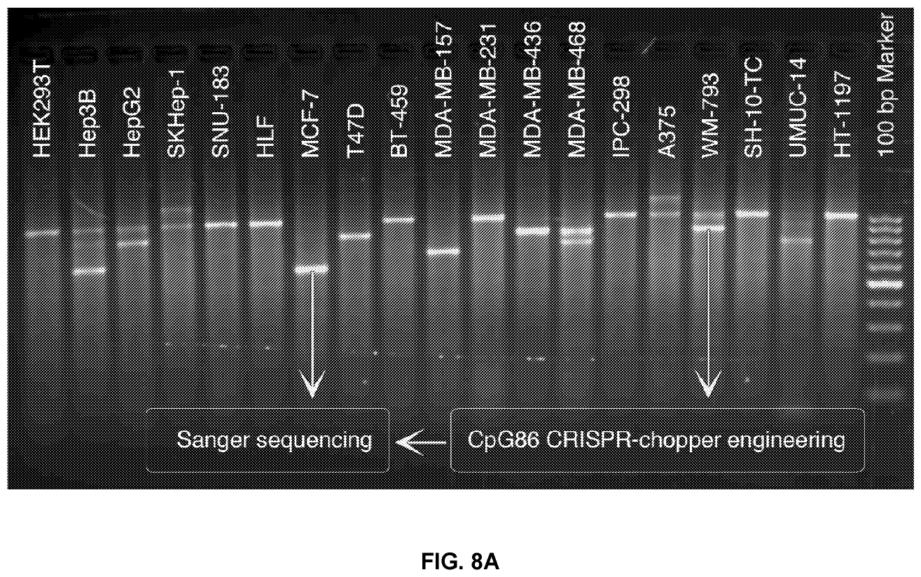

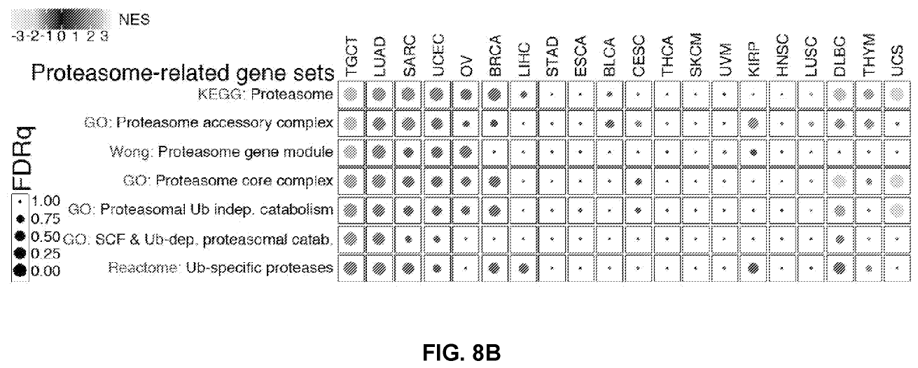

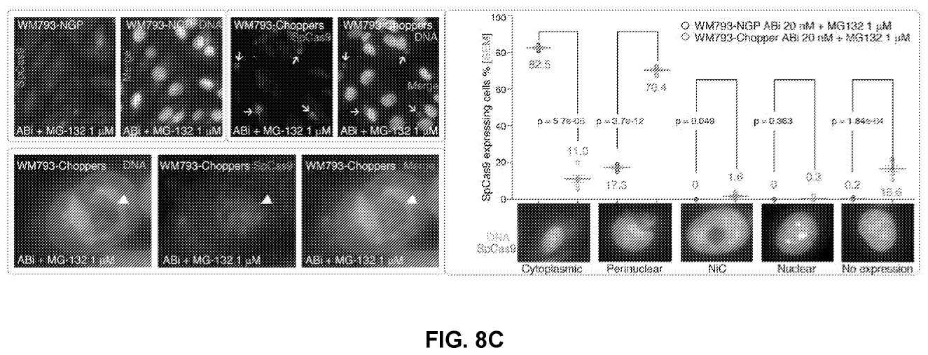

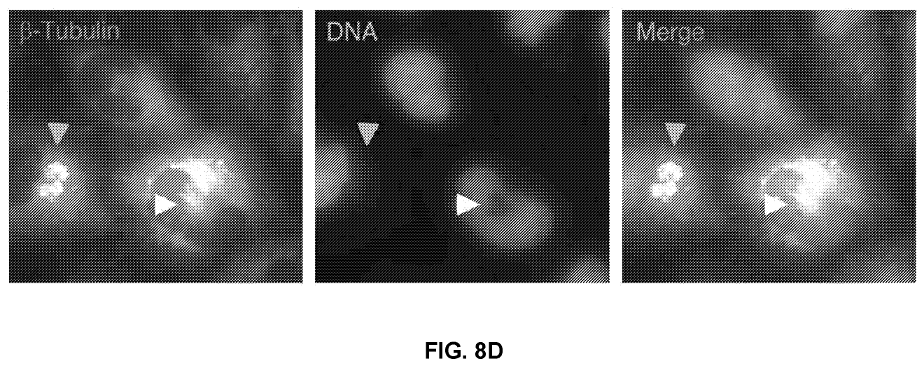

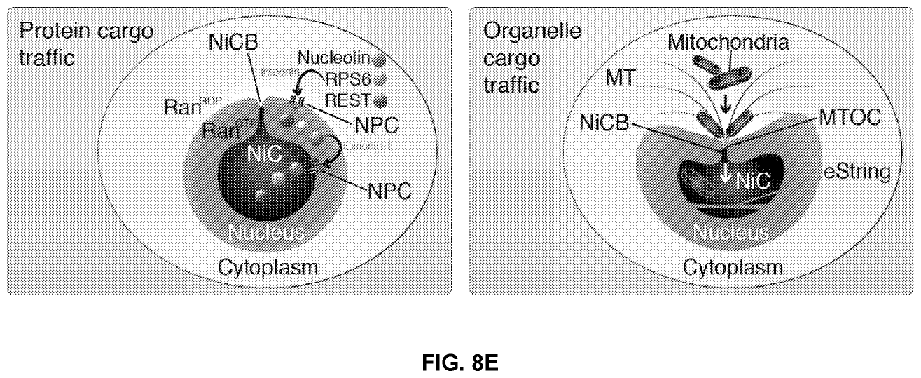

To confirm that the CRISPR-choppers indeed deleted the repeat region, Chop86 primer (Table 6)-directed DNA amplicons from genomic DNA of WM793-chopper cells were subjected to TA-cloning in pGEM-T Easy vector system and minipreps of plasmids were subjected to Sanger sequencing (Genewiz/Azenta, USA) using individual “Chop86” primers (Table 6). We confirmed that, the choppers-1 and 2 resulted in the knock-out of 10 out of 13 repeats within the CpG86 upstream of C19MC (FIGS. 3A, 8B).

### CpG86: 13-Repeat Hypervariability Test

To examine the repeat numbers within CpG86 (UCSC Genome browser-HG19) that is upstream of C19MC across multiple human cancer cell lines, Chop86 primer (Table 6)-directed DNA amplicons from genomic DNA of 19 cancer cell lines from various cancer type were subjected to PCR. The cell lines exhibited hypervariability in this region as evidenced by high variability in product size. As it appeared MCF-7 cells had naturally smallest amplicon among all products and is of in meiotic interest, we subjected the MCF-7 amplicon by Sanger sequencing (Genewiz/Azenta, USA) using individual “Chop86” primers (Table 6). We confirmed that, MCF-7 cells naturally lack 8 out of 13 repeats within the CpG86 upstream of C19MC (FIG. 3A, 8B).

### Genomic DNA CRISPR-Screening PCRs, Colony PCRs and Primers:

A list of PCR primers used in this study and their reaction conditions are provided in the Table 6. All PCR reactions were performed with an initial denaturation of 95 °C for 3 minutes; cycling conditions of 95 °C for 1 minute, 60 °C for 30 seconds, 72 °C for 1 minute for 34 cycles; and a final extension time of 72 °C for 5 minutes. All reactions had a final concentration of 1M betaine.

### Transient Gene/Anti-miR Transfections

WM793 cells in 6-well plates (50,000 cells/well, plated 24 hours before) were transfected with 1 g of pEGFP-C1-EMD (specified in Table 5) using Lipofectamine 2000 as per manufacturer's protocol with minor modifications. Briefly, 300 μl of Opti-MEM was mixed with 5 μl of Lipofectamine 2000 and separately, 300 μl of Opti-MEM was mixed with 1 μg of pEGFP-C1-EMD plasmid and incubated for 5 minutes. Both mixtures were pooled by gradual mixing and incubated further in room temperature for 30-40 minutes. This approximately 600 μl mixture was added to the media removed cells in a drop-wise fashion, and further 3.5 ml complete MEM was added gently to the cells/well of 6-well plate. 24 hours after, the media was changed again. 48 hours after transfection, cells were imaged for EGFP-emerin expression with or without live nuclear DNA staining using Hoechst-33342.

All anti-miR transfections were done similar to emerin transfections, but using RNAiMAX reagent instead of Lipofectamine 2000, and plasmids were substituted with 30 nM anti-miRs. Cy5 labelled non-targeting RNA was used to monitor transfection efficiency. 24 hours after transfection, the media was changed with or without treatments as per experiment set-up.

For pMRX-IP-GFP-LC3-RFP plasmid transfection, please see below at section: Detection of autophagic flux within sub-cellular locations.

Stable Transfections of Single C19MC miRNAs and Cell Sorting

MCF7 cells in 6-well plates were transfected with 1 μg of pMIR, pMIR-519D and pMIR-526B (viral transduction was averted throughout the study as C19MC is a viral response cluster) using Lipofectamine 2000 as described previously (Jinesh, G. G. et al. Sci Rep. 2021 11(1):12673). 48 hours after transfection the cells were selected against gradual doses of puromycin (0.5, 1, 2, and 4 g/ml each subsequent week) for a period of 2 months, expanded and were sorted (BD FACSAria Cell Sorter) for GFP, and further expanded GFP-positive cells before freezing. The cells were maintained in 4 μg/ml puromycin containing MEM and were switched to puromycin free medium for experiments.

### Microscopy

Microscopy was performed as described previously (Jinesh, G. G. et al. Sci Rep. 2020 10(1):12371; Jinesh, G. G. et al. Sci Rep. 2021 11(1):12673). Cells were imaged using Zeiss Observer.Z1 microscope equipped with Axiocam 503 mono (Zeiss) camera. For fluorescence microscopy the individual channel images were pseudo-colored if indicated in figures/legends or imaged in grey scale (for Giemsa cytogenetics), merged, and exported to jpeg using ZEN 2.3 Pro software (Carl Zeiss Microscopy, GmbH, 2011, Blue edition). The final composite was created using Adobe Photoshop CS5 (Adobe Systems Inc., San Jose, CA, USA).

### Nucle(ol)ar Invasion of Cytoplasmic-Materials [NiC] Assays

WM793 cells that constitutively overexpress most of 46 C19MC miRNAs were treated with 20 nM Aurora-B inhibitor (Aurora-Bi: AZD1152-HQPA) or DMSO for 24 hours and were stained for DNA using Hoechst-33342 for 20 minutes before imaging the cells for Nucle(ol)ar invasion of cytoplasmic-material by evaluating the appearance of large Hoechst-33342-positive-DNA-free area within nuclei. The nuclear DNA stain images were subjected to NiC 2D area measurement using ImageJ software. The arbitrary area units obtained in ImageJ were converted to square micrometer area unit using a reference scale box of 100 sq. mm. For some experiments arbitrary units were used as such or after down-scaling. Three hundred cells (nuclei) from replicates were counted/measured for NiC area/positivity and were plotted in GraphPad Prism software using aligned dot plot (barcode symbol) using the settings of standard error of mean. The data points were displayed with 50% transparency to visualize the overlap density.

To evaluate changes in the size of NiC, meiosis promoters hCG (Prasad, S., et al. Dev Growth Differ. 2016 58(3):293-302) (25 ng/ml), all-trans retinoic acid (Pellegrini, M. et al. Cell Cycle. 2008 7(24):3878-88) (ATRA:1 μM), and non-lethal doses of IAV+HIV signature inhibitors (FIG. 7N) such as actinomycin-D, cycloheximide, MG-132, leptomycin-B, Ro3306 were treated in WM793 cells with or without 20 nM Aurora-B inhibitor (AZD1152-HQPA) for 24 hours, and were stained for DNA using Hoechst-33342 for 20 minutes before imaging the cells. The counting and or measurement of NiC area were done as described above.

To evaluate the relationship of nuclear DNA replication, NiC area and intensity were measured in addition to Hoechst-33342-positive DNA area/intensity (Nuclear area/intensity subtracted with NiC area/intensity).

To evaluate the requirement of C19MC to form NiC, CRISPR-engineered WM793 pooled cells were used with Aurora-B inhibitor (AZD1152-HQPA) for 24 hours and were stained for DNA using Hoechst-33342 for 20 minutes before imaging the cells. The use of pooled cells eliminates the possibility of clonal effect/drift in NiC phenotype. NiC area versus nuclear area and DNA intensity results were plotted in R using ggplot2 package (R version 4.1.3). Nuclear area measurements were scaled down by dividing the AU values by 1000 (FIG. 3E). T-tests were done in Microsoft Excel to calculate the significance.
R code
>ggplot(DFname, aes(x=MeasurementsNucArea, y=MeasurementsNiCArea))+
geom_point(aes(colour=MeasurementsDNAintensity))+
scale_color_gradientn(colours=c(“black”, “red”, “yellow”),
limits=c(0, 70))+
   xlim(0, 20)+
   ylim(0, 4000)+
geom_rug( )+
theme_bw( )
#Limits kept constant according to the data range of all three cell types.
#Axis limits set based on the data range of all three cell types combined.

### Detection of Mitochondria Within NiC

WM793 cells were plated in 6-well plates (50,000 cells/ml; 4 ml/well) and 24 hours after treated with 20 nM Aurora-B inhibitor (AZD1152-HQPA) for 24 hours. 30 minutes before the end of treatment duration, the cells were live stained with JC-1 and Hoechst-33342 before imaging using Zeiss Observer.Z1 microscope. Polarized mitochondria, and DNA were imaged using appropriate filters for red, and blue channels respectively and pseudo-colored as indicated in figures.

Independently, WM793 cells were transfected with 30 nM Cy5-labelled CSi000A RNA probe and 24 hours after transfection, treated with 20 nM Aurora-B inhibitor (AZD1152-HQPA) for 24 hours. 30 minutes before the end of treatment duration, the cells were live stained with Hoechst-33342 before imaging using Zeiss Observer.Z1 microscope. Cy5-CSi000A-RNA-labelled mitochondria, and DNA were imaged using appropriate filters and pseudo-colored as indicated in figures.

Detecting Giemsa Positive DNA, and Mitochondria within the Closed NiC Compartment

To understand NiC is a closed compartment or a result of donut like nucleus we decided to isolate the nuclei by hypotonic swelling where cytoplasmic materials will be lost but the nuclear integrity can be retained by restricting the swelling time limited. We combined this strategy to also explore the presence of DNA and mitochondria within the NiC compartment. Briefly, MCF-7-miR-519D stably transfected cells were plated at a density of 100,000 cells/ml for 24 hours and treated with 20 nM Aurora-B inhibitor for 24 hours in 10% FBS containing MEM. The cells were collected by shake-off plus vigorous pipetting to dislodge cells. The cells were pelleted at 1000 rpm and resuspended with 7 mL of 0.067 mol/L KCl, allowed to swell for 1 minute, fixed (two step: 7 drops and then 7 mL of fresh fixative 3:1 methanol: glacial acetic acid). The cells in fixative were incubated at −20 °C overnight, washed with fresh fixative, dropped-in on “+ slides”, air-dried, aged for 1 hour at 70 °C using a slide warmer (Fisher Scientific), rehydrated and dehydrated using graded-series of ethanol, and stained with 2% modified Giemsa stain (PBS, 70% ethanol, 95% ethanol, 3 dips each, air-dried, 2% Wright's modified Giemsa staining for 5 minutes, 3 distilled water dips and air-dried). The slides were then incubated with 30 nM Cy-5-labelled-CSi000A+10 nM Hoechst-33342 for 20 minutes at room temperature, and washed with PBS three times. Imaging was done using appropriate filters for Hoechst-33342, Cy-5 or bright field, and image processing was done as described in microscopy section. NiC was found positive for Giemsa-positive DNA, CSi000A-positive mitochondria and relatively free from Hoechst-33342-positive DNA (FIG. 7K).

### Quantification of Mitochondria Traffic in to the NiC Compartment and Visualization in R

WM793 parental cells, WM793-NGP cells (CRISPR-control with SpCas9 stably transfected), and WM793-CRISPR-CpG86-engineered chopper cells (Choppers-1 and 2 with SpCas9 stably transfected) were plated in puromycin free complete MEM at a density of 50,000 cells/ml; 4 ml/well of 6-well plate for 24 hours. The cells were then treated with 20 nM Aurora-B inhibitor (AZD1152-HQPA) and 30 nM CSi000A (not using transfection reagent) for 24 hours and protected from light from this step onwards. 30 minutes before the end of treatment duration, the cells were live stained with Hoechst-33342 before imaging using Zeiss Observer.Z1 microscope. Exposure times for each channel were kept constant between conditions. Hoechst-33342 and CSi000A channels were exported individually as well as merged before subjected to quantification of nuclear DNA and mitochondria within NiC using ImageJ. While most of WM793-NGP cells do not form NiC, a small free-hand tool dot was drawn (<100 arbitrary unit [AU] in FIG. 3H) inside nuclei to get a measurement for a data point. To nullify this in CSi000A-labelled mitochondria at NiC, we introduced a generous gate at 25 scaled down AU (Original AU is <250) on X-axis (dotted line in FIG. 3H) and compared the mitochondria influx over 25 scaled down AU between CRISPR-CpG86-engineered cells versus NGP or parental cells. A log 10 ratio of nuclear DNA (Hoechst-33342 positive) to CSi000A-labelled mitochondria at NiC were calculated to evaluate the relationship of nuclear DNA content to NiC invasion of mitochondria. The CSi000A intensity was scaled down by dividing the intensity AU values by 10. Mitochondria traffic into NiC results were plotted in R using ggplot2 package (R version 4.1.3). The gate was introduced in Adobe Photoshop CS5, and the percentage calculations based on gate values were done using Microsoft Excel. T-tests were done in Microsoft Excel to calculate the significance.
R code
>ggplot(DFname, aes(x=CSi000AintensityAtNiC, y=NiCAreaAU))+
geom_point(aes(colour=L10nDNAtoNiCCSiO00ARatio))+
scale_color_gradientn(colours=c(“black”, “red”, “olivedrab1”, “yellow”),
limits=c(0, 3.5))+
geom_rug( )+
theme_bw( )
#Limits kept constant according to the data range of all three cell types.

### Immunofluorescence

SYCP2: Immunofluorescence was performed as described previously (Jinesh, G. G. et al. Cell Death Discov. 2016 2:16003; Jinesh, G. G. et al. Cell Death Discov. 2016 2:16003; Jinesh, G. G., et al. Biochem J. 2016 473(1):99-107). Briefly, stable MCF-7-pMIR, MCF-7-miR-519D, MCF-7-miR-526B (GFP expressing), WM793 parental cells, CRISPR-Cas9 control-NGP pooled cells, and CRISPR-CpG86-engineered WM793 chopper pooled cells were plated in 24 well plates at 50,000 cells/ml and cultured for 48 hrs with a media change at 24 hrs. The cells were fixed in ice cold methanol at −20 °C for a period of 24-48 hrs. The cells were then washed with PBS, blocked for 30 minutes at room temperature using 300 μl blocking buffer/well (1% BSA and 0.3% Triton-X100 in PBS). The cells were then incubated overnight with SYCP2 antibody (Sigma, HPA062401: Dilution 1:250) in blocking buffer. The cells were washed and incubated with F(ab)2-goat anti-rabbit IgG (H+L) labelled with Alexa fluor-555 or Cy5 (1:1000 dilution in blocking buffer) for 1 hour at room temperature while protected from light. The cells were washed with PBS thrice and incubated with 10 nM Hoechst-33342 in PBS (Sigma) for 20 minutes, and imaged. Exposure times for each channel were kept constant between conditions. The total fluorescence signal per frame was quantified using ImageJ and the number of cells were counted to calculate average SYCP2 intensity/cell/frame. Six such random frames were quantified and the data were plotted as aligned dot plot with standard error of mean in GraphPad Prism software.

β-Tubulin: Immunofluorescence for β-Tubulin was performed similarly in WM793 cells treated with 20 nM Aurora-B inhibitor for 24 hours (to generate NiC), but with goat anti-mouse IgG (H+L) cross-adsorbed secondary antibody labelled with Cyanine-5 (1:1000 dilution in blocking buffer). Quantification was not performed as >90% of cells lacked β-Tubulin expression except small clusters of cells. Therefore, MTOC positive for β-Tubulin invasion into NiC is qualitative (FIG. 8C).

Nucleolin/C23, RPS6, REST/NRSF: Please see the section: Quantification of nucleolin, RPS6, REST/NRSF traffic within NiC and visualization in R.

Quantification of Nucleolin, RPS6, REST/NRSF Traffic into the NiC Compartment and Visualization in R

WM793 cells or CRISPR-engineered WM793 cells in parallel to WM793 parental cells were treated with or without Aurora-B inhibitor and or CDK1 inhibitor Ro3306 (as indicated in the figures) for 24 hours, fixed and processed for immunofluorescence of nucleolin, RPS6, REST/NRSF as described above with appropriate Cy-5 labelled secondary antibodies. The cells after final wash were incubated with 10 nM Hoechst-33342 in PBS for 20 minutes, and imaged. The immunofluorescence signals within NiC regions were marked with the aid of Hoechst-33342 DNA image and quantified for area and intensity per NiC/cell. Of note, a small pool of RPS6 and nucleolin are always present within nuclei as part of original nucleolus and ribosome biogenesis process, even in the absence of visible NiC formation (Hoechst-33342 DNA-free regions within nucleus). To nullify this pool, a gate was applied (green dotted line) that matches to the baseline pool of nucleolin and RPS6 at conditions that do not form NiC (WM793-NGP cells) (FIG. 3F-3G). Exposure times for each channel were kept constant between conditions. The NiC immunofluorescence data from 300 individual cells for each condition were quantified in addition to area, and were plotted in R using ggplot2 package (R version 4.1.3). The gates were introduced in Adobe Photoshop CS5, and the percentage calculations based on gate values were done using Microsoft Excel.
R code
>ggplot(DFname, aes(x=NiCimmunofluorescence, y=NiCAreaAU))+
geom_point(aes(colour=NiCintensityVsAreaRatio))+
scale_color_gradientn(colours=c(“black”, “red”, “yellow”),
limits=c(0, 60))+
   xlim(0, 250)+
   ylim(0, 10000)+
geom_rug( )+
theme_bw( )
#Limits kept constant according to the data range of all three cell types.
#Axis limits were based on the range of measurements in all three cell types.

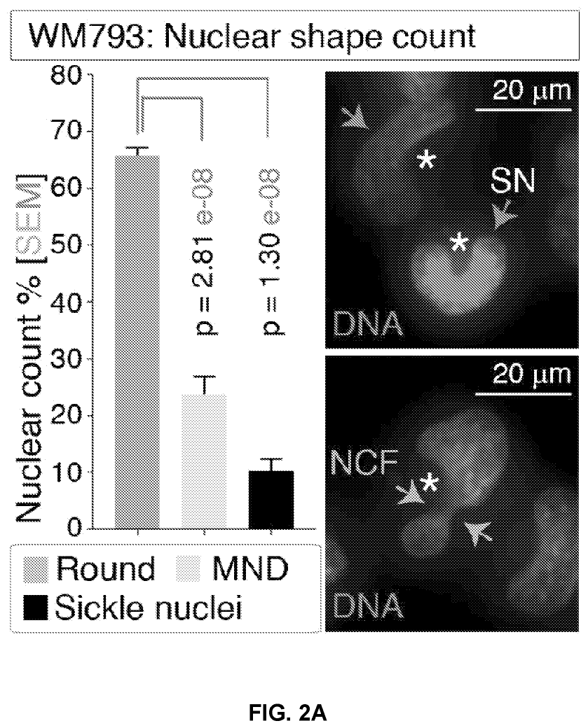

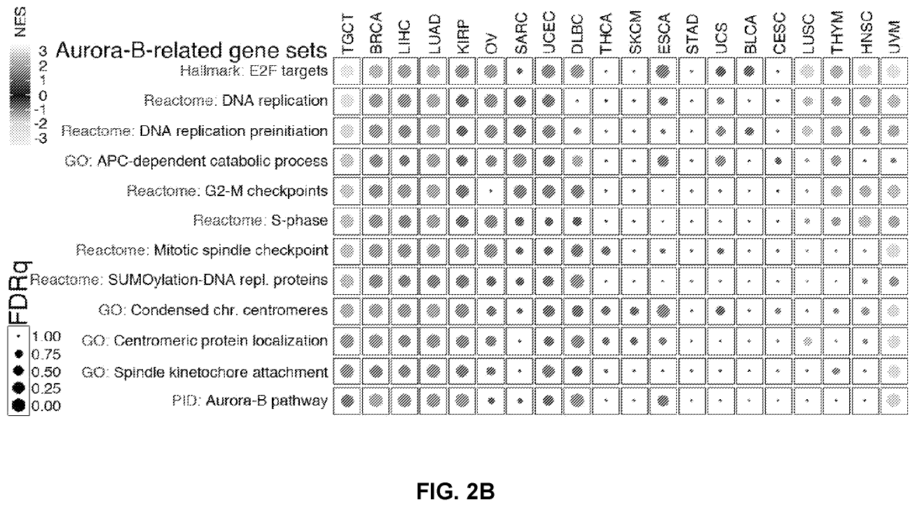

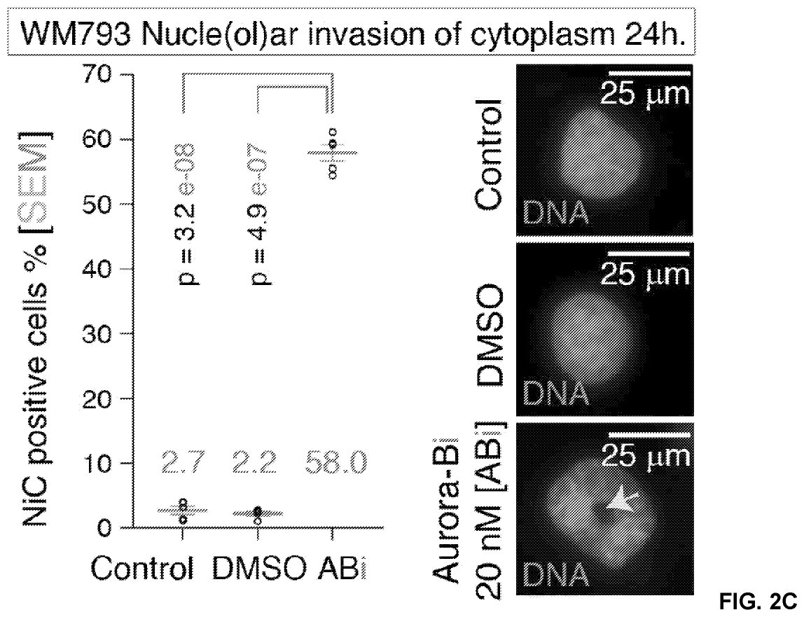

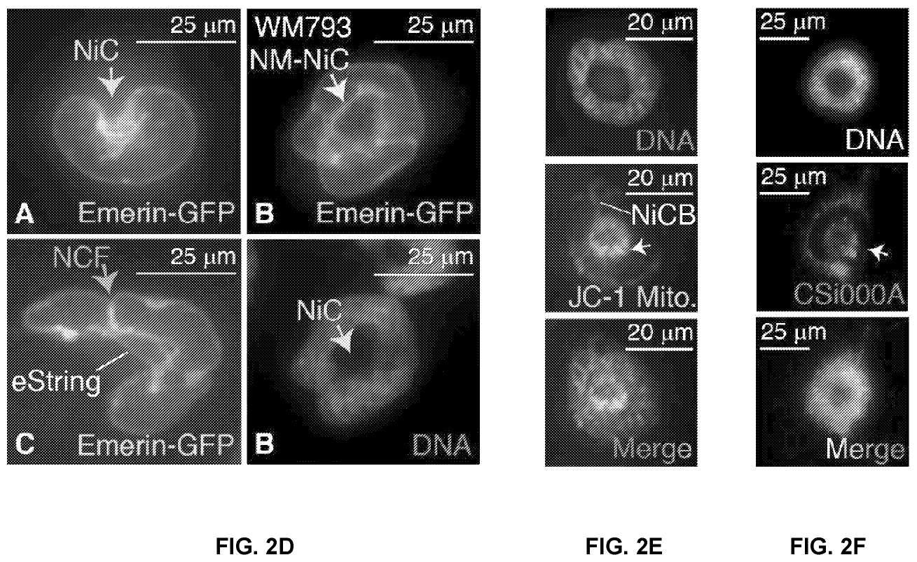

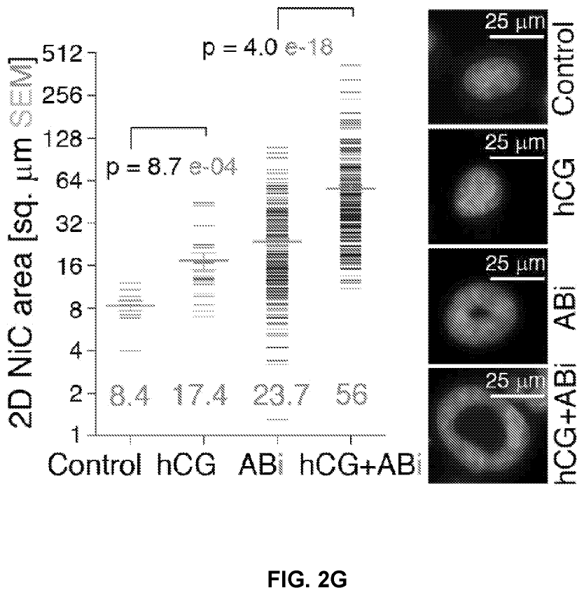

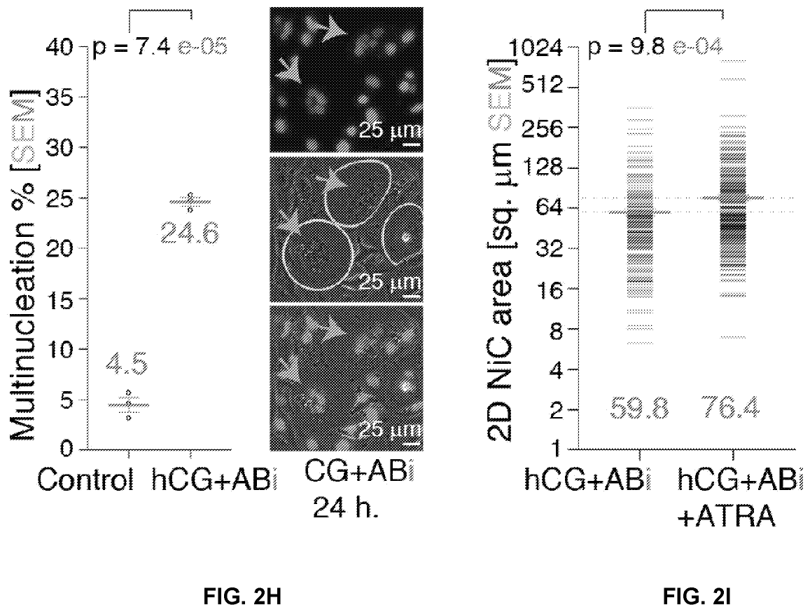

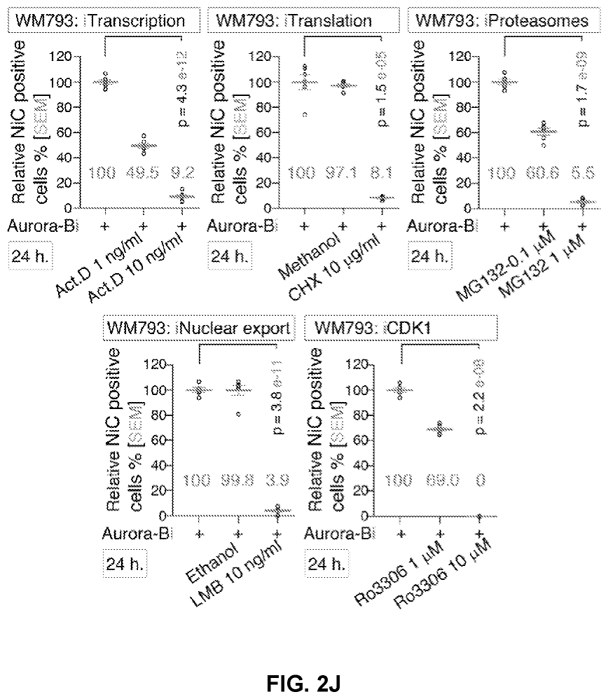

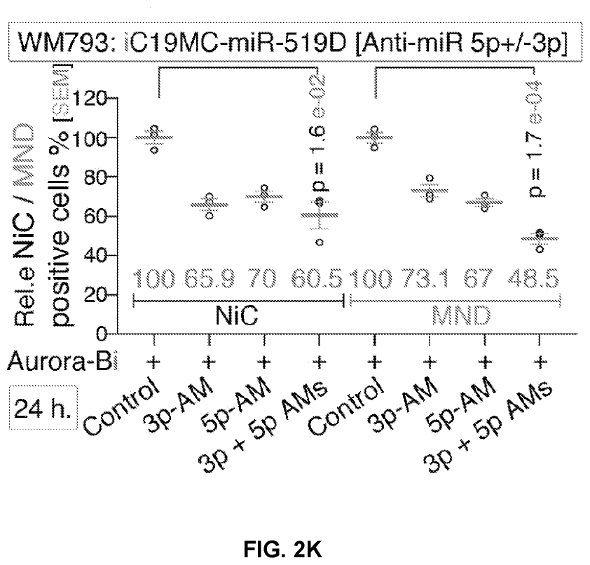

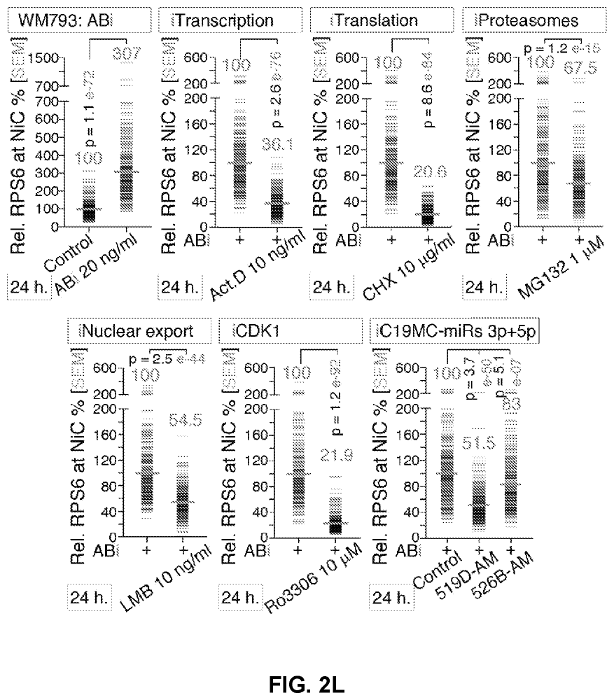

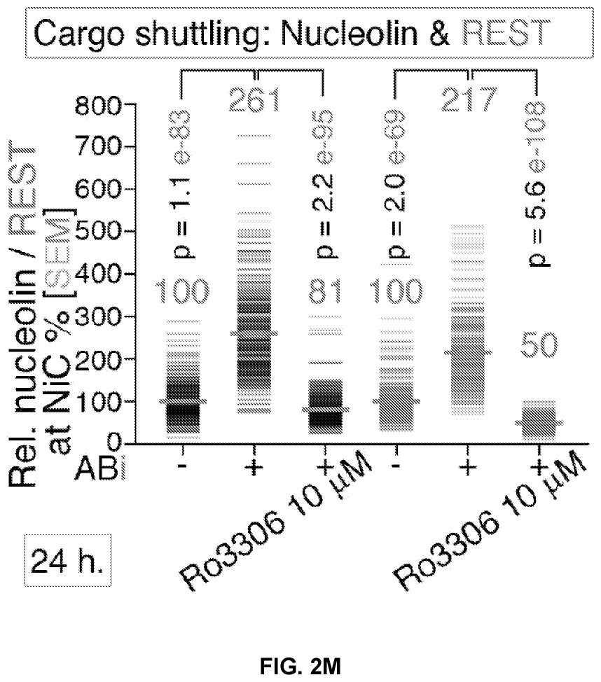

Alternatively, the data were plotted in GraphPad Prism software as aligned dot plot with standard error of mean. Data points were given 75% transparency to show the data dense regions (FIG. 2L-2M). T-tests were done in Microsoft Excel to calculate the significance.

Detection of Autophagic Flux within Sub-Cellular Locations (Cytoplasmic Vs. NiC)

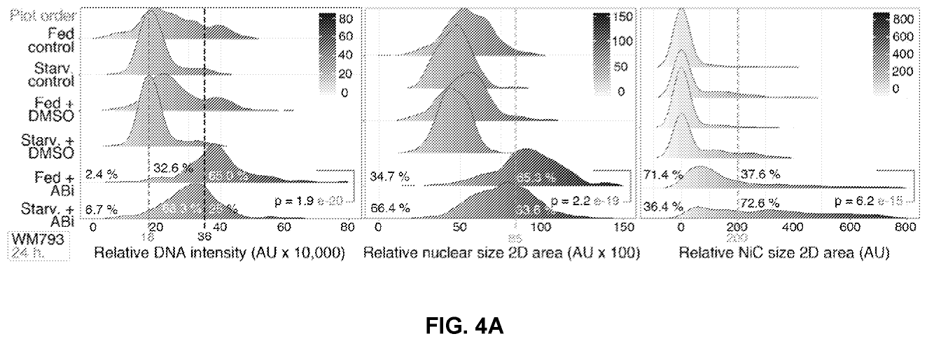

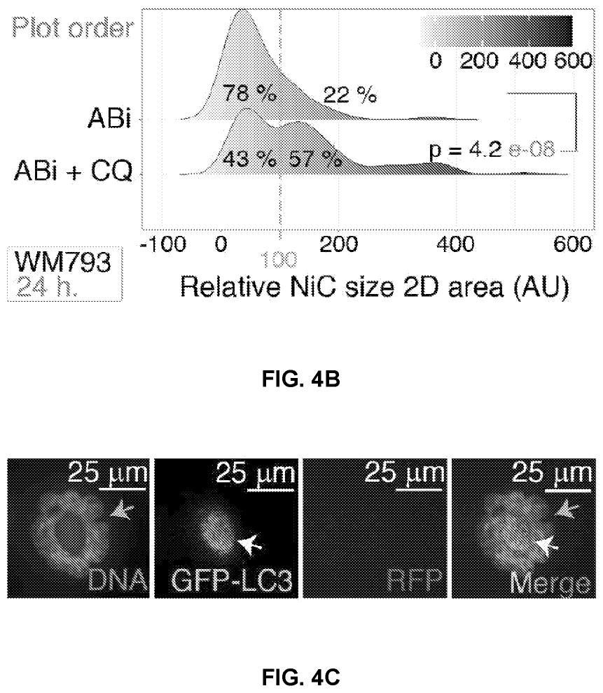

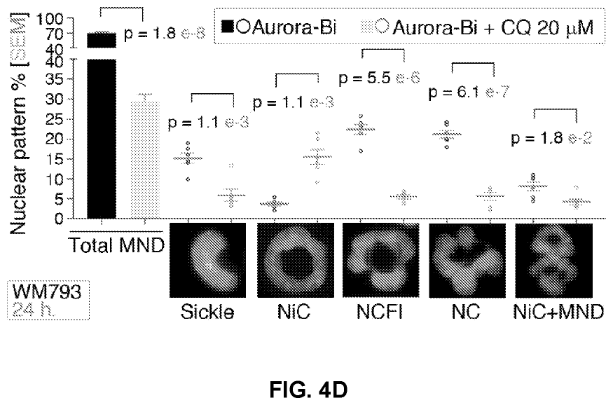

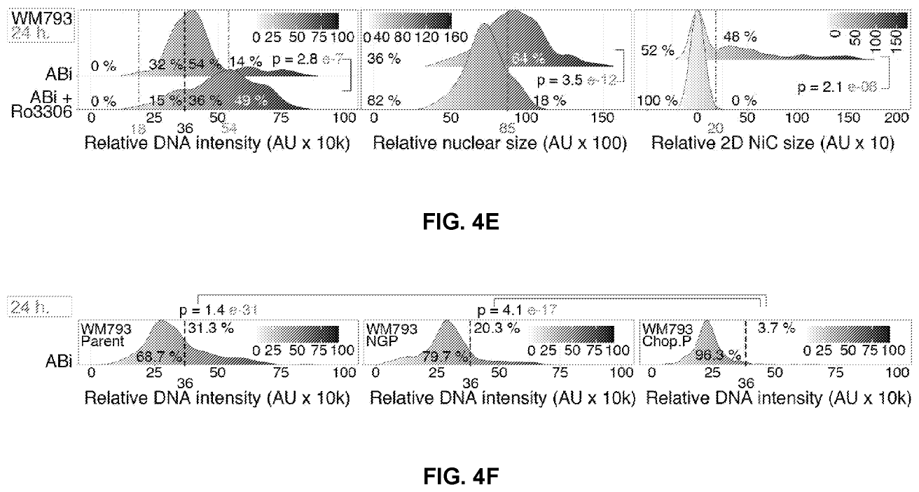

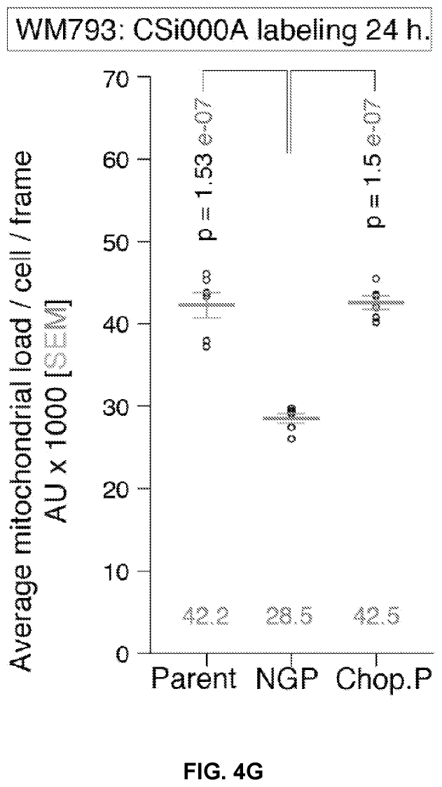

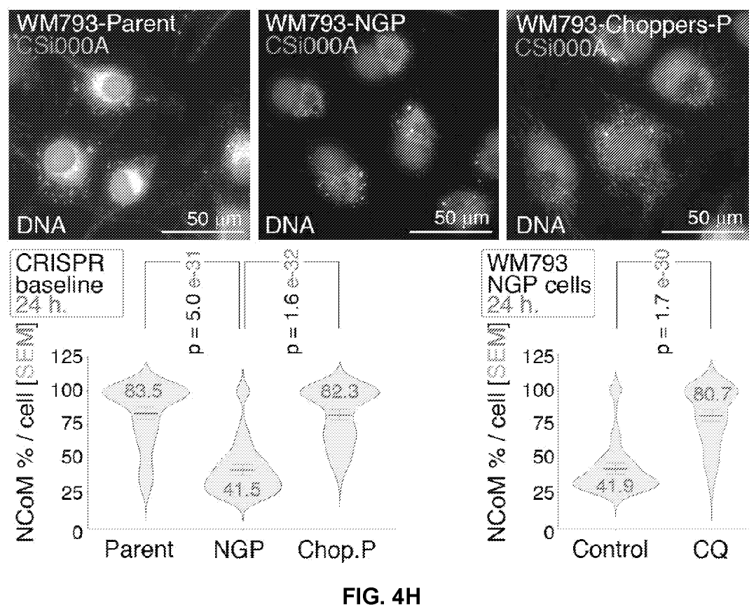

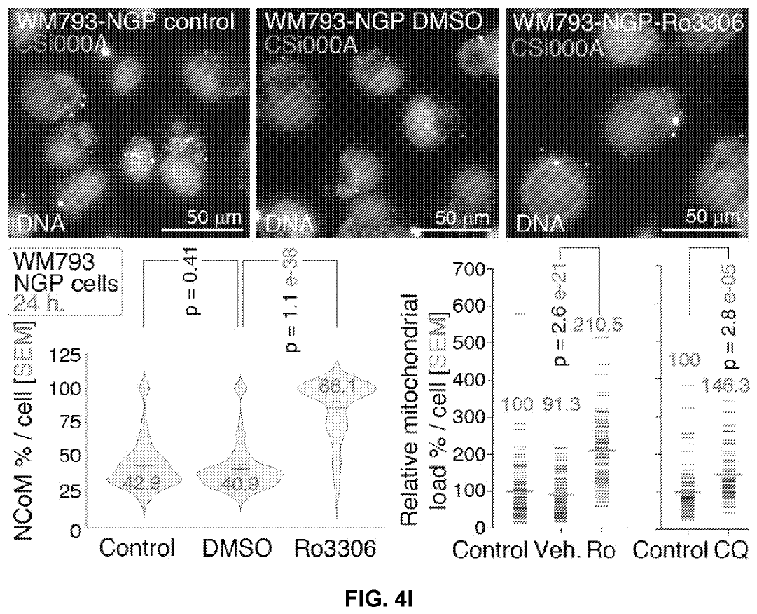

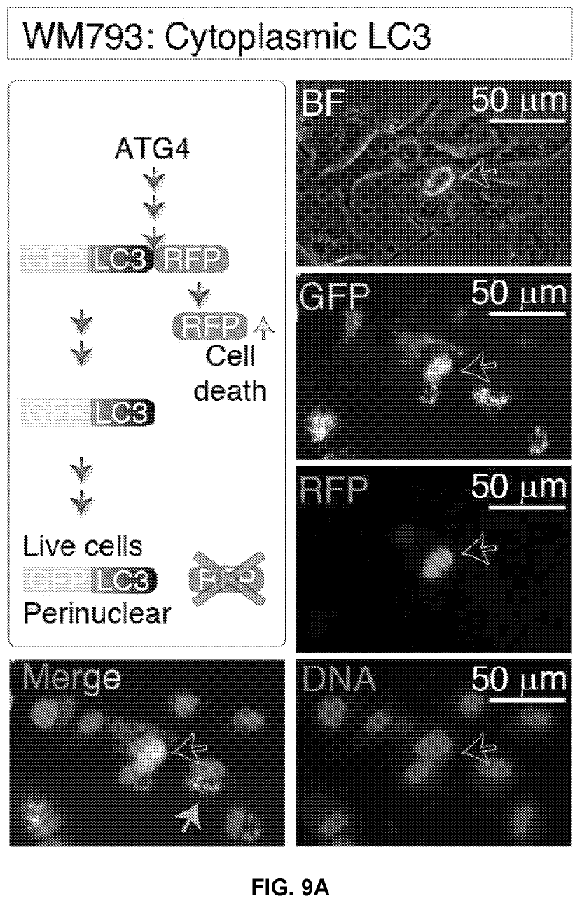

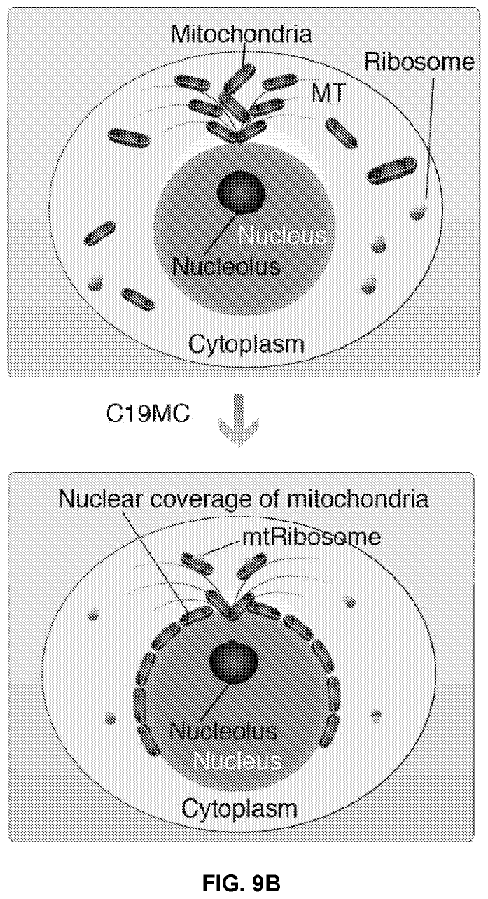

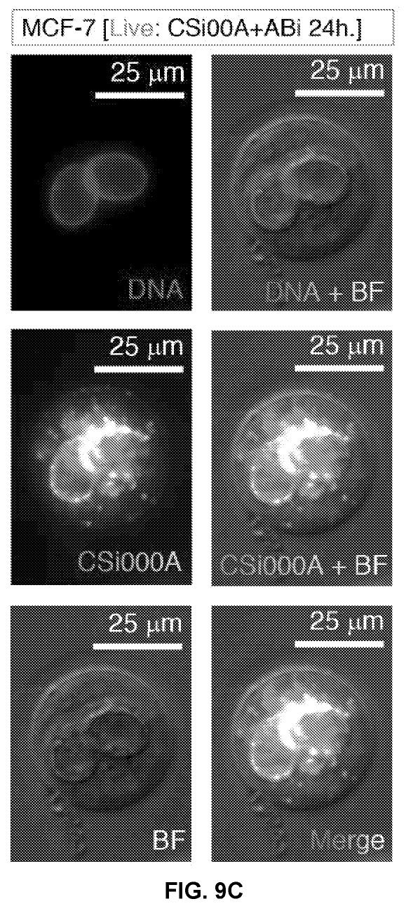

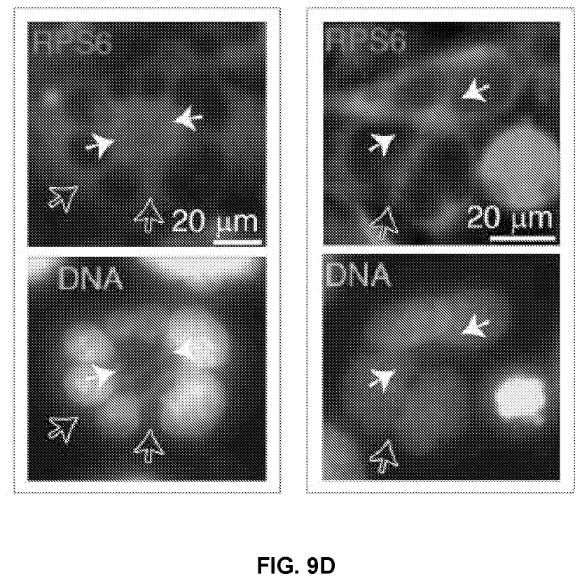

Autophagic flux was monitored within subcellular locations using pMRX-IP-GFP-LC3-RFP (Addgene: 84573, was obtained) construct (Kaizuka, T. et al. Mol Cell. 2016 64(4):835-849). Briefly, WM793 cells were transiently transfected with pMRX-IP-GFP-LC3-RFP plasmid followed by selection for 2 weeks. The cells were then plated at 50,000 cells/ml density for 24 hours in complete medium and then treated with 20 nM Aurora-B inhibitor (AZD1152-HQPA) in complete MEM for 24 hours. 30 minutes before the end of treatment duration, the cells were live stained with Hoechst-33342 before imaging using Zeiss Observer.Z1 microscope. Exposure times for GFP and RFP channels were kept constant. For WM793 cells, GFP fluorescence was detected in live cells and RFP was detected only in dying cells (RFP was constitutively degraded in live cells) therefore, GFP fluorescence was tracked to monitor autophagic flux within sub-cellular locations. As we compare GFP intensity within sub-cellular locations within single cells (FIG. 4C), the internal control RFP is not required. We refrain from comparing the autophagic flux between cells or between conditions because, the internal control RFP is constitutively degraded in WM793 cell system. Notably, RFP is degraded but not GFP in most of the cells therefore a cleavage between RFP and GFP-LC3 is evident, presumably by ATG4 of autophagy pathway (Kaizuka, T. et al. Mol Cell. 2016 64(4):835-849) (FIG. 9A).

### Starvation-Induced Autophagy, Autophagy Inhibition Experiments, and Data Visualization in R

WM793 cells that constitutively overexpress C19MC in a cluster-wide fashion (FIGS. 7D-7F) were plated in 6-well plates at a density of 50,000 cells/ml, 4 ml/well (200,000 cells/well). At 24 hours after plating, the cells were subjected to Aurora-B inhibition (20 nM for 24 hours) or vehicle DMSO, with or without starvation (fetal bovine serum and NEAA+L-glutamine amino-acid supplement withdrawal). Non-starved cells are considered fed, and labelled as fed cells (FIG. 4A). The cells were live stained with 10 nM Hoechst-33342 in existing media for 20 minutes, and imaged with constant exposure time settings. Using ImageJ software, nuclear area (excluding NiC area), Nuclear DNA intensity (Hoechst-33342 positive area intensity, excluding intensity from NiC area), and NiC area were measured for individual cells. The data were plotted in R using ggplot2 and ggridges packages (R version 4.1.3).
R code
#For DNA intensity
>ggplot(DFname, aes(x=‘DNA intensity’, y=Group, fill=stat(x)))+
xlim(0, 80)+
scale_y_discrete(expand=c(0, 0))+
   geom_density_ridges_gradient(scale=2, rel_min_height=0.005)+
   scale_fill_gradientn(colours=c(“yellow”, “red”, “black”),
      values=scales::rescale(c(0, 17.5, 35, 52.5, 70)))+
theme_bw( )
#Axis limits were based on the range of measurements.
#DF headers, axis limits, and rescale were changed in the above code for nuclear size and NiC size plots.

No visual cell death signs were observed at 24 hours of time point under starvation conditions but resulted in the suppression of DNA synthesis (FIG. 4A). This suppressed DNA intensity was considered the stage corresponding to G₁ stage of the cell cycle [the DNA intensity equivalent of 18×10,000 AU (ImageJ arbitrary units)](FIG. 4A). The dotted lines marking G₁ and G₂ phases were introduced in Adobe Photoshop CS5, and the percentage calculations based on dotted line values and t-test for significance were done using Microsoft Excel. Gates were set at 85 (for nuclear size) and at 200 (for NiC) that discriminates the Aurora-B inhibited conditions from starvation conditions in nuclear and NiC sizes respectively (FIG. 4A).

For autophagy inhibition experiments evaluating NiC (FIG. 4B), WM793 cells were plated in complete MEM (fed) for 24 hours, and were treated with or without 2 μM chloroquine (CQ) for 24 hours. NiC size (2D area) was measured as described above using ImageJ software. The data were plotted in R using ggplot2 and ggridges packages (R version 4.1.3).
R code
>ggplot(DFname, aes(x=‘NiCArea’, y=Group, fill=stat(x)))+xlim(−100, 600)+
scale_y_discrete(expand=c(0.5, 0.5))+
   geom_density_ridges_gradient(scale=1, rel_min_height=0.0001)+
   scale_fill_gradientn(colours=c(“yellow”, “red”, “black”),
      values=scales::rescale(c(0, 150, 300, 450, 600)))+
theme_bw( )
#Axis limits were based on the range of measurements.

A gate was set at 100, that discriminates the NiC size of Aurora-B inhibited conditions from chloroquine+Aurora-B inhibited conditions to show that the inhibition of autophagy by CQ increases the NiC size (FIG. 4B).

### Western Immunoblotting

Western immunoblotting was done as described previously (Jinesh, G. G. et al. Cell Death Discov. 2016 2:16003; Jinesh, G. G. & Kamat, A. M. Cell Death Discov. 2017 3:17023). Briefly, CRISPR-engineered cells in addition to parental control WM793 cells were plated at equal density of 50,000 cells/ml puromycin free complete MEM in 10 cm dishes (10 ml/dish). The cells were given a media change at 24 hour and after further 24 hours the cells lysed using whole cell lysis buffer, and clarified by centrifugation at 13,000 rpm for 10 minutes at 4 °C. The clarified lysates were quantified using Bradford assay with reference to bovine serum albumin (BSA). 20 μg lysates per lane were resolved in 10% SDS-polyacrylamide gels, transferred to nitrocellulose membranes overnight at 40V. The membranes were washed with PBS-Tween20 (PBST), blocked with 5% non-fat dry milk in PBST, and probed with SpCas9 antibody. The bands were visualized with Li-COR system using a secondary antibody compatible with Li-COR system. The membranes were then re-probed with REST/NRSF antibody (FIG. 3B).

### Tracking Proteasomal Degradation of SpCas9

Western blotting: To check whether SpCas9 is degraded by proteasomes or autophagy, WM793-NGP cells and WM793-CpG86 chopper CRISPR engineered cells were plated as described above in Western immunoblotting section. At 24 hours of plating the cells were treated with or without 1 μM MG-132 or 20 μM CQ for 24 hours. Remaining procedures were performed as described above with the modification that; an 8% SDS-PAGE gel was used to have a better resolution of HMW-spCas9 instead of 10% gel (FIG. 3C).

Immunofluorescence: To visualize the localization of SpCas9 in WM793-CpG86-CRISPR-chopper engineered cells, and in WM793-NGP cells, both cells were plated in 24-well pates at a density of 50,000 cells/ml/well. At 24 hours of plating, the cells were treated with 1 μM MG-132 for 24 hours and subjected to SpCas9-immunofluorescence as per the immunofluorescence and imaging protocol described above with Hoechst-33342 staining.

Quantification of SpCas9 localization: As we noted a perinuclear distribution of HMW-SpCas9 in MG-132 treated WM793-CpG86-chopper cells, which is different from MG-132 treated control NGP cells where SpCas9 show cytoplasmic localization, we counted the cells with perinuclear versus cytoplasmic SpCas9 distribution. We added more categories such as NiC localized (rare, as MG132 blocks NiC formation in most cells and Aurora-B inhibitor was not used), nuclear localized SpCas9 and so on (FIG. 8B).

### Cytogenetics: Structural Chromosomal Instability

Cytogenetic procedures were performed as described previously with minor modifications (Jinesh, G. G. & Kamat, A. M. Cancer Res. 2017 77(22):6144-6156). Briefly, cells were plated at a density of 100,000 cells/ml for 24 hours and treated with 20 ng/ml Colcemid for 16 hours in 10% FBS containing MEM. The mitotic cells and meiotic division-II/Ill cells that exhibit rounded off/poorly attached morphology were collected by mitotic shake-off method and pelleted at 1000 rpm. The pellets were resuspended with 7 mL of 0.067 mol/L KCl, allowed to swell for 2 minutes, fixed (two steps: 7 drops, and then 7 mL of fresh fixative 3:1 methanol: glacial acetic acid as described previously (Fletcher, J. Curr Protoc Hum Genet Chapter 10, Unit 10 13 (2001))). The cells in fixative were incubated at −20 °C overnight, washed with fresh fixative, subjected to chromosome spreading by high drop-in on “+ slides”, air-dried, aged for 1 hour at 70 °C using a slide warmer (Fisher Scientific), rehydrated and dehydrated using graded-series of ethanol, and stained with 2% modified Giemsa stain using CTG banding technique with modifications omitting trypsinization step (PBS, 70% ethanol, 95% ethanol, 3 dips each, air-dried, 2% Wright's modified Giemsa staining for 5 minutes, 3 distilled water dips and air-dried). Imaging was done using 40× objective and image processing was done as described in microscopy section. Structural alterations such as meiotic-bridged-chromosomes (MBCs) were considered as structural chromosomal instability.

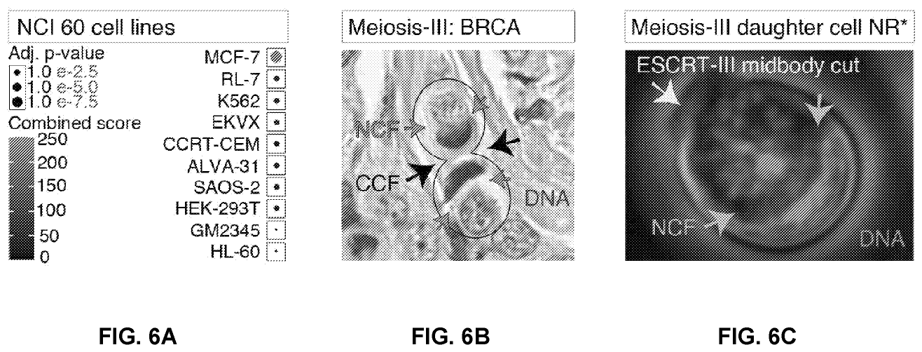

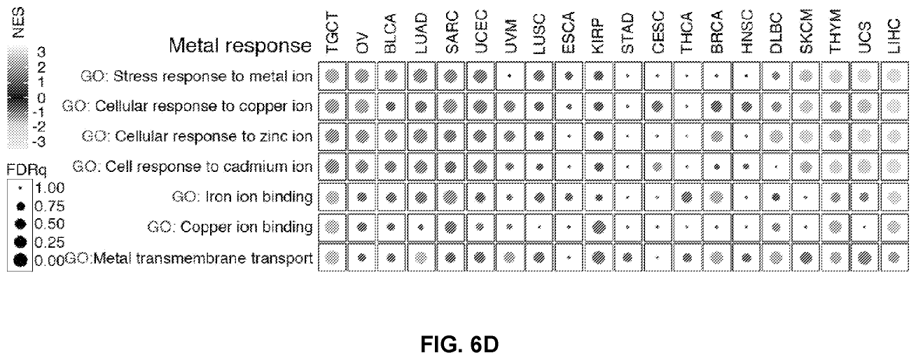

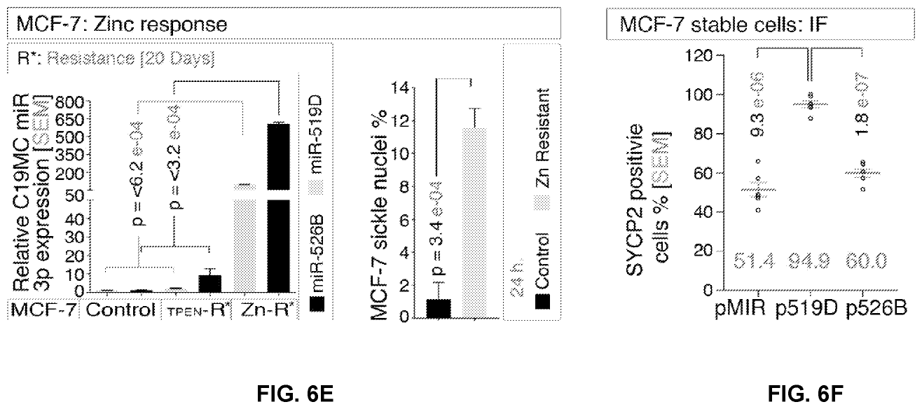

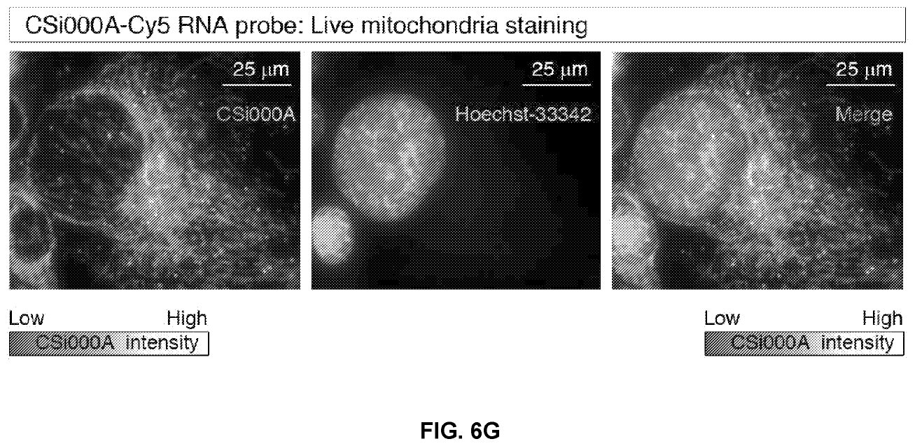

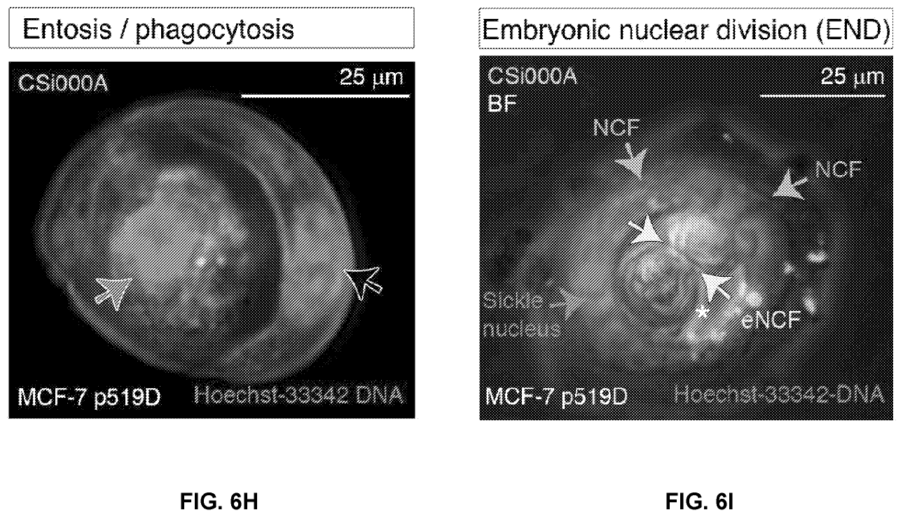

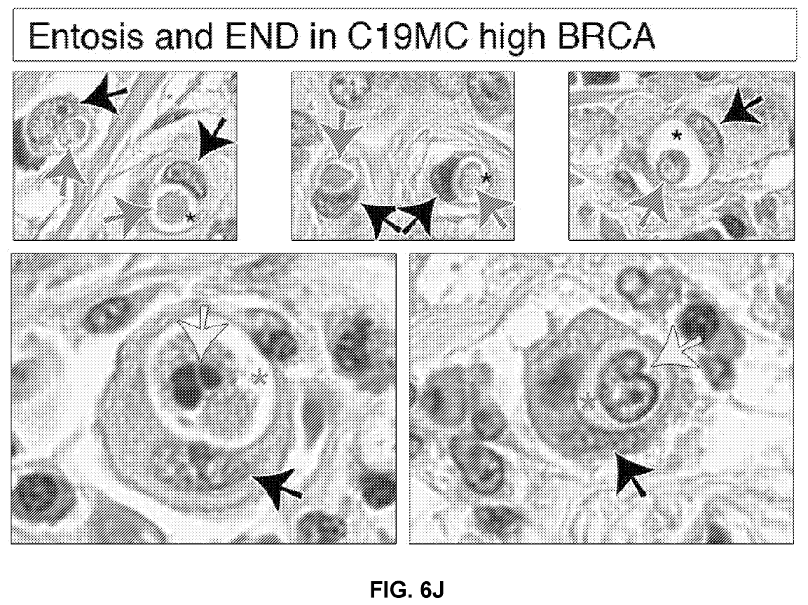

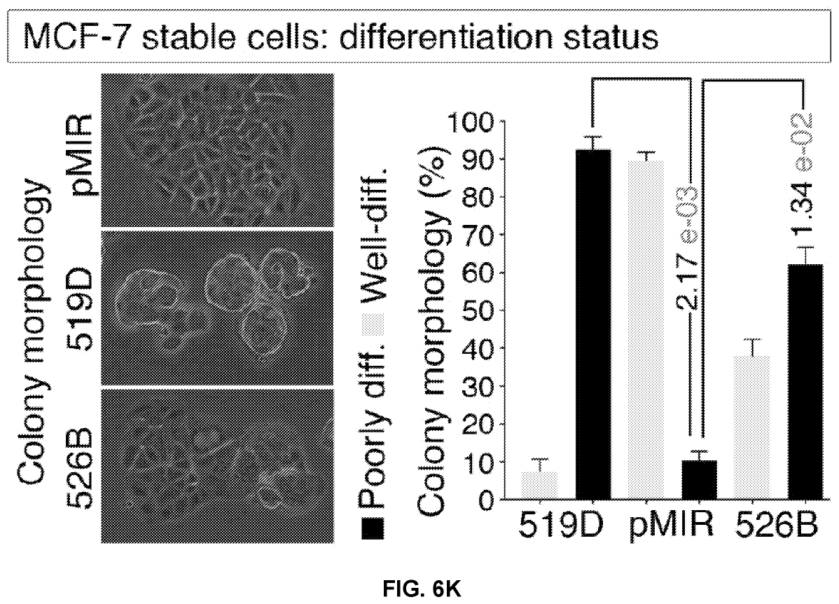

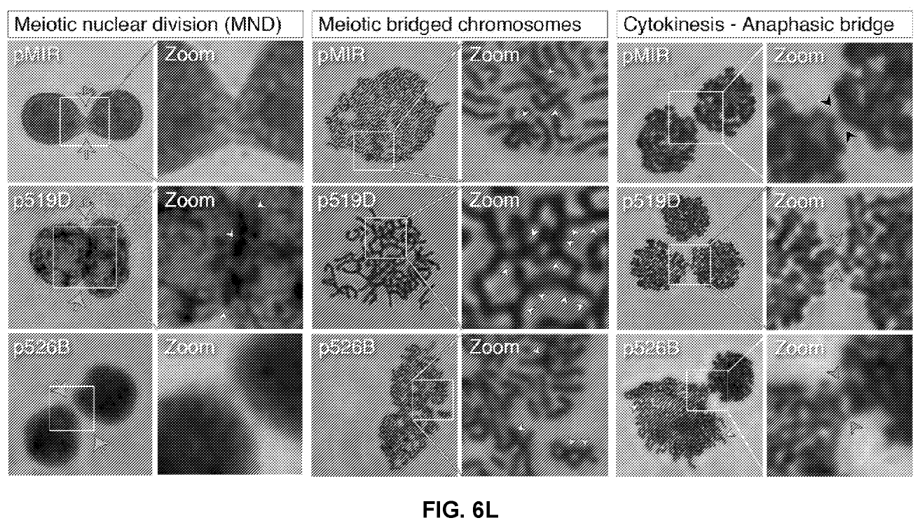

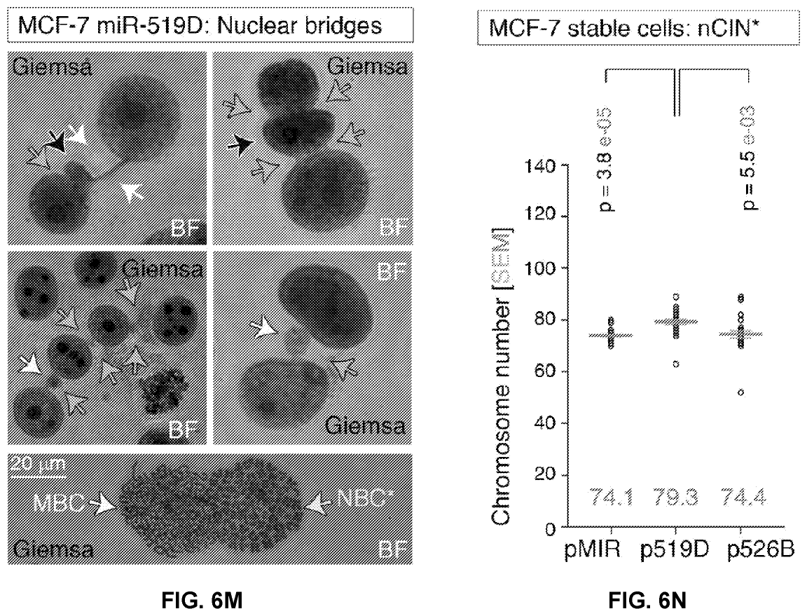

We observed numerous meiotic bridged chromosomes (MBCs) in miR-519D transfected stable cells which causes clumped chromosome tangles rather than spread chromosomes (despite adjacent cells were well spread, ruling out the possibility of variations in chromosome spread prep., as well as hypotonic swelling issues). The MBCs exhibit T-type bridges frequently than the X-type bridges to form the network using aberrant DNA-repair mechanism. X-type bridges could indicate the failure to resolve Holliday-junctions, or centromeric non-disjunction due to separase failures. Separase defects and non-disjunction in lagging chromosomes are frequently observed in C19MC miRNA overexpressed MCF-7 cells compared to MCF-7-pMir control cells (FIG. 6L).

Cytogenetics: Numerical Chromosomal Instability (nCIN)

As miR-519D overexpressed MCF-7 cells exhibited structural genomic instability (MBCs) which poses hurdles in measuring numerical chromosomal instability (please see previous section), we adapted counting individual chromosomes in cells that do not exhibit MBCs. Cytogenetic procedures were performed as described previously with minor modifications (Jinesh, G. G. & Kamat, A. M. Cancer Res. 2017 77(22):6144-6156) as described above in structural chromosomal instability section. 25 clean chromosomal spreads (without MBCs) were counted for chromosome numbers and were plotted as mean chromosomal number with standard error of mean using GraphPad Prism software (v7.04; La Jolla, CA, USA). Statistical significance was calculated using student's t-test in Microsoft excel. Numeric alterations compared to control were considered as numeric chromosomal instability (FIG. 6N). The experiments were performed at 2 months of maintenance after obtaining stable cells, to give an idea of timeline for the chromosomal gains in MCF-7-miR-519D cells.

### Meiotic Nuclear Division Imaging of Isolated Nuclei

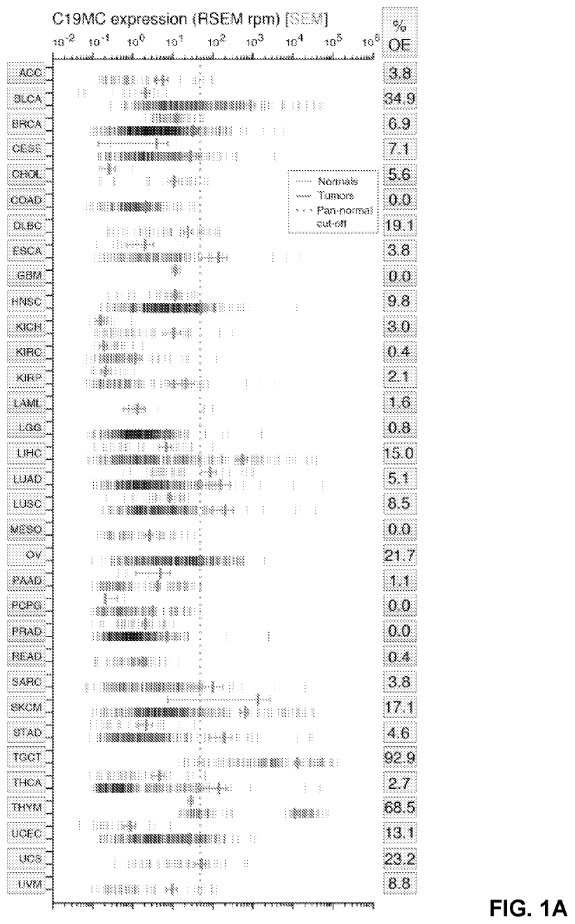

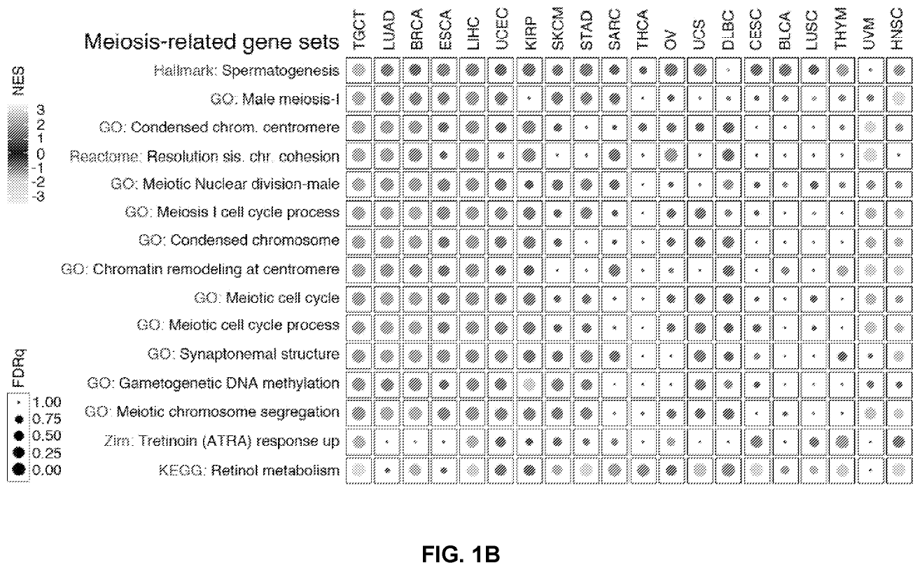

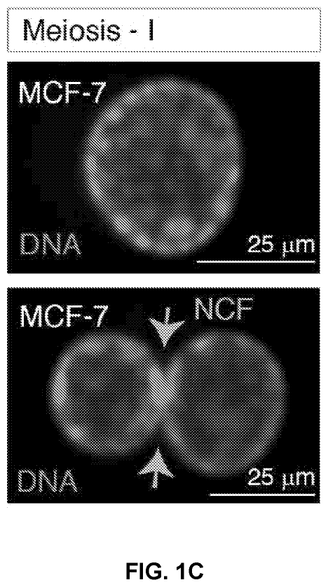

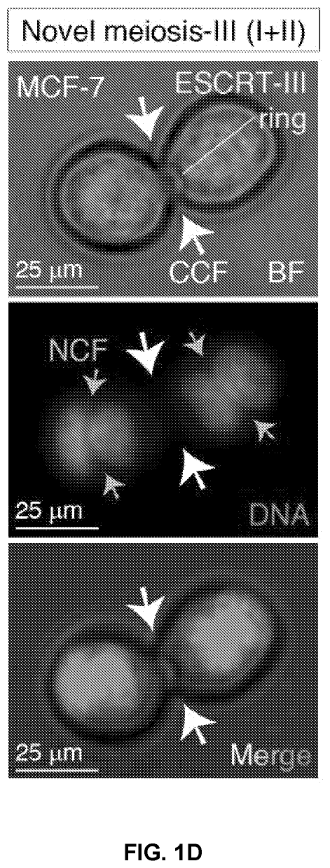

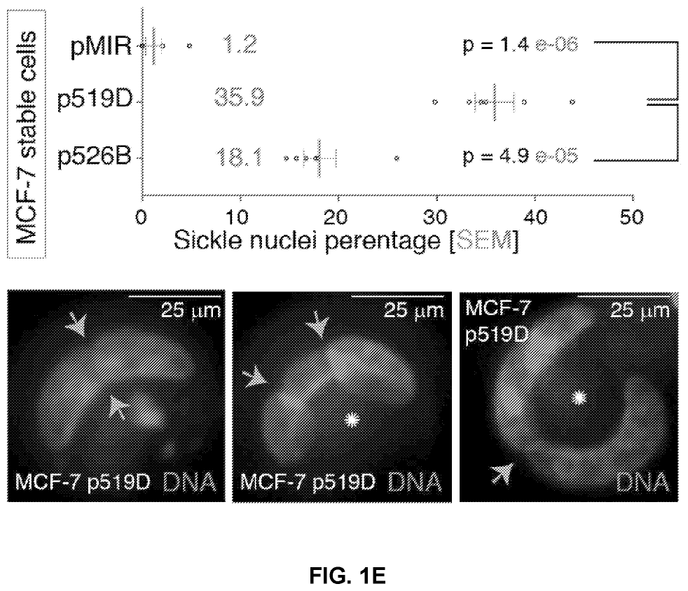

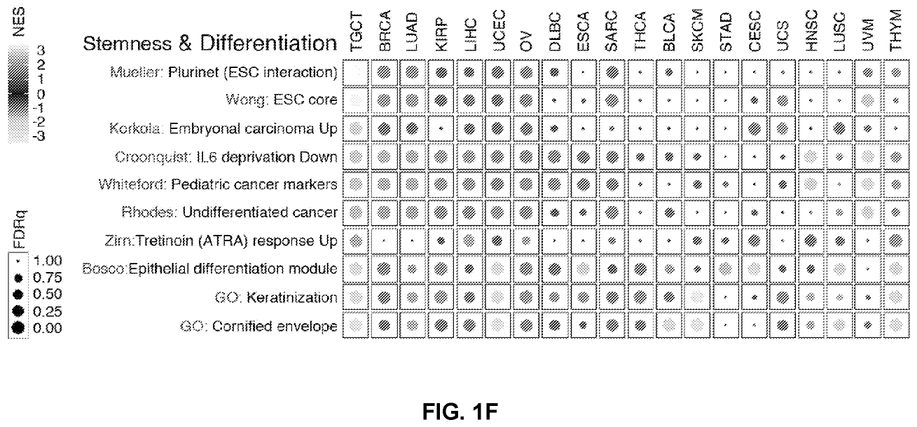

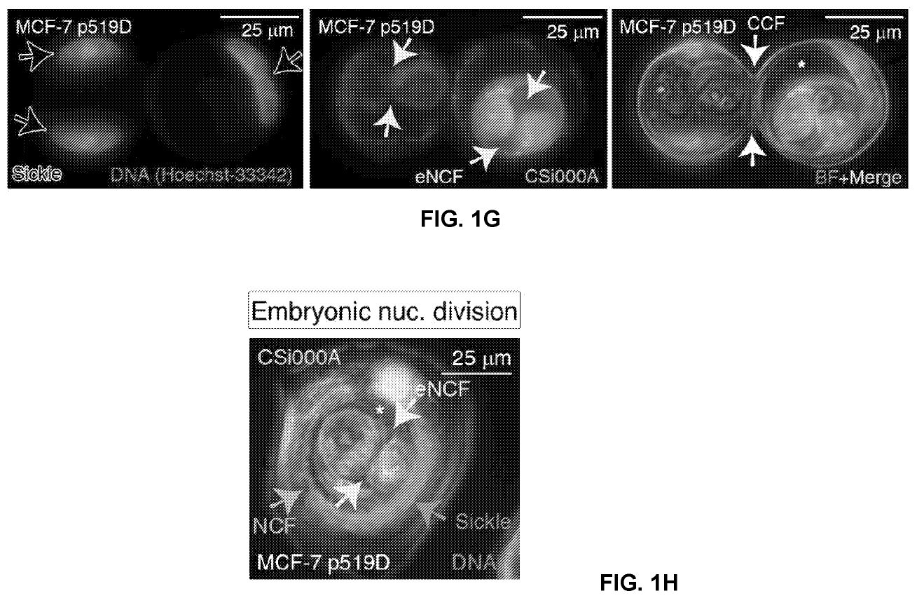

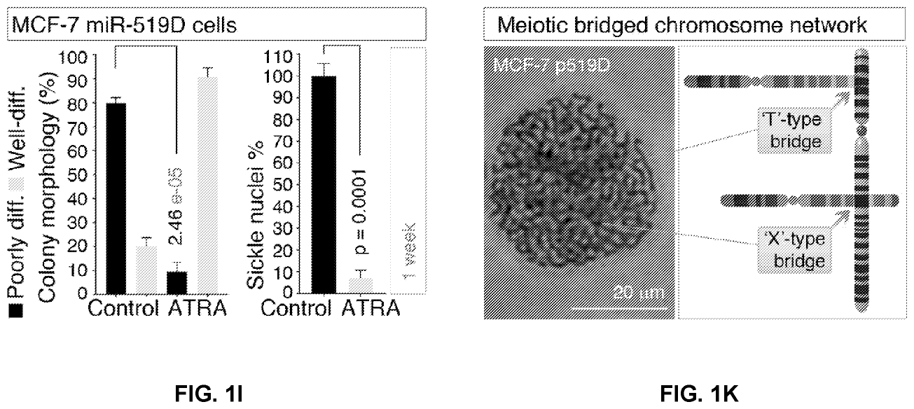

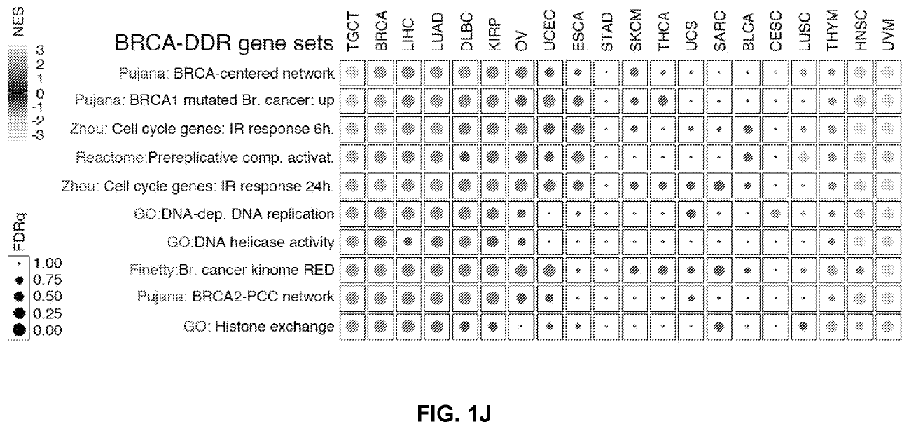

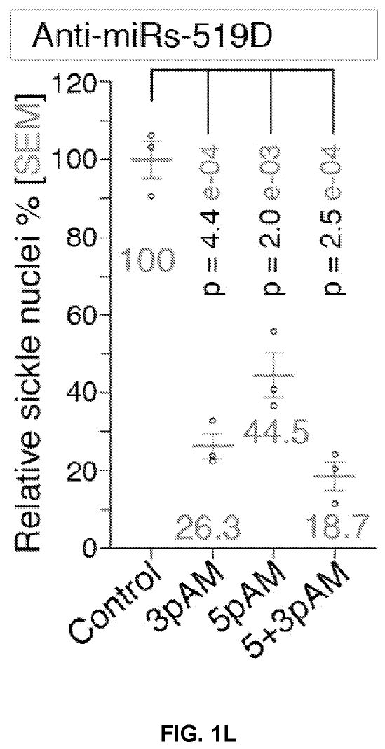

Cells were plated at a density of 100,000 cells/ml for 24 hours and treated with 20 ng/ml Colcemid for 16 hours in 10% FBS containing MEM to arrest the cells in chromatin condensed state. The cells exhibiting meiotic reduction division-I (FIG. 1C) with dumb-bell shaped nucleus exhibits rounded off morphology to a lesser-degree compared to mitotic cells, therefore these cells were collected by mitotic shake-off combined with extensive pipetting of the monolayer cells to dislodge relatively loosely attached cells undergoing meiotic nuclear division. The cells in media were then pelleted at 1000 rpm, pellets were resuspended with 7 mL of 0.067 mol/L KCl, allowed to swell for 1 minute (instead of 2 minutes time used for chromosome prep.), fixed [Step-1: 7 drops of fixative in hypotonic KCL solution and pelleted down at 1000 rpm, Step-2: removed the supernatant and resuspend the pellet in 7 mL of fresh (fixative 3:1 methanol: glacial acetic acid) and pelleted down at 1000 rpm]. The nuclei in fixative were incubated at −20 °C overnight, washed with fresh fixative, dropped onto “+ slides”, air-dried, aged for 1 hour at 70 °C, and stained with 2% modified Giemsa stain as described above. Imaging was done using 40× objective and image processing was done as described in microscopy section. Structural alterations in nuclei such as dumb-bell shaped, sickle-shaped nuclei (often lost the sickle shape to the most part by swelling), and MBC-derived mini-bridged nuclei were imaged (FIG. 6M).

### Nocodazole/Colchicine Arrest and Release of Meiosis-III Cells

To understand the nuclear status of daughter cells derived from Meiosis-III, we isolated Meiosis-III cells from MCF-7 cells by treating them with nocodazole or Colcemid for 16 hours followed by mitotic shake-off procedure described above. Of note, this will also include mitotic cells. The cells were washed twice in 37 °C PBS and plated in fresh complete MEM (Nocodazole/Colcemid release), and observed the cells at 30-minute intervals. At 2 hours post release, we found the appearance of daughter cells after ESCRT-cut of the mid-bodies to find that the daughter cells inherit two nuclei where one is sickle shaped with irregular chromatid condensation to give a hazy appearance with bright spots, whereas the other nuclei show well condensed individual chromosomes (FIG. 6C).

To understand the nuclear status of meiotic cells under inhibition of midbody abscission, we isolated meiotic cells from WM793 cells by treating them with nocodazole for 16 hours followed by mitotic shake-off procedure described above. Of note, this will also include mitotic cells. The cells were washed twice in 37 °C PBS and plated in fresh complete MEM with 20 nM Aurora-B inhibitor (Nocodazole release but inhibiting midbody abscission), and observed the cells at 2 hours post treatment. Bright field imaging was done to confirm the single cell status. Single cells arrested at MND stage with NiC were observed (FIG. 7J).

Well-Differentiated Vs. Poorly-Differentiated Colony Counts

MCF-7 cells stably transfected with C19MC miRNAs 519D, 526B or its control pMir, were plated at the density of 50,000 cells/ml in 6-well plates, with 4 ml puromycin free MEM/well. At 24 hours of plating, the cells were given a round of media change and incubated in 37 °C incubator for another 24 hours with 5% CO₂. The cells were then imaged at bright-field settings to count the colony morphology. A colony with both poorly-differentiated and well-differentiated mixture is counted as poorly-differentiated if more than 50% cells show poorly-differentiated morphology. Alternatively, if a colony shows more than 50% of its cells with well-differentiated morphology, then it is counted as well-differentiated colony. The counts were plotted in GraphPad Prism software with standard error of mean (FIG. 6K).

### Nuclear Counts in Living Cells

Stable pMIR, p519D or p526B transfected MCF-7 cells or WM793 cells were plated at a density of 50,000 cells/ml puromycin free MEM for 24 hours. At 24 hours, a round of media change were given and further incubated for 24 hours. The cells were then live stained with 10 nM Hoechst-33342 nuclear DNA stain for 20 minutes and were imaged for counting. Bright-field observation is made to ensure 4-daughter nuclei of Meiosis-III are not counted as 4 instead of 1 cell with sickle nuclei/MND (FIGS. 1E, 2A).

### Embryonic Nuclear Division (END) Visualization in Live Cells

MCF-7-pMir, MCF-7-miR-519D, and MCF-7-miR-526B stable cells were plated at a density of 50,000 cells/ml in 6-well plates for 24 hours in puromycin free complete MEM. The cells were transfected with 30 nM of Cy5-CSi000A using Lipofectamine-2000 reagent for overnight as described above. 24 hours after removal of transfection components, the cells were live stained with 10 nM of Hoechst-33342 for 20 minutes and imaged for Cy5 and Hoechst-33342 with bright field. The embryonic nuclear division (END) always located within potential cytoplasmic vacuoles (FIGS. 1G-1H, 6I) by engulfment of daughter cells through entosis (FIG. 6H). END was frequently observed in MCF-7-miR-519D stable cells. END is surrounded by sickle/sickle-nucleated cells both in vivo and in vitro (FIGS. 61-6J).

Metal Response Stimulation of Endogenous C19MC miRs

We previously found that, zinc chloride promotes C19MC miRNAs (Jinesh, G. G. et al. Sci Rep. 2021 11(1):12673). To examine the role of endogenous C19MC miRNAs in sickle nuclei formation, we generated zinc-resistant MCF-7 cells. Briefly, 200,000 MCF-7 cells/4 ml MEM were plated in 6-well plates for 24 hours and at 24-hour timepoint, treated with 1 mM ZnCl₂ for 24 hours to eliminate zinc sensitive cells. Remaining viable cells were maintained in 20 μM ZnCl₂ until the cells become confluent in about 20 days. The cells were plated at a density of 50,000 cells/well for 24 hours, and media was changed at 24 hours and incubated for further 24 hours. The cells were then live stained with 10 nM Hoechst-33342 and imaged for counting nuclear shapes.

Parallelly, zinc resistant cells were tested for C19MC miRNA expression after extracting RNAs, cDNA synthesis for miRs-519D, and 526B, and qRT-PCR.

Quantitative Real-Time-PCRs (qRT-PCRs) and Reverse Transcriptase PCRs (RT-PCRs)

qRT-PCRs: Quantitative real-time PCRs were performed as described previously (Jinesh, G. G. et al. Sci Rep. 2021 11(1):12673). RNAs were isolated using miRNeasy Mini Kit, quantified, and 250 ng RNAs were subjected to cDNA synthesis using Multiscribe reverse transcriptase, RNAse inhibitor, 10× buffer, dNTPs, (TaqMan MicroRNA Reverse Transcription Kit: ABI, Cat #4366596) and RT TaqMan Primers listed in Table 7. The cDNAs were subjected to qRT-PCR reactions using corresponding primers with probes and Taqman master mix listed in Table 7.

Comparative Ct (ΔΔCt) method was used to calculate the relative expression of miRNAs compared to pMir controls or untransfected controls as appropriate, after normalizing the values based on RNU6B. The results were plotted using GraphPad Prism software as bar graphs with standard error of mean as error bars (v7.04; La Jolla, CA, USA).

RT-PCRs: For RT-PCRs, Total RNA was isolated using TRIZOL reagent as per manufacturer's instructions and the reactions were performed as described previously (Jinesh, G. G. et al. Sci Rep. 2021 11(1):12673). Briefly, 20 μl cDNA synthesis reactions were performed using 1 μg RNA and High-Capacity cDNA Reverse Transcription Kit. 1.5M final concentration of betaine (from 5M stock) was added to the cDNA reactions. The thermocycler conditions used were, 25 °C for 10m, 37 °C for 120m and 85 °C for 5m. The cDNAs were diluted with 30 W of nuclease free water before subjecting 2.5 μl cDNA to PCR reactions. For the PCR step, 1M betaine (final conc.) was used along with regular PCR reaction components. A list of primers used in RT-PCRs are specified in Table 6. The thermocycler conditions for all PCR reactions have a denaturing (95 °C) time of 1-minute, annealing temperature of 60 °C (30 seconds) and 1 minute of extension time (72 °C), with 34 cycles. A final extension at 72 °C was given for 5 minutes. The PCR reactions were run with GeneRuler 100 bp DNA Ladder using 2% agarose gels. The gels were imaged using LI-COR Odyssey Fc imager (Lincoln, NE, USA).

### Nuclear Coverage of Mitochondria (NCoM) Quantification and Visualization in R

Mitochondria are known to have contact sites with various cytoplasmic organelles such as endoplasmic reticulum (ER) and nucleus. Mitochondria can have a wide-variety of distribution within cells such as perinuclear distribution. To understand the role of C19MC miRNAs in nuclear coverage of mitochondria (NCoM), we plated the CRISPR engineered WM793 cells (NGP and Choppers) in parallel to the parental cells at the density of 50,000 cells/ml in 6-well plates. At 24 hours, the cells were treated (not transfected) with 30 nM of Cy5-CSi000A RNA mitochondria probe in fresh MEM for further 24 hours. 30 minutes before the end of treatment duration, the cells were live stained with 10 nM Hoechst-33342 for imaging nucleus and mitochondria distribution using appropriate filters. Merged as well as individual channel images were exported and the 2D circumference coverage of nucleus (Hoechst-33342-positive DNA) by mitochondria were measured using ImageJ. Briefly, nuclear circumference (perimeter) was measured by drawing around the Hoechst-33342 positive DNA in addition to mitochondria signal (Cy5-CSi000A) overlapping perimeter of the nucleus. From these values the percentage of mitochondria covered nuclear perimeter was calculated for each cell. 100 such cells were measured for each cell type and the data were visualized in R using ggplot2 package as violin plots with standard error of mean (R version 4.1.3) (FIG. 4H). Please see below for R-codes. Statistical significances were calculated in Microsoft Excel using t-test option.

Tracking the role of autophagy and CDK1 in NCoM and mitochondrial load/cell: To examine the role of autophagy and CDK1 in regulating the nuclear coverage of mitochondria (NCoM), we used the WM793-CRISPR-NGP cells which have a predominant distribution of mitochondria at the perinuclear region. WM793-CRISPR-NGP cells were plated at a density of 50,000 cells/ml in 6-well plates (4 ml cells/well). At 24 hours after plating, the cells were treated with 2 μM chloroquine (CQ) or with 1 μM CDK1 inhibitor Ro3306 for 24 hours. DMSO was used as vehicle control and chloroquine does not have a vehicle control as it was freshly dissolved in distilled water. The cells were live stained for Hoechst-33342, imaged and NCoM measure as described above and the data were visualized in R using ggplot2 package as violin plots with standard error of mean (R version 4.1.3) (FIG. 4H-4I). Statistical significances were calculated in Microsoft Excel using t-test option.
R code
>ggplot(DFname, aes(x=Cells, y=NCoM))+
   geom_violin(trim=FALSE, fill=“grey90”, color=“black”)+
   stat_summary(fun.data=“mean_cl_normal”, geom=“errorbar”, width=0.15, linewidth=1, colour=“orange”)+
   stat_summary(fun=“mean”,
      geom=“crossbar”,
      width=0.2,
      colour=“red”)+
theme_classico
#Axis limits were based on auto package settings.

Tracking the relative mitochondrial abundance per cell: Relative mitochondria abundance per cell were calculated from the CSi000A signal/cell after normalizing the data to fix the control averages to 100% so that the variations between treatment conditions can be inferred in terms of percentage (FIG. 4I). The data were plotted in GraphPad Prism using aligned dot plot with standard error of mean and 75% transparency settings for data points. Statistical significances were calculated in Microsoft Excel using t-test option.

### TCGA H&E Diagnostic Slides/Histology

C19MC^{High} histology diagnostic slides were downloaded from GDC web portal by feeding in the TCGA deidentified IDs of C19MC^{High} patients. The slide images were examined using Aperio ImageScope software at maximum magnification. When pathologist's mark is available, the presence or absence of NiC and MND in cancer cells were examined within the mark. Sickle nuclei were restricted to few cancer types (please see main text) despite sickle-like nuclei with slender and curvy nuclei were observed in multiple C19MC^{High} cancers with teratoma histology (and were not considered sickle nuclei). Sickle nuclei with signet-ring carcinoma like morphology were also not considered as meiotic sickle nuclei, as in those signet-ring cells the sickle nuclei are potentially due to a large vacuole-like region that occupy the concave side of the curved nuclei and that could be mucin-filled bags or lipid drops that got lost during H&E preparation by organic solvents. Therefore, only sickle nuclei with condensed chromosomes on the concave side of the nuclei (FIG. 6B) or with embryonic nuclear division (END) (FIG. 6J) were considered meiotic sickle nuclei.

### Nuclear Margination of Chromatin and Mitochondria in Living Cells

MCF-7 cells were plated at 50,000 cells/ml in complete MEM for 24 hours and treated with Aurora-B inhibitor (AZD1152-HQPA) (20 nM) and Cy5-CSi000A (30 nM) for 24 hours and then the cells were subjected to 10 nM Hoechst-33342 nuclear DNA staining as described previously (Jinesh, G. G. & Kamat, A. M. Cancer Res. 2017 77(22):6144-6156; Jinesh, G. G. et al. Cell Death Differ. 2013 20(3):382-95). The cells with MND were imaged to show that the Hoechst-33342-positive DNA is marginated along the nuclear periphery (FIG. 9C) similar to MND in vivo in multiple C19MC^{High} cancer types). Notably, the mitochondria too aligned with the nuclear margin (FIG. 9C).

### Software, Packages, Web-Servers Used and Figure Compositing

A list of software used in this study are listed in Table 8. All graphs, plots, images exported from respective software were finally composited in Adobe Photoshop CS5 while keeping main plot area original but replacing axis fonts to have a uniform font size. Labeling, gate (dotted) lines were introduced in photoshop while corresponding data analysis with respect to the gated lines were done in original software or in Microsoft Excel. TCGA histology slides were opened in Aperio ImageScope software at maximum magnification and were transferred to photoshop cache or to Microsoft Paint, through Windows 10 screen shot snippet before being composited. Certain graphical schematic elements such as chromosomes (FIG. 7D & 7H) and nuclei with bridge sliced mitochondria (FIG. 9B) etc., were modelled in Lightwave-3D software and imported to Adobe Photoshop composite. Certain elements were added to the microscope image to mark the boundary of cells (FIGS. 2H, 6B). Arrows, arrow heads, scale bar lines of magnification of microscope images were also added using Photoshop in the composite, where the scale bars are based on original microscope image scale bars.

### Statistics

Statistical analyses (unpaired, nonparametric, Mann-Whitney test) for pan-cancer C19MC^{High} versus C19MC^{Low} groups were performed using GraphPad Prism software (v7.04; La Jolla, CA, USA). These analyses were also done between normal versus tumor samples but has a downside of lacking normal controls for multiple cancer types. The cumulative C19MC expression data were plotted as aligned dot plot with SEM and that data points with a transparency of 75%. For in vitro experiments, two-tailed, two-sample unequal variation-based t-test was performed to calculate statistical significance in Microsoft Excel 2010 or 2021 and the graphs were plotted either in excel or in GraphPad Prism software. All in vitro experiments were done with at least 3 replicates and in case of chromosome counts a minimum of 25 spreads instead of standard 10 spreads were counted. Experiments were repeated at different timelines to make sure reproducibility of key findings. For most individual cell measurements (for example, NiC size measurement), 300 cells were measured from replicates and were pooled to have comparisons with individual cell types or treatment conditions and to have the overall range of variability. More details on statistics and graph settings and codes using statistics within R packages such as ggplot2 were given in appropriate sections above. Results with borderline significances (p-value close to 0.05) were omitted or not chased further. Unless the non-significance is relevant to show that, the vehicles are not doing the phenotypic changes, the experiments with insignificant results were not included.

TABLE 1

| Human cell | Cancer | Commercial source & | | Fingerprinting/ |
|---|---|---|---|---|
| line | type | Cat.# | Maintenance medium | Mycoplasma status |
| MCF-7 | Breast | ATCC: Cat# HTB22 | MEM with 10% FBS + | STR-Confirmed/Negative |
| | | | Supplements* | |
| WM793 | Melanoma | ATCC: Cat# CRL-2806 | MEM with 10% FBS + | STR-Confirmed/Negative |
| | | | Supplements* | |
| IPC298 | Melanoma | DSMS: Cat# ACC 251 | MEM with 10% FBS + | STR-Confirmed/Negative |
| | | | Supplements* | |
| A375 | Melanoma | ATCC: Cat# CRL-1619 | MEM with 10% FBS + | STR-Confirmed/Negative |
| | | | Supplements* | |
| HepG2 | Liver | ATCC: Cat# HB-8065 | MEM with 10% FBS + | STR-Confirmed/Negative |
| | | | Supplements* | |
| Hep3B | Liver | ATCC: Cat# HB-8064 | MEM with 10% FBS + | STR-Confirmed/Negative |
| | | | Supplements* | |
| HEK293T | Kidney | ATCC: Cat# CRL-3216 | MEM with 10% FBS + | STR-Confirmed/Negative |
| | | | Supplements* | |
| SKHep1 | Liver | ATCC: Cat# HTB-52 | MEM with 10% FBS + | STR-Confirmed/Negative |
| | | | Supplements* | |
| HLF | Liver | JCRB: Cat# JCRB0405 | MEM with 10% FBS + | STR-Confirmed/Negative |
| | | | Supplements* | |
| SNU-182 | Liver | ATCC: Cat# CRL-2235 | RPMI with 10% FBS + | STR-Confirmed/Negative |
| | | | Supplements* | |
| T47D | Breast | ATCC: Cat# HTB-133 | MEM with 10% FBS + | STR-Confirmed/Negative |
| | | | Supplements* | |
| BT-459 | Breast | ATCC: Cat# HTB-122 | MEM with 10% FBS + | STR-Confirmed/Negative |
| | | | Supplements* | |
| MDA-MB-157 | Breast | ATCC: Cat# HTB-24 | MEM with 10% FBS + | STR-Confirmed/Negative |
| | | | Supplements* | |
| MDA-MB-231 | Breast | ATCC: Cat# HTB-26 | MEM with 10% FBS + | STR-Confirmed/Negative |
| | | | Supplements* | |
| MDA-MB-436 | Breast | ATCC: Cat# HTB-130 | MEM with 10% FBS + | STR-Confirmed/Negative |
| | | | Supplements* | |
| MDA-MB-468 | Breast | ATCC: Cat# HTB-132 | MEM with 10% FBS + | STR-Confirmed/Negative |
| | | | Supplements* | |
| SH-10-TC | Gastric | JCRB: Cat# RCB1940 | RPMI with 10% FBS + | STR-Confirmed/Negative |
| | | | Supplements* | |
| UMUC-14 | Bladder | Sigma Cat # 08090509 | MEM with 10% FBS + | STR-Confirmed/Negative |
| | | | Supplements* | |
| HT-1197 | Bladder | ATCC: Cat# CRL-1473 | MEM with 10% FBS + | STR-Confirmed/Negative |
| | | | Supplements* | |

*Supplements: NEAA, Penstrep, L-Glutamine, Sod. Pyruvate, & Vitamins

TABLE 2

| Reagent | Company | Catalogue # | Dose(s) used | |
|---|---|---|---|---|
| All-trans-retinoic acid (ATRA) | Cayman Chemicals | 11017 | 1 | μM |
| Hoechst-33342 | Cayman Chemicals | 15547 | 10 | nM |
| Aurora-B inhibitor AZD1152-HQPA | Cayman Chemicals | 11602 | 20 | nM |
| Actinomycin-D | Cayman Chemicals | 11421 | 1 ng/ml & 10 ng/ml* | |
| JC-1 mitochondria labeling agent | Cayman Chemicals | 15003 | 1 | μg/ml |
| CDK1 inhibitor Ro 3306 | Cayman Chemicals | 15149-5 | 10 | μM* |
| (S)-MG132 | Cayman Chemicals | 10012628-5 | 0.1 μM & 1 μM* | |
| Leptomycin-B (LMB) | Cayman Chemicals | 10004976-10 | 10 | ng/ml* |
| Chloroquine (CQ) | Cayman Chemicals | 14194 | 20 | μM |
| Cycloheximide (CHX) | Sigma | 01810 | 10 | μg/ml |
| Zinc chloride | Sigma | 208086 | 200 μM-1 mM | |
| Wright's modified Giemsa | Sigma | WG16 | 2% | |
| Nocodazole | Sigma | M1404 | 400 | ng/ml |
| Colcemid | Sigma | 15364 | 20 | ng/ml |
| DMSO | Sigma | D8418 | 1 μl/ml-2 μl/ml** | |
| 190 proof ethyl alcohol | Pharmco-Greenfield Global | 111000190 | 1 | μl/ml |
| Methanol | Fisher Scientific | A4524 | 1 | μl/ml |

| | | | |
|---|---|---|---|
| 5M betaine | Sigma | B0300-1VL | 1M final. |

*Dose selected to inhibit NiC formation;
**Serves as control if combination of two DMSO dissolved agents involved.

TABLE 3

| Reagent | Company | Catalogue # | Dose(s) used |
|---|---|---|---|
| Human chorionic gonadotrophin polypeptides A and B [hCG] | R&D Systems | 7727-CG- | 25 ng/ml |
| | | 010/CF | |
| Rabbit anti-SYCP2 antibody | Sigma | HPA062401 | 1:250 (IF*) |
| REST/NRSF antibody | Santacruz | sc-374611 | 1:100 (IF*); 1:500 |
| | Biotechnology | | (WB**) |
| Nucleolin/C23 antibody | Santacruz | sc-17826 | 1:100 (IF*) |
| | Biotechnology | | |
| RPS6 antibody | Santacruz | sc-74459 | 1:100 (IF*) |
| | Biotechnology | | |
| b-Tubulin antibody | Santacruz | Sc-166729 | 1:100 (IF*) |
| | Biotechnology | | |
| SpCas9 antibody [Clone: 14B6] | Genscript/ThermoFisher | A01936-40 | 1:250 (IF*); 1:500 |
| | | | (WB**) |
| Goat anti-Mouse IgG (H + L) Cross-Adsorbed Secondary | Life Technologies | A10524 | 1:1000 (IF*) |
| Antibody, Cyanine5 | | | |
| Goat anti-Rabbit IgG (H + L) Cross-Adsorbed Secondary | Life Technologies | A10523 | 1:1000 (IF*) |
| Antibody, Cyanine5 | | | |
| F(ab)2-Goat anti-Rabbit IgG (H + L) Cross-Adsorbed Secondary | Life Technologies | A-21430 | 1:1000 (IF*) |
| Antibody, Alexa Fluor 555 | | | |
| Lipofectamine 2000 | Life Technologies | 11668019 | MR*** |
| Lipofectamine RNAiMAX | Invitrogen | 13778075 | MR*** |
| Fast Digest Bbsl restriction enzyme | New England Biolabs | R3539S | MR* |
| LigaFast Rapid DNA Ligation System | Promega | M8221 | MR* |
| SOC media | G-Biosciences | 786-1280 | MR* |
| Plasmid MIDI-prep kit | Qiagen | 12143 | MR* |
| Plasmid Mini-prep kit | Qiagen | 27104 | MR*** |
| Illustra GFX PCR DNA and Gel Band Purification Kit | GE Healthcare | 28903470 | MR*** |
| Subcloning Efficiency DH5α Competent Cells | Life Technologies | 18265017 | MR*** |
| QIAamp DNA mini kit | Qiagen | 51304 | MR*** |
| pGEM-T Easy -TA cloning kit | Promega | A1360 | MR*** |
| Opti-MEM | Gibco | 31985070 | MR*** |
| Li-COR Goat anti-mouse secondary antibody-680LT | Li-COR | 925-88020 | 1:5000** |
| Li-COR Goat anti-rabbit secondary antibody-800CW | Li-COR | 926-32211 | 1:5000** |
| miRNeasy Mini Kit | Qiagen | 217004 | MR*** |
| TaqMan MicroRNA Reverse Transcription Kit | ThermoFisher/ABI | 4366596 | MR*** |
| Trizol | ThermoFisher Scientific | 15596026 | MR*** |
| GeneRuler 100 bp DNA Ladder | ThermoFisher Scientific | SM0243 | 2.5 μl/lane |

*IF, immunofluorescence;
**WB, Western blotting;
***MR, manufacturer's recommendation

TABLE 4

| Anti-miRNA | | Anti-miRNA/ | Target | Conc. |
|---|---|---|---|---|
| target | Sequence | probe-ID | region | used |
| miR-519D | 5′-CACUCUAAAGGGAGGCACUUUG-3′ | 519D3AM | 3p mature | 30 nM |
| | (SEQ ID NO: 15) | | | |
| miR-519D | 5′-AACAGAAAGCGCUUCCCUUUGGAGG-3′ (SEQ ID | 519D5AM | 5p mature | 30 nM |
| | NO: 16) | | | |
| miR-526B | 5′-GCCUCUAAAAGGAAGCACUUUC-3′ | 526B3AM | 3p mature | 30 nM |
| | (SEQ ID NO: 17) | | | |
| miR-526B | 5′-ACAGAAAGUGCUUCCCUCAAGAGG-3′ (SEQ ID | 526B5AM | 5p mature | 30 nM |
| | NO: 18) | | | |
| Non-targeting | 5′-Cy5-CGUGCGGGCCCGGGCGUGC-3′ | CSi000A | NT | 30 nM |
| | (SEQ ID NO: 19) | | | |
| Non-targeting | 5′-Cy5-CGUGCGGGGGGCCCCGUGC-3′ | CSi000B | NT | 30 nM |
| | (SEQ ID NO: 20) | | | |

TABLE 5

| | | | | Stable/transient* |
|---|---|---|---|---|
| Plasmids | Insert | Source/company | Cat. # | use |
| pMIR-CMV-GFP- | None | Vigene Biosciences | NA | Stable MCF-7 cells |
| Puro | | | | |
| pMIR-CMV-519D- | miR-519D | Vigene Biosciences | CR215546 | Stable MCF-7 cells |
| GFP-Puro | | | | |
| pMIR-CMV-526B- | miR-526B | Vigene Biosciences | CR215142 | Stable MCF-7 cells |
| GFP-Puro | | | | |
| PEGFP-C1-emerin | Emerin/EMD | Johns Hopkins | NA | Transient in WM793 |
| (EMD) | | University School | | cells |
| | | of Medicine | | |
| pMRX-IP-GFP- | GFP-LC3-RFP | Addgene | 84573 | Transient in WM793 |
| LC3-RFP | | | | cells |
| pSpCas9(BB)-2A- | No added Guide-sgRNA | Addgene | 62988 | Stable in WM793 |
| Puro (PX459) | cassette [NG] | | | pooled cells [NGP]** |
| V2.0 | | | | |
| pSpCas9(BB)-2A- | Chopper-1- | Cloned (See below) | NA | Stable in WM793 |
| Puro (PX459) | GGGCACGGTGGTTCCCATCA- | | | pooled cells |
| V2.0-Chopper-1 | sgRNA cassette (SEQ ID | | | [Choppers-P]** |
| | NO: 21) | | | |
| pSpCas9(BB)-2A- | Chopper-2- | Cloned (See below) | NA | Stable in WM793 |
| Puro (PX459) | AGAGGGGTCCAGGCTGGGCA- | | | pooled cells |
| V2.0-Chopper-2 | sgRNA cassette (SEQ ID | | | [Choppers-P]** |
| | NO: 22) | | | |

*, ** See below for cloning and transfection procedures; **Chopper-1 and 2 were transfected together

TABLE 6

| Primer | | Annealing | Product | |
|---|---|---|---|---|
| IDs | Sequence | temp. | size | Template |
| Colony | 5′-TAAAATGGACTATCATATGCTTACCG-3′ | 60 °C | 188 bp | Bacterial colony |
| PCR-F | (SEQ ID NO: 23) | | | (plasmid: |
| Colony | 5′-AACAAAAAAGCACCGACTCGGTGCC-3′ | 60 °C | | pSpCas9[BB]-2A- |
| PCR-R | (SEQ ID NO: 24) | | | Puro [PX459] V2.0) |
| Chop86-F | 5′-TTTCGCCCGCTGCCTGGAAGTATC-3′ | 60 °C | 999 bp if not | Human genomic |
| | (SEQ ID NO: 25) | | chopped; | DNA |
| Chop86-R | 5′-CCGATTCTCCCACCTCAGCCTCCC-3′ | 60 °C | Lower size | |
| | (SEQ ID NO: 26) | | ladder(s) if | |
| | | | chopped off* | |
| SpCas9-F | 5′-TTCTGGCGGTTCTCTTCAGCCGGG-3′ | 60 °C | 468 bp | Human genomic |
| | (SEQ ID NO: 27) | | | DNA-CRISPR cells. |
| SpCas9-R | 5′-CGGGACGGCCCTTCTCCTCCGGG-3′ | 60 °C | | |
| | (SEQ ID NO: 28) | | | |
| IGFBP3-F | 5′-GGGCGCGACCCACGCTCTGGGCC-3′ | 60 °C | 350 bp | Human genomic |
| | (SEQ ID NO: 29) | | | DNA. |
| IGFBP3-R | 5′-GGCACTAGCGTTGACGCAGAGCCC-3′ | 60 °C | | |
| | (SEQ ID NO: 30) | | | |

*The ladders <999 bp is formed due to variability in 13-repeat knock-out in pooled cells. A single band can be obtained from single cell clones.

TABLE 7

| miRNA target/ | Assay | Catalogue | | |
|---|---|---|---|---|
| master mix | IDs | number | Company | Step in which used |
| RNU6B Control | 001093 | 4427975 | ThermoFisher | cDNA synthesis |
| hsa-miR-519d-3p | 002403 | 4427975 | ThermoFisher | cDNA synthesis |
| hsa-miR-520g-3p | 001121 | 4427975 | ThermoFisher | CDNA synthesis |
| hsa-miR-526b-3p | 002383 | 4427975 | ThermoFisher | cDNA synthesis |
| TaqMan Universal PCR | NA | 4324018 | Life | PCR step with corresponding |
| Master Mix | | | Technologies | primer FAM solutions (pink) |

TABLE 8

| Software/Web server | Package | Version | Company |
|---|---|---|---|
| GraphPad Prism | NA | 7.04 | GraphPad Prism |
| Photoshop | NA | CS5 | Adobe |
| Aperio ImageScope | NA | 12.4.6.5003 | Leica Biosystems |
| ImageJ | NA | 1.53k | NIH, USA |
| SnapGene viewer | NA | 6.0.6 | SnapGene |
| EndNote | NA | X9.3.3 | Clarivate |
| IGV | NA | 2.12.2 | Broad Institute, USA |
| FinchTV | NA | 1.4.0 | Geospiza |
| Lightwave-3D | NA | 10 | NewTek |
| R studio | NA | 2021 Sep. 2 Build 382; | Posit |
| | | “Ghost Orchid” Release | |
| | | for Windows | |
| R studio | ggplot2 | Built under R version 4.1.3 | NA |
| R studio | ggRidges | Built under R version 4.1.3 | NA |
| Microsoft Office | Excel | 2021 | Microsoft |
| Microsoft Office | Word | 2021 | Microsoft |
| GeneVenn | NA | Online server | GeneVenn |
| EnrichR | NA | Online server | EnrichR |

### Results

### Meiosis-III in C19MC^{High} Cancers

C19MC is overexpressed in subsets of tumors from multiple cancer types with the maximum in testicular germ-cell tumors (TGCT), and we focused on 20 cancer types that allow sufficient C19MC^{High} and C19MC^{Low} group stratification (FIG. 1A). GSEA analysis revealed a strong upregulation of a coherent cluster of meiosis-related gene-signatures enriched in C19MC^{High} tumors (FIG. 1B). While TGCT has the highest enrichment with spermatogenesis and meiosis gene-signatures as one can expect based on tissue type, multiple other C19MC^{High} cancers also exhibited these gene-signature enrichments (FIG. 1B). We first asked if meiosis can happen in non-germline cells. Interrogation of meiosis gene signature in EnrichR pointed to the MCF-7 breast cancer cells (FIG. 6A). We found rare meiotic reduction division figures in MCF-7 cells where, the condensed chromosomes appeared to anchor at the nuclear lamina from within, while the nucleus undergoing division with a dumb-bell shape created by nuclear cleavage-furrow (NCF) (FIG. 1C). In addition, we found a novel form of meiosis, and call it as “meiosis-III” where, the reduction nuclear division and cytokinesis progress simultaneously (in contrast to normal meiosis where it is supposed to be sequential: meiotic reduction division-I, and then chromosome duplication and a mitosis-like division-II) (FIG. 1D). Notably, meiosis-II does not display discrete chromosome condensation but displays diffuse-type irregular-condensation. Overexpression of individual C19MC miRNAs in MCF-7 cells dented the meiotic nuclear shape especially by miR-519D to a sickle shape with multi-plane NCFs in it. Notably, a nucleolus-like compartment seemed to have enlargement to create sickle cells upon opening-up on one side (FIG. 1E). Such sickle nucleated meiosis-II can happen in C19MC^{High} human breast cancer and the cytokinesis ESCRT-cut results in the binucleated daughter cells with one sickle nucleus and other nucleus with or without chromosome condensation (FIG. 6B-6C). Stimulation of endogenous C19MC miRNAs by Zinc-metal response also promotes sickle nuclei (FIG. 6D-6E). Notably the meiotic-synaptonemal complex protein SYCP2 was promoted by miR-519D than 526B (FIG. 6F) suggesting 519D has a prominent role in meiosis.

### Undifferentiated ESCs vs Differentiation

Among all GSEA enrichments in C19MC^{High} tumors, TGCT exhibited the embryonic stem cell (ESC) signature as the top candidate which is accompanied by undifferentiated, IL-6-inflammatory, embryonal carcinoma, and pediatric cancer marker signatures in multiple cancer types (FIG. 1F). Using the mitochondria labeling-RNA-probe Cy5-CSi000A (FIG. 6G) we detected embryonic nuclear division (END) with embryonic NCF (eNCF) in meiosis-III in MCF-7-miR-519D overexpressing cells (FIG. 1G-1H). This nuclear DNA labeling by Cy5-CSi000A is potentially due to entosis and subsequent mixture of mitochondria and nuclear genomes, as the END is always observed within vacuolar structures of MCF-7-miR-519D cells and in C19MC^{High} breast cancer (FIG. 1G-1H, 6H-6J). Furthermore, 80-90% of MCF-7-miR-519D cells exhibit undifferentiated morphology which can be reversed to differentiated state and sickle nuclei-depleted state upon all-trans retinoic acid (ATRA, a meiotic promoter in male germ cells (Pellegrini, M. et al. Cell Cycle. 2008 7(24):3878-88)) induction (FIG. 1I, 6K) suggesting that, miR-519D delayed the MND and that, the meiotic promoter ATRA overcomes the delay.

### Meiotic-Bridged-Mini-Nuclei & CIN

GSEA results in C19MC^{High} tumors also displayed enrichments of BRCA-centered DNA-damage response gene-sets (FIG. 1J). The NCF in MCF-7-519D sickle nucleated cells usually exhibited tangled DNA condensate (FIG. 1E). Isolation of meiotic nuclei and cytogenetics revealed that, meiotic-bridged chromosome (MBC) network (FIGS. 1K, 6L), and chromosome-cohesion resolution defects such as anaphase-bridge (FIG. 6L) interfere with MND to potentially activate the “NoCut checkpoint” (Amaral, N. et al. Nat Cell Biol. 2016 18(5):516-26; Carlton, J. G., et al. Science. 2012 336(6078):220-5). In support of this, we observed meiosis-III nuclei in a bridged fashion where the MBCs accumulate as bridged mini-nuclei in between the large “supposed-to-be” daughter nuclei (FIG. 6M). Of note, meiotic-bridged mini-nuclei are different from micronuclei, as micronuclei do not have bridges to main nuclei. Interestingly, MBCs and non-MBCs segregate during MND (FIG. 6M). While MBCs demonstrate structural chromosomal instability (CIN) and makes it difficult to count chromosome numbers, we evaluated numerical chromosomal instability in cells that lack MBCs. Following 2 months of maintenance, the MCF-7-519D cells gained approximately 5 chromosomes on average (FIG. 6N). Therefore, C19MC utilizes DNA-damage response to generate MBCs to hamper the progression of MND through activation of NoCut checkpoint and generating multinucleated cells with structural and numerical chromosomal instability (sCIN and nCIN).

### Nucle(ol)ar Invasion of Cytoplasm (NiC)

GSEA results of 20-different C19MC^{High} cancer types revealed a strong upregulation of influenza and HIV infection gene-signatures in C19MC^{High} TGCTs, in addition to other viral-inducible gene-signatures across various cancer types. (FIG. 7A). The influenza and HIV infection signatures comprises genes regulating, rDNA-transcription, rDNA-translation, proteasome, nucleoporins, nuclear import/export: FIG. 7B) where the rDNA-influenza signature (RPL and RPS genes) is upregulated in 1020 CCLE cell lines (FIG. 7B). A search for influenza signature pointed to WM793 melanoma cell line which has high cluster-wide C19MC expression based on CCLE-NGS data and our qRT-PCR verification of C19MC hyper-expression (FIG. 7C-7F). Having baseline hyper-C19MC expression, WM793 exhibits sickle nuclei and MND (FIG. 2A), nuclear membrane margination of condensed chromosomes and MBCs at NCF (FIG. 7G). TCGA GSEA data also revealed a lack of ESCRT (Carlton, J. G., et al. Science. 2012 336(6078):220-5) enrichment but had anaphase-promoting complex (APC) and Aurora-B-related gene-signature enrichments (FIGS. 2B, 7H) suggesting a major role for Aurora-B and NoCut checkpoint in multinucleation.

Inhibition of Aurora-B resulted in the formation of “Hoechst-33342 stained-DNA-negative” sub-domains within nuclei in 58% cells, which we call it as nucle(ol)ar-invasive cytoplasmic compartment (NiC) based on the results of following experiments in this study (FIG. 2c). This compartment is formed by the invagination/self-invasion of nuclear membrane as visualized by GFP-emerin (marks the inner nuclear membrane (Lee, K. K. et al. J Cell Sci. 2001 114(Pt 24):4567-73; Ungricht, R. & Kutay, U. Nat Rev Mol Cell Biol. 2017 18(4):229-245)) (FIG. 2D), and NiC encloses mitochondria as visualized with JC1 staining and independently with a RNA probe, Cy5-CSi000A (FIG. 2E-2F). Cells generally formed a single NiC per nucleus but frequently double NiCs are formed in cells undergoing MND that are also exposed to Aurora-B inhibitor with or without nocodazole arrest and release (FIGS. 7I-7J). NiC is a closed compartment and it encloses “Giemsa-positive DNA” despite it lacks “Hoechst-33342-negative DNA” as shown in hypotonic-salt-isolated nuclei (FIG. 7K). Giemsa-positive DNA raised the possibility that it could be nucleolus that was engulfed by the invading nuclear membrane. We found the accumulation of nucleolin (nucleolus marker), RPS6 (Ribosomal constituent regulating spermatogenesis (Wang, L. et al. Semin Cell Dev Biol. 2022 121:53-62)) and REST (Neuronal restricting transcription factor) within the NiC (FIG. 7L). Therefore, we conclude that, NiC is a nuclear membrane-guarded nucle(ol)ar-cytoplasmic hybrid-compartment (nucleoli are not usually bound by membranes (Hirose, T., et al. Nat Rev Mol Cell Biol. 2023 24(4):288-304)) that encloses cytoplasmic materials such as mitochondria, proteins and possibly ribosomes (Ribosomes are usually assembled at nucleoli (Hirose, T., et al. Nat Rev Mol Cell Biol. 2023 24(4):288-304; Hori, Y., et al. Nat Rev Mol Cell Biol. 2023 24(6):414-429) and can enter into NiC as mitochondrial ribosomes).

### Meiotic Regulators Determine NiC Size

Considering the novelty of NiC in the context of IAV+HIV signatures, and multiple viruses [such as influenza-A virus (IAV), human immunodeficiency virus (HIV-1), cytomegalovirus (CMV), severe acute respiratory syndrome corona virus (SARS-CoV)] localizes to a large nuclear sub-domain positive for nucleolar markers (Hiscox, J. A. Arch Virol. 2002 147(6):1077-89), we examined this compartment to understand how it is regulated and how it is related to meiosis. We identified that, the pregnancy hormone human chorionic gonadotrophin-A and B (hCG) RNAs are consistently promoted in C19MC^{High} tumors (FIG. 7M) and hCG is also a known meiotic-promoter (Tiwari, M. & Chaube, S. K. Dev Growth Differ. 2017 59(7):603-614). Interestingly, hCG alone or in combination with Aurora-B inhibitor increased the size of NiC and raised the frequency of multinucleated cells (FIG. 2G-2H). Combination of chemical promoter of meiosis ATRA (Li, H. & Clagett-Dame, M. Biol Reprod. 2009 81(5):996-1001), with hCG further enlarged the NiC compartment size with Aurora-B inhibitor (FIG. 21). Therefore, NiC is relevant to multinucleation, and is regulated by meiotic promoters.

Next, we examined how NiC is related to C19MC and the IAV+HIV signatures (which constitutes the rDNA-ribosome biogenesis and homeostasis pathway (Hori, Y., et al. Nat Rev Mol Cell Biol. 2023 24(6):414-429)). Inhibition of any arm of IAV+HIV/rDNA signature or CDK1 (which regulates rDNA transcription through MYCN stability (van Riggelen, J., et al. Nat Rev Cancer. 2010 10(4):301-9) and translation of 5′-TOP ribosomal RNAs (Haneke, K. et al. J Cell Biol. 2020 219(3):e201906147)) blocks the formation of NiC (FIG. 2J, 7N-7O). Likewise, interfering C19MC with anti-miRs impeded NiC and MND (FIG. 2K, 7P). Therefore, C19MC regulates the NiC and MND through rDNA pathway.

### RPS6, REST, & Nucleolin are NiC-Cargoes

Although Giemsa positive DNA is localized within NiC, we anticipated a cargo traffic in and out of NiC would determine its size. We found that, ribosomal small subunit protein RPS6 localizes and accumulates approximately 200% more at NiC when Aurora-B is inhibited, and this process was blocked by rDNA pathway inhibitors or by CDK-1 inhibitor (FIG. 2L). Similarly, nucleolin and REST/NRSF cargos were also trafficked in and out of NiC (FIG. 2M). Therefore, CDK1 regulated the cargo-influx and Aurora-B regulates the cargo-efflux at NiC and that, a C19MC-dependent IAV+HIV infection signature drives cargo-flux at NiC. Notably, the data from chemical inhibitors are robust compared to the anti-C19MC-miR data. Therefore, we aimed for a genetic C19MC-targeting approach to further validate these data.

### CpG86 Regulates C19MC and Proteasome

To understand the C19MC expression, we sought to CRISPR-engineer the genome at C19MC landscape. There is a potential transcription start at the C19MC upstream CpG86-island. CpG86 has 13 specific-repeats (FIG. 3A) and exhibits hypervariability in this repeat region across 19 cell lines examined with a lowest repeat number in MCF7 cells (missing 8-repeats naturally) (FIG. 8A). We generated two CRISPR-CpG86-chopper guide RNA-constructs (using the SpCas9 system (Ran, F. A. et al. Nat Protoc. 2013 8(11):2281-2308)) which collectively target 16-sites within the CpG86 13-repeat-region (FIG. 3A). We CRISPR-knocked-out 10 of these repeats in WM793 cells (FIG. 3A, 8A-8B). Interestingly, the spCas9 control construct (NGP) suppressed C19MC miRNA expression by approximately 95% whereas, the CRISPR-CpG86-choppers restored the C19MC expression (FIG. 3A). A deeper examination revealed that, both control and chopper cells had SpCas9 in the genome and expressed it at the transcript level. However, SpCas9-protein is expressed only in control, but was degraded by proteasome in CRISPR-CpG86-chopper-transfected cells (FIG. 3B-3C). Inhibiting proteasome but not autophagy results in the accumulation of potentially ubiquitinated SpCas9-protein of >500 kDa at perinuclear region (FIG. 3C). Of note, SpCas9 is known for multi-site ubiquitination to generate similar higher molecular weight (HMW) band (ErgOnay, T. et al. Life Sci Alliance. 2022 5(4):e202101078). Therefore, the CpG86-repeats act as the genomic switch for C19MC repression by SpCas9, and C19MC-directed ubiquitin-proteasome activation degrades SpCas9 to overcome the repression at CpG86. Consistently, Proteasome gene-signatures are enriched in multiple TCGA cancer types and that, SpCas9 in chopper cells accumulate at perinuclear region predominantly, and at NiC to a lesser extent when proteasomes were inhibited (FIG. 8B). We evaluated the meiotic-synaptonemal protein in these CRISPR-engineered cells and found that, SpCas9 indeed suppress SYCP2 but choppers restored it (FIG. 3D).

### C19MC Drives NiC and Cargo Traffic

To understand the cause-effect relationship between C19MC and cargo traffic in and out of NiC, we utilized the CRISPR-engineered cells. Of note, these are pooled CRISPR cells and hence clonal effects on phenotype deviations are negligible. SpCas9 in NGP cells (which suppresses C19MC miRNA expression) blocked the formation of NiC by >90%, and NiC influx of RPS6 and nucleolin to a similar extent upon Aurora-B inhibition (FIG. 3E-3G). Conversely, choppers (which restores C19MC miRNA expression) restored the formation of NiC by >90%, and NiC influx of RPS6 and nucleolin to a similar extend upon Aurora-B inhibition (FIG. 3E-3G). Therefore, the influx and efflux of protein cargos are dependent on C19MC and might happen through nuclear pore complexes (NPCs) as nucleolin and ribosomal proteins (Li, Z. et al. Nature. 2023 618(7964):411-418).

### C19MC Drives Mitochondria Traffic to NiC

While an NPC-through traffic is known for protein-complexes, it cannot explain organelle influx such as mitochondria and potentially ribosomes (as RPS6 immunoreactivity could also include assembled ribosomes). Therefore, we hypothesized that, a microtubule organizing center (MTOC)-dependent push of cytoplasmic materials such as mitochondria into the nucle(ol)us could be the cause. Immunofluorescence for β-Tubulin demonstrated that, MTOC invades NiC in a small subset of ~5% cells but the remaining cells lacked β-Tubulin expression (FIG. 8C-8D). Mitochondria traffic into the NiC was suppressed >80% in SpCas9 control cells whereas it was restored by choppers under aurora-B inhibited conditions (FIG. 3H), demonstrating that, the NiC mitochondrial-cargo influx and efflux is also C19MC-dependent. Therefore, a genetic approach confirmed the C19MC-directed NiC-formation and cargo traffic.

### Autophagy Regulates NiC and MND

SpCas9 was differentially ubiquitinated and degraded by proteasomes between SpCas9 control and chopper cells (FIG. 3B-3C). The fate of ubiquitinated protein is alternatively decided by autophagy. Therefore, we examined if autophagy regulates NiC or not. Starvation-induced autophagy doubled the number of NiC positivity compared to fed controls under aurora-B inhibited conditions, while lagging half-way behind in DNA synthesis and nuclear size (FIG. 4A). Conversely under fed conditions, inhibition of autophagy also doubled the NiC-positivity (FIG. 4B). Overexpression of an autophagy-flux indicator construct pMRX-IP-GFP-LC3-RFP (Addgene plasmid: 84573) (Kaizuka, T. et al. Mol Cell. 2016 64(4):835-849) revealed that GFP-LC3 accumulates at perinuclear cytoplasm (FIG. 9A) in baseline levels, but upon Aurora-B inhibition autophagic degradation of GFP-LC3 happens in the cytoplasm but not inside NiC as GFP-LC3 accumulates within NiC (FIG. 4C). A deeper examination of nuclear pattern revealed that, autophagy is required for NCF ingression from the cytoplasmic side towards NiC, as inhibition of autophagy resulted in the accumulation of nuclei with NiC but without NCF, at the expense of cells with NCF/MND+NiC (FIG. 4D). The role of starvation-induced autophagy in limiting DNA-replication and associated doubling of NiC positive cells (FIG. 4a), and inhibition of NiC by CDK1 inhibitor (FIG. 2J) prompted us to examine the relationship of NiC to DNA-replication. Inhibiting CDK1 promoted DNA-synthesis to a level of triploid content while suppressing NiC generation (FIG. 4E). Consistently, in CRISPR-chopper cells, the cells with ploidy level DNA accumulation are prevented due to nuclear division (FIG. 4F). Therefore, autophagy plays a part in generating NiC and MND by helping the progression of NCF and stalling DNA synthesis.

### Nuclear Coverage of Mitochondria & CDK1

Next, we sought to examine the mechanism of mitochondria influx into the NiC. We hypothesize that, the mitochondria are attached to the nuclear membrane and are passively drawn into the NiC compartment with the nuclear membrane and that, this could passively bring ribosomes located within mitochondria (FIG. 9B). First, CRISPR-engineered cells revealed that, the total mitochondria content is regulated by C19MC, as SpCas9 suppressed mitochondrial load per cell compared to parental cells and was restored in chopper cells (FIG. 4G). We noted a perinuclear distribution of mitochondria in SpCas9 control cells but the parental and chopper cells exhibited a characteristic nuclear coverage of mitochondria (NCoM) (FIG. 4H). Autophagy or CDK1 inhibitions in SpCas9 cells restored NCoM and improved the mitochondrial load per cell (FIG. 4h-i). Consistently in MCF-7 cells, Aurora-B inhibition resulted in NCoM well-aligned with nuclear-membrane margination of DNA during MND (FIG. 9C) suggesting heterochromatinization (Ungricht, R. & Kutay, U. Nat Rev Mol Cell Biol. 2017 18(4):229-245). Therefore, nuclear tethering of mitochondria by C19MC determined the passive influx of mitochondria into the NiC compartment along with nuclear membrane in a CDK1 and autophagy-dependent manner (FIGS. 3H, 4H-41). A ring-type distribution of mitochondria at the luminal-periphery of NiC (FIG. 2E) supports this conclusion.

### Summary & Discussion

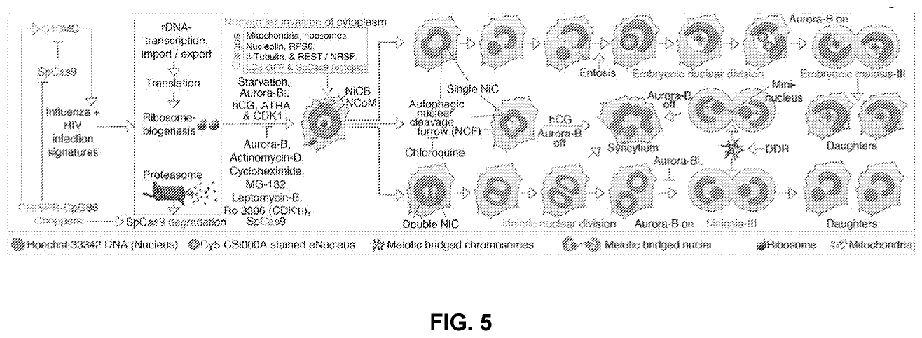

In summary, using TCGA pan-cancer differential gene expression profiling of C19MC^{High} tumors against C19MC^{Low} tumors, we discovered a biologically, chemically, and genetically validated mechanism for C19MC-driven meiotic nuclear division in C19MC^{High} cancer cells and tumors (FIG. 5). Nucle(ol)ar-invasion of mitochondria, and other cargoes results in autophagic ingression of NCF to proceed with MND (FIG. 5). MND is orchestrated by IAV+HIV infection-gene-signatures (rDNA, ribosome biogenesis pathway) by regulating the NiC cargo-traffic and generating autophagy immune zone (NiC). Generating autophagy immune zone gains more importance, as IAV uses its M2 protein to interact with LC3, to subvert autophagy to maintain virion stability (Beale, R. et al. Cell Host Microbe. 2014 15(2):239-47). Nucleolin localization at NiC suggests that, a plethora of viruses can use this mechanism for replication, as nucleolin is known to interact and internalize respiratory syncytial virus (RSV), enterovirus A 71 (EVA71), human parainfluenza virus type 3 (HPIV3), rabbit hemorrhagic disease virus (RHDV), Crimean-Congo hemorrhagic fever virus (CCHFV), coxsackievirus-B (CVB), HIV1, and IAV (Tonello, F., et al. Cell Mol Life Sci. 2022 79(5):271), or bind to the G-quadruplexes of Hepatitis-C virus (HCV) (Bian, W. X. et al. Nucleic Acids Res. 2019 47(1):56-68), HIV1 (Tosoni, E. et al. Nucleic Acids Res. 2015 43(18):8884-97), and Epstein-Barr virus (EBV) (Lista, M. J. et al. Nat Commun. 2017 8:16043).

MND is governed by Aurora-B and CDK1 to regulate the efflux and influx of protein and organelle cargoes to regulate NiC size, and NCF-driven division through autophagy. Organelle influx into NiC happens through the nucle(ol)ar-invasive cytoplasmic bridge (NiCB) by passive sliding of nuclear membrane tethered mitochondria (NiCB: FIGS. 2E, 5, 8F, 9B). Notably, we studied the C19MC function without using exogenous viruses, so it is clear that, NiC and MND are regulated by C19MC instead of viruses directly (viruses may yet do the same through C19MC or through rDNA/ribosome biogenesis pathway).

Suppression of C19MC by SpCas9 and its reversal by CRISPR-chopper that deletes CpG86 repeats reveals the pivotal role of CpG86 in C19MC regulation. This in part explains why pediatric cancers with high C19MC expression have the CpG86 relocated from C19MC by chromosomal rearrangements (Setty, B. A. et al. PLoS Genet. 2020 16(4):e1008642; Archer, T. C. & Pomeroy, S. L. Nat Genet. 2014 46(1):2-3).

Overall, these findings have far-reaching implications in cancer-pathogen interactions (viral restriction within tumor, bacterial suppression of antiviral response), restricting vertical transmission of viruses in human/primate placenta, CRISPR-therapeutics (SpCas9-based) and antiviral therapeutics. For example, the CDK1 inhibitor Ro3306 is shown to act on IAV M2-RNA splicing to restrict viral replication (Zhao, L. et al. Antiviral Res. 2022 201:105296), and inhibits the NiC in our study, a nucleolar-compartment implicated in viral replication (Hiscox, J. A. Arch Virol. 2002 147(6):1077-89).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for detecting nuclear invasion of cytoplasm (NiC) in a cell, comprising
a) contacting the cell with an effective amount of Aurora-B kinase inhibitor, and
b) staining the cell with a nuclear dye,
wherein detection of unstained regions within the nucleus of the cell is an indication of NiC, and
wherein detection of mitochondria, RPS6, nucleolin or REST is a confirmation of NiC.

2. The method of claim 1, wherein the cell is a parental or CRISPR-engineered cell.

3. The method of claim 1, wherein the Aurora-B kinase inhibitor comprises AZD-1152-HQPA, ZM 447439, PF-03814735, MK-0457, CCT137690, AMG 900, MLN8237, SNS-314 (mesylate), GSK1070916, Danusertib, CYC-116, CCT129202, or PHA-680632.

4. The method of claim 1, wherein the nuclear dye comprises Hoechst-33342.

5. The method of claim 1, further comprising contacting the cell with an effective amount of human chorionic gonadotropin (hCG), an agent that promotes steroidogenesis, or a combination thereof.

6. The method of claim 5, further comprising contacting the cell with an effective amount of all-trans-retinoic acid (ATRA).

7. The method of claim 5, further comprising contacting the cell with an Aurora-B inhibitor.

8. The method of claim 5, further comprising contacting the cell with an effective amount of arachidonic acid, diacylglycerol, or Phorbol 12-myristate 13-acetate (PMA), or a combination thereof.

9. The method of claim 1, further comprising contacting the cell with an effective amount of transcription inhibitor or translation inhibitor can inhibit NiC/NiCM and can be a stand-alone or combination cancer or antiviral therapeutic.

10. The method of claim 1, wherein the cell comprises a cancer cell.

11. The method of claim 1, further comprising contacting the cell with a mitochondrial dye or probe, wherein detection of mitochondria within the nuclear dye unstained regions within the nucleus is an indication of nuclear invasion of cytoplasmic mitochondria (NiCM) and is the validation of NiCM.

12-13. (canceled)

14. The method of claim 11, wherein the probe further comprises a detectable label.

15-16. (canceled)

17. The method of claim 14, wherein the detectable label comprises Cy5.

18. A method, comprising
a) contacting a cell with an effective amount of Aurora-B kinase inhibitor to induce NiC,
b) contacting the cell with a candidate agent, to inhibit NiC, and
b) staining the cell with a nuclear dye,
wherein reduction of unstained regions within the nucleus of the cell is an indication that Aurora-B kinase inhibitor-induced NiC is blocked by the candidate agent.

19. (canceled)

20. An RNA probe comprising the nucleic acid sequence CGUGCGGGCCCGGGCGUGC (SEQ ID NO:1), CGUGCCGCUACCGCUGCCA (SEQ ID NO:2), CGUGCUGACGCCACCAGAC (SEQ ID NO:3), CGUGCCCAUCAGCCCUGGG (SEQ ID NO:4), CCGUGCCCAUCACCGUGCC (SEQ ID NO:5), CGUGCAGCCGAGCGAGGCC (SEQ ID NO:6), GCGUGCGGCCAGUGAGGAC (SEQ ID NO:7), CGUGCAGGCCCCGCCCCCG (SEQ ID NO:8), CGUGCAGCCCCUGCUCUCA (SEQ ID NO:9), or CGUGCUUGGGGAGCUAGAG (SEQ ID NO:10) conjugated to a detectable label.

21-25. (canceled)

26. A method for detecting mitochondria within the nucleus of a cell, comprising transfecting the cell with an effective amount of the RNA probe of claim 18, contacting the cell with an effective amount of Aurora-Bi, and imaging the cell for the detectable label alone or in combination with a live cell DNA stain.

27. A method for delivering an RNA molecule of interest into the mitochondria of a cell, comprising transfecting or treating the cell with an effective amount of the RNA probe of claim 24, and contacting the cell with an effective amount of Aurora-Bi.

28. (canceled)

29. A set of CRISPR guide RNAs comprising the nucleic acid sequence
(SEQ ID NO: 11)
GGGCACGGTGGTTCCCATCAGGG
and
(SEQ ID NO: 12)
AGAGGGGTCCAGGCTGGGCACGG;
or
(SEQ ID NO: 13)
CTCAACAGAAGACCACCAAATGG
and
(SEQ ID NO: 14)
TCTCCCACCATTGTCTAGGCTGG.

30. A method for restoring anti-viral C19MC miRNA expression in a cell, comprising contact the cell with the set of guide RNA probes of claim 29 under conditions suitable for gene editing.
